(19) United States
(12) Patent Application Publication
(10) Pub. No.: US 2010/0239556 A1
(43) Pub. Date: Sep. 23, 2010

(54) PROMOTING ECM PRODUCTION BY FIBROBLAST CELLS AND/OR PROMOTING MIGRATION OF FIBROBLAST CELLS IN A BIOLOGICAL SYSTEM

(75) Inventors: Timothy Edward Rayner, Netherby South Australia (AU); Mark Orlando DeNichilo, Grange South Autralia (AU); Ronald Ian William Osmond, Henley Beach South South Autralia (AU); Aaron Thomas Mitchell, Prospect South Australia (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TGR BioScience Pty Ltd., Thebarton (AU)

(21) Appl. No.: 12/677,779

(22) PCT Filed: Sep. 12, 2007
(86) PCT No.: PCT/AU2007/001346

§ 371 Date: Jun. 7, 2010

Related U.S. Application Data
(60) Provisional application No. 60/869,848, filed on Dec. 13, 2006.
(60) Provisional application No. 60/871,739, filed on Dec. 22, 2006.
(60) Provisional application No. 60/883,119, filed on Jan. 2, 2007.

(51) Int. Cl. A61K 38/44; A61P 17/02; C12N 9/08

(52) U.S. Cl. 424/94.4; 435/192

(57) ABSTRACT

The present invention relates to a method of promoting production of one or more components of extracellular matrix by one or more fibroblast cells in a biological system and/or promoting migration of one or more fibroblast cells in a biological system. The method includes exposing one or more fibroblast cells in the biological system to an effective amount of an agent with peroxidase activity.

### FIELD OF THE INVENTION

The present invention relates to methods, compositions and substrates for promoting production of one or more components of extracellular matrix by fibroblast cells in a biological system, and to methods, compositions and substrates for promoting migration of fibroblast cells in a biological system.

### BACKGROUND OF THE INVENTION

The extracellular matrix (ECM) of vertebrates is a complex structural entity surrounding and supporting cells that are found in mammalian tissues. The ECM of mammalian tissues is composed of complex mixtures of structural proteins (collagen and elastin), specialized proteins (fibrillin, fibronectin and laminin) and, proteoglycans. The major fibrillar structural proteins, collagen and elastin, are responsible for tissue strength and resilience and play a dynamic role in promoting cell growth and differentiation. Collagen is the most abundant protein in the body and comprises about 50% of total body protein.

The macromolecules that constitute the extracellular matrix are produced, secreted and deposited by connective tissue cells, such as fibroblast cells which are widely distributed in the tissue.

ECM loss can occur under many conditions. For example, ECM loss leads to volume depletion and soft-tissue contour defects, deep wrinkles (rhytides), crows feet, nasolabial folds and marionette grooves. Volume depletion and soft-tissue contour defects can result from atrophic conditions, post-acne scars, traumatic scars, surgical scars, chicken pox scars.

Generally, filler substances are used in the treatment of such conditions. Filler substances can be autologous (that is, derived from the subject themselves), heterologous (of animal or human origin) or alloplastic (biomaterials of a chemical nature). Filler substances are generally injected or implanted into the area of skin requiring augmentation. They can be used for cosmetic enhancement such as lip augmentation, rhinoplasty, malar and submalar augmentation, chin augmentation, tear-trough contouring, liposuction defects, orbital cavity augmentation, oral soft-tissue ridge augmentation, nipple augmentation and phalloplasty.

However, there remain a number of deficiencies in the treatment of ECM loss. For example, obtaining quantities of ECM protein such as collagen for commercial applications can involve extracting the protein from sources such as skin, hoof or the like of a bovine or porcine origin. These materials are generally undesirable for use in humans due to their risk of causing disease and immunogenic reactions. Human tissue on the other hand also carries some risk of disease transmission and is available only in limited quantities. In vitro means of producing non-immunogenic human collagen in cell culture are generally not considered to be commercially viable due to the high expense of cell culture and the low yield of collagen.

There are also a number of situations where it would be beneficial to provide augmentation, repair and/or healing of tissues. For example, wounds present a situation where under many circumstances augmentation and/or repair of the wound would be beneficial. Examples of wounds that would benefit from agents that aid in augmentation, repair and/or healing include partial and full-thickness wounds, pressure ulcers, venous ulcers, diabetic ulcers, chronic vascular ulcers, surgical wounds (donor sites/grafts, post-Moh's surgery, post-laser surgery, podiatric, dehisced wounds), trauma wounds (abrasions, lacerations, second and third-degree burns, skin tears) and draining wounds.

The recruitment of a variety of specialised cell types to the site of a wound is a critical part of the process of wound healing. This process requires extracellular matrix and basement membrane deposition, angiogenesis, selective protease activity and re-epithelialisation. One critical component of the healing process is the stimulation of fibroblast cells to generate the extracellular matrix. This extracellular matrix constitutes a major component of the connective tissue that develops to repair the wound area. There is a continuing need to develop methods and medicaments that promote the healing of wounds.

As such, there is a need for new treatments for conditions involving ECM loss, or for conditions that would benefit from tissue augmentation and/or repair. The present invention relates to methods and compositions for treating such conditions.

A reference herein to a patent document or other matter which is given as prior art is not to be taken as an admission that the document or matter was known or that the information it contains was part of the common general knowledge as at the priority date of any of the claims.

### SUMMARY OF THE INVENTION

The present invention is based on the finding that agents with peroxidase activity are able to stimulate the production of various components of extracellular matrix by fibroblast cells in vitro and in vivo. In addition, agents with peroxidase activity are able to promote the migration of fibroblast cells, and thus may be used to populate a region with fibroblast cells.

Thus, the present invention may be used to promote production of one or more components of extracellular matrix in a tissue in need of treatment and/or to promote migration of one or more fibroblast cells into the tissue in need of treatment. As such, agents with peroxidase activity may be used to promote a fibrogenic response in vivo, and therefore may be used to promote one or more of tissue generation, tissue regeneration, tissue repair and tissue support.

In this regard, the present studies also demonstrate that not only can the agent be delivered to fibroblast cells by way of directly introducing the agent into a tissue, but a fibrogenic response can also be promoted by delivering the agent in conjunction with a substrate. In this case, deposition of extracellular matrix and/or migration of fibroblast cells into the substrate is promoted by the action of the agent with peroxidase activity.

Accordingly, the present invention provides a method of promoting production of one or more components of extracellular matrix by one or more fibroblast cells in a biological system and/or promoting migration of one or more fibroblast cells in a biological system, the method including exposing the one or more fibroblast cells in the biological system to an effective amount of an agent with peroxidase activity.

The present invention also provides a method of promoting a fibrogenic response in a tissue in a subject, the method including delivering to one or more fibroblast cells in the tissue an effective amount of an agent with peroxidase activity.

The present invention also provides a method of promoting one or more of tissue generation, tissue regeneration, tissue repair and tissue support in a tissue in a subject, the method including delivering to the tissue an effective amount of an agent with peroxidase activity.

The present invention also provides a method of treating a subject in need of one or more of tissue generation, tissue regeneration, tissue repair and tissue support, the method including delivering to a tissue in the subject in need of treatment an effective amount of an agent with peroxidase activity.

The present invention also provides a method of promoting infiltration of a three dimensional substrate including one or more interstices with one or more fibroblast cells, the method including associating the three dimensional substrate with an agent with peroxidase activity and exposing the substrate with the agent with peroxidase activity to one or more fibroblast cells, thereby promoting infiltration of the interstices of the substrate with the one or more fibroblast cells.

The present invention also provides a substrate for one or more of tissue generation, tissue regeneration, tissue repair and tissue support, the substrate including an agent with peroxidase activity coupled to, or associated with, the substrate.

The present invention also provides a method of promoting deposition of one or more components of extracellular matrix in a three dimensional substrate including one or more interstices, the method including associating the three dimensional substrate with an agent with peroxidase activity and exposing the substrate with the agent with peroxidase activity to one or more fibroblast cells, thereby promoting deposition of one or more components of extracellular matrix in the interstices of the substrate by the one or more fibroblast cells.

The present invention also provides use of an agent with peroxidase activity in the preparation of a medicament for treating a subject in need of one or more of tissue generation, tissue regeneration, tissue repair and tissue support.

The present invention also provides a composition for treating a subject in need of one or more of tissue generation, tissue regeneration, tissue repair and tissue support, the composition including an agent with peroxidase activity.

The present invention also provides a method of identifying an agent that promotes production of one or more components of extracellular matrix by a fibroblast cell in a biological system and/or promotes migration of a fibroblast cell in a biological system, the method including:
(i) providing an agent with peroxidase activity;
(ii) determining the ability of the agent to promote the production of one or more components of extracellular matrix by a fibroblast cell in a biological system and/or the ability of the agent to promote migration of a fibroblast cell in a biological system; and
(iii) identifying an agent that promotes production of one or more components of extracellular matrix by a fibroblast cell and/or promotes migration of a fibroblast cell in a biological system.

The present invention also provides a method of promoting production of one or more components of extracellular matrix by one or more fibroblast cells in a biological system and/or promoting migration of one or more fibroblast cells in a biological system, the method including exposing the one or more fibroblast cells in the biological system to an effective amount of a polypeptide with an amino acid sequence as provided by the polypeptides defined by an EC number selected from the following group consisting of EC 1.11.1.1; EC 1.11.1.2; EC 1.11.1.3; 1.13.11.11; EC 1.11.1.5; EC 1.11.1.7; EC 1.11.1.8; EC 1.11.1.9; EC 1.11.1.10; EC 1.11.1.12; EC 1.11.1.13; EC 1.11.1.14; EC 1.11.1.15; EC 1.11.1.16; or an active fragment or variant of any of the aforementioned.

Various terms that will be used throughout the specification have meanings that will be well understood by a skilled addressee. However, for ease of reference, some of these terms will now be defined.

The term “a tissue in need of one or more of tissue generation, tissue regeneration, tissue repair and tissue support” as used throughout the specification is to be understood to mean a tissue that would benefit from one or more of (i) deposition of one or more components of extracellular matrix in the tissue, and/or in a region near the tissue; (ii) infiltration of fibroblast cells into the tissue, and/or into a region near the tissue; (iii) a fibrogenic response in the tissue, and/or in a region near the tissue; (iv) and a tissue that would generally benefit from the introduction of a substrate and/or cells to augment the tissue.

The term “substrate” as used throughout the specification is to be understood to mean a a three dimensional material, and includes for example a liquid, a gel, a semi-solid material, or a solid material.

The term “subject” as used throughout the specification is to be understood to mean a human or animal subject. It will be understood that the present invention includes within its scope veterinary applications. For example, the animal subject may be a mammal, a primate, a livestock animal (eg. a horse, a cow, a sheep, a pig, or a goat), a companion animal (eg. a dog, a cat), a laboratory test animal (eg. a mouse, a rat, a guinea pig, a bird), an animal of veterinary significance, or an animal of economic significance.

In this regard, a subject in need of treatment includes a subject suffering from, or susceptible to, a condition for example arising from or leading to ECM loss or absence, such as a subject having soft-tissue volume depletion and/or soft-tissue contour defects, birth/developmental soft tissue defects, genetic soft tissue defects, deep wrinkles (rhytides), crows feet, nasolabial folds and marionette grooves. Volume depletion and soft-tissue contour defects can result from atrophic conditions, post-acne scars, traumatic scars, surgical scars, chicken pox scars. In addition, a subject in need of treatment includes a subject suffering from, or susceptible to, wounds such as partial and full-thickness wounds, pressure ulcers, venous ulcers, diabetic ulcers, chronic vascular ulcers, surgical wounds (donor sites/grafts, post-Moh's surgery, post-laser surgery, podiatric, dehisced wounds), trauma wounds (abrasions, lacerations, second and third-degree burns, skin tears) and draining wounds.

It will be appreciated that the present invention therefore can be used, for example, for wound healing, cosmetic enhancement such as lip augmentation, rhinoplasty, malar and submalar augmentation, chin augmentation, tear-trough contouring, liposuction defects, orbital cavity augmentation, oral soft-tissue ridge augmentation, nipple augmentation and phalloplasty.

The term “promote” as used throughout the specification is to be understood to mean an increase in the progress of a process, including any one or more of the start, rate, probability, continuation or termination of a process.

The term “fibrogenic response” as used throughout the specification is to be understood to mean either or both of the accumulation of fibroblast cells and the production ECM by the fibroblast cells within a localised, treated area of tissue. The accumulation of fibroblast cells can occur via one or more of increased migration of fibroblast cells into the localised, treated area of tissue, the proliferation of fibroblast cells and/or the differentiation of fibroblast cells, or cells such as myofibroblast cells and fibrocytes into ECM producing cells.

The term “biological system” as used throughout the specification is to be understood to mean a multi-cellular system. For example, the biological system may be an isolated tissue or organ, a tissue or organ in a subject, or an entire human or animal subject, such as a human or animal subject in need of treatment for, or susceptible to, one or more of tissue generation, tissue regeneration, tissue repair and tissue support.

The term “migration of a fibroblast cell” as used throughout the specification is to be understood to mean that the effect of an agent with peroxidase activity on a fibroblast cell is to promote movement of the fibroblast cells cells from one location to another location. Without being bound by theory, it appears that one function of an agent with peroxidase activity is to directly and/or indirectly facilitate chemotaxis of the fibroblast cell. For example, under some conditions the agent acts as a chemotactic agent for facilitating movement of the fibroblast cell from a region of lower peroxidase concentration/activity to a region with higher peroxidase concentration/activity.

### BRIEF DESCRIPTION OF THE FIGURES

FIG. 1 shows the effect of a range of proteins having peroxidase activity on soluble collagen I, III and VI production by confluent human foreskin fibroblast (HFF) cells after 72 hours. Data points show a dose representing the most effective response achieved from a range of doses tested for each protein having peroxidase activity, and are the combined results from 3-6 experiments. The data has been converted to show a fold-change from unstimulated control cells for each experiment and statistically significant differences compared to the control cells are shown by the asterix.

FIG. 2 shows the effect of a range of proteins having peroxidase activity on soluble collagen I production by confluent human foreskin fibroblast (HFF) cells after 5 and 7 days. Data points show a dose representing the most effective response achieved from a range of doses tested for each protein having peroxidase activity, and are the combined results from 3-5 experiments. Statistically significant differences compared to untreated control cells are shown by the asterix.

FIG. 3 shows the effect of myeloperoxidase (MPO) and ascorbate peroxidase (APX) on soluble collagen I production and cell proliferation by confluent human foreskin fibroblast (HFF) cells after 72 hours. Data points show a dose representing the most effective response achieved from a range of doses tested for each protein having peroxidase activity, and are the combined results from 3-4 experiments. The data has been converted to show a fold-change from unstimulated control cells for each experiment and statistically significant differences compared to the control cells are shown by the asterix.

FIG. 4 shows a silver stained protein gel demonstrating the effect of proteins having peroxidase activity (SBP, MP and LP compared to unstimulated control cells) on the amount of total proteinaceous material incorporated into the extracellular matrix after 72 hours by confluent adult (donor) fibroblast cells.

FIG. 5 shows the effect of restimulating confluent human foreskin fibroblast (HFF) cells treated with 10% FBS, TGF beta 2 (TGF) and proteins with peroxidase activity (MP, ARP, LP). Media was collected after two days and the cells retreated or left untreated for a further 5 days. The data has been converted to show a fold-change from unstimulated control cells and data points show the change in collagen I in the media two days after the first stimulation and after an additional five days (with or without a second stimulation), and are the mean of three determinations±SEM.

FIG. 6 shows the effect on the production of collagen I by confluent human foreskin fibroblast (HFF) cells of a double stimulation with 10% FBS, TGF beta 2 (TGF) and proteins with peroxidase activity (MP, ARP, LP). The total amount of collagen I produced by cells stimulated twice (day 0-2 and day 2-7) was calculated and compared with cells stimulated only once (day 0-7). Data points are the mean of three determinations±SEM.

FIG. 7 shows the degree of cell infiltration into a section of INTEGRA® Dermal Regeneration Template incubated with human adult (donor) fibroblast cells for 7 days under control culture conditions. The section was stained with an antibody against smooth-muscle actin (SMA) and the arrows show SMA-positive fibroblast cells.

FIG. 8 shows the degree of cell infiltration into a section of INTEGRA® incubated with human adult (donor) fibroblast cells for 14 days under control culture conditions. The section was stained with an antibody against smooth-muscle actin (SMA) and the arrows show SMA-positive fibroblast cells.

FIG. 9 shows the effect on the degree of cell infiltration into INTEGRA® of treating the INTEGRA with SBP prior to and during the incubation of the INTEGRA with human adult (donor) fibroblast cells for 7 days. The section was stained with an antibody against smooth-muscle actin (SMA) and the arrows show SMA-positive fibroblast cells.

FIG. 10 shows the effect on the degree of cell infiltration into INTEGRA® of treating the INTEGRA with HRP prior to and during the incubation of the INTEGRA with human adult (donor) fibroblast cells for 14 days. The section was stained with an antibody against smooth-muscle actin (SMA) and the arrows show SMA-positive fibroblast cells.

FIG. 11 shows the effect on the degree of cell infiltration into INTEGRA® of treating the INTEGRA with SBP prior to only (pre-treat only), prior to and during (pre+post treat), and during only (post-treat only), the incubation of the INTEGRA with human foreskin fibroblast (HFF) cells for 6 days. Sections of INTEGRA from each condition and an untreated control were stained with an antibody against smooth-muscle actin (SMA) and the SMA-positive fibroblast cells counted in at least three fields of view. The combined mean±SEM of the cell counts for each condition and the control are shown.

FIG. 12 shows the degree of ECM production in a section of INTEGRA® incubated with human adult (donor) fibroblast cells for 7 days under control culture conditions. The section was stained with an antibody against collagen I and the arrows show areas of cell-associated collagen I staining, as opposed to the ribbon-like staining of the structural scaffold of the INTEGRA.

FIG. 13 shows the degree of ECM production in a section of INTEGRA® incubated with human adult (donor) fibroblast cells for 14 days under control culture conditions. The section was stained with an antibody against collagen I and the arrows show areas of cell-associated collagen I staining, as opposed to the ribbon-like staining of the structural scaffold of the INTEGRA.

FIG. 14 shows the effect on the degree of ECM production in a section of INTEGRA® of treating the INTEGRA with SBP prior to and during the incubation of the INTEGRA with human adult (donor) fibroblast cells for 7 days. The section was stained with an antibody against collagen I and the arrows show areas of cell-associated collagen I staining, as opposed to the ribbon-like staining of the structural scaffold of the INTEGRA.

FIG. 15 shows the effect on the degree of ECM production in a section of INTEGRA® of treating the INTEGRA with HRP prior to and during the incubation of the INTEGRA with human adult (donor) fibroblast cells for 14 days. The section was stained with an antibody against collagen I and the arrows show areas of cell-associated collagen I staining, as opposed to the ribbon-like staining of the structural scaffold of the INTEGRA.

FIG. 16 shows the effect on the production of soluble collagen I by human foreskin fibroblast (HFF) cells populating a piece of INTEGRA® and treated with SBP for 24 hours. The soluble collagen I released into the media surrounding the INTEGRA was measured by ELISA 72 hours after stimulation with SBP. Also shown is the amount of collagen I released into the media of an identical piece of INTEGRA containing HFF cells that was not treated with SBP (control).

FIG. 17 shows the effect of SBP on the incorporation of collagen I into the ECM deposited by human foreskin fibroblast (HFF) cells encapsulated within a three-dimensional hydrogel matrix. The cells were incubated with or without SBP and supplemented with different levels of FBS for 7 days, after which the cell-associated matrix was extracted and collagen I content determined by Western blot.

FIG. 18 shows the organisation of collagen fibers formed by human adult (donor) cells when treated with proteins having peroxidase activity. Cells were grown on microscope coverslips, treated with proteins having peroxidase activity for seven days (HRP shown) and the coverslips stained using an antibody against collagen I. The arrows show the long, parallel collagen fibers formed by the cells as part of the cell-associated ECM.

FIG. 19 shows the ability of proteins having peroxidase activity to be eluted from a collagen matrix. Pieces of INTEGRA® were pre-treated with or without SBP for 30 minutes or 16 hours, washed, and then transferred to the media bathing human foreskin fibroblast (HFF) cells. The amount of SBP released into the media after 24 and 48 hours (elution period) was measured using a peroxidase activity assay and the amount of collagen I production (by the HFF cells) stimulated by the eluted SBP measured by ELISA. The results show the responses obtained from pieces of INTEGRA incubated with SBP compared to pieces of INTEGRA incubated without SBP (Cont).

FIG. 20 shows the effect of horseradish peroxidase conjugated to sheep-anti-rabbit (SAR) and donkey-anti-sheep (DAS) antibodies on soluble collagen I induction and growth of HFF cells after 72 hours. Data points are means of three determinations±SEM.

FIG. 21 shows a representative image of a cross-section of the dermis and subcutaneous adipose layer from rat skin collected 3 days after a control injection of bovine serum albumin (50 μg in 50 μl). The tissue section was stained with haematoxylin and eosin and the image collected at ×20 magnification.

FIG. 22 shows the tissue response after 3 days to a bolus dose of lactoperoxidase (LP; 125 μg in 50 μl) injected into rat skin. The skin section was stained with haematoxylin and eosin and the image collected at ×20 magnification. The arrow shows an area representative of the tissue response.

FIG. 23 shows the tissue response after 3 days to a bolus dose of transforming growth factor β2 (TGFβ2; 0.5 μg in 50 μl) injected into rat skin. The tissue section was stained with haematoxylin and eosin and the image collected at ×20 magnification. The arrow shows an area representative of the tissue response.

FIG. 24 shows the dose effect of lactoperoxidase (5-125 μg) injected into the skin of rats compared to a control injection as determined by the tissue fibroblast reaction after 3 days. Haematoxylin and eosin stained sections were scored in a blinded fashion by an experienced pathologist for the presence of fibroblast cells (1+-4+). Data represents the mean±SEM of scores from 8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 25 shows the dose effect of lactoperoxidase (5-125 μg) injected into the skin of rats compared to a control injection as determined by the deposition of collagen-rich extracellular matrix (ECM) after 3 days. Masson's trichrome and haematoxylin and eosin stained sections were scored in a blinded fashion by an experienced pathologist for the presence of collagen-rich ECM associated with the tissue fibroblast reaction (1+-3+). Data represents the mean±SEM of scores from 8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 26 shows the dose effect of horseradish peroxidase (1.25-25 μg) injected into the skin of rats compared to a control injection as determined by the tissue fibroblast reaction after 3 days. Haematoxylin and eosin stained sections were scored in a blinded fashion by an experienced pathologist for the presence of fibroblast cells (1+-4+). Data represents the mean±SEM of scores from 4-8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 27 shows the dose effect of horseradish peroxidase (1.25-25 μg) injected into the skin of rats compared to a control injection as determined by the deposition of collagen-rich extracellular matrix (ECM) after 3 days. Masson's trichrome and haematoxylin and eosin stained sections were scored in a blinded fashion by an experienced pathologist for the presence of collagen-rich ECM associated with the tissue fibroblast reaction (1+-3+). Data represents the mean±SEM of scores from 4-8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 28 shows the dose effect of micro-peroxidase (5-125 μg) injected into the skin of rats compared to a control injection as determined by the tissue fibroblast reaction after 3 days. Haematoxylin and eosin stained sections were scored in a blinded fashion by an experienced pathologist for the presence of fibroblast cells (1+-4+). Data represents the mean±SEM of scores from 8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 29 shows the dose effect of micro-peroxidase (5-125 μg) injected into the skin of rats compared to a control injection as determined by the deposition of collagen-rich extracellular matrix (ECM) after 3 days. Masson's trichrome and haematoxylin and eosin stained sections were scored in a blinded fashion by an experienced pathologist for the presence of collagen-rich ECM associated with the tissue fibroblast reaction (1+-3+). Data represents the mean±SEM of scores from 4-8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 30 shows the dose effect of soybean peroxidase (1.25-25 μg) injected into the skin of rats compared to a control injection as determined by the tissue fibroblast reaction after 3 days. Haematoxylin and eosin stained sections were scored in a blinded fashion by an experienced pathologist for the presence of fibroblast cells (1+-4+). Data represents the mean±SEM of scores from 4-8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 31 shows the dose effect of soybean peroxidase (1.25-25 μg) injected into the skin of rats compared to a control injection as determined by the deposition of collagen-rich extracellular matrix (ECM) after 3 days. Masson's trichrome and haematoxylin and eosin stained sections were scored in a blinded fashion by an experienced pathologist for the presence of collagen-rich ECM associated with the tissue fibroblast reaction (1+-3+). Data represents the mean±SEM of scores from 4-8 rats. Statistical analysis was performed using a Kruskal-Wallis one way analysis of variance on ranks (ANOVA) and post-hoc t-test comparison with the control group using Dunnetts method. Significant differences are shown (*p<0.05).

FIG. 32 shows the degree of cellular integration and ECM production expected one month after implantation of Restylane into the dermis of a rat. A tissue section was stained with an antibody against collagen I and areas of fibroblast-associated collagen I positive staining are shown by the arrows.

FIG. 33 shows the effect of combining lactoperoxidase (LP; 1 mg/ml) with Restylane on the degree of cellular integration and ECM production one month after implantation of the filler substance into the dermis of a rat. A tissue section was stained with an antibody against collagen I and areas of fibroblast-associated collagen I positive staining are shown by the arrows.

FIG. 34 shows the effect of combining horseradish peroxidase (HRP; 500 μg/ml) with Restylane on the degree of cellular integration and ECM production one month after implantation of the filler substance into the dermis of a rat. A tissue section was stained with an antibody against collagen I and areas of fibroblast-associated collagen I positive staining are shown by the arrows.

FIG. 35 shows the degree of cellular integration and ECM production expected one month after implantation of Hylaform into the dermis of a rat. A tissue section was stained with an antibody against collagen I and areas of fibroblast-associated collagen I positive staining are shown by the arrows.

FIG. 36 shows the effect of combining lactoperoxidase (LP; 1 mg/ml) with Hylaform on the degree of cellular integration and ECM production one month after implantation of the filler substance into the dermis of a rat. A tissue section was stained with an antibody against collagen I and areas of fibroblast-associated collagen I positive staining are shown by the arrows.

FIG. 37 shows the effect of combining horseradish peroxidase (HRP; 500 μg/ml) with Hylaform on the degree of cellular integration and ECM production one month after implantation of the filler substance into the dermis of a rat. A tissue section was stained with an antibody against collagen I and areas of fibroblast-associated collagen I positive staining are shown by the arrows.

FIG. 38 shows the effect of combining horseradish peroxidase (HRP; 25 μg) with hyaluronic acid (HA) based dermal filler substances, or lactoperoxidase (LP; 50 μg) with Sculptra (poly-L-lactic acid), as determined by the tissue fibroblast reaction after 7 days. Haematoxylin and eosin stained sections were scored in a blinded fashion by an experienced pathologist for the presence of fibroblasts (1+-4+). Data represents the mean±SEM of scores from 4-8 rats.

FIG. 39 shows the effect of combining horseradish peroxidase (HRP; 25 μg) with hyaluronic acid (HA) based dermal filler substances, or lactoperoxidase (LP; 50 μg) with Sculptra (poly-L-lactic acid), as determined by the deposition of collagen-rich extracellular matrix (ECM) after 7 days. Masson's trichrome and haematoxylin and eosin stained sections were scored in a blinded fashion by an experienced pathologist for the presence of collagen-rich ECM associated with the tissue fibroblast reaction (1+-3+). Data represents the mean±SEM of scores from 4-8 rats.

FIG. 40 shows the effect of a range of proteins with peroxidase activity on collagen I levels in human skin. Collagen I immunohistochemistry was performed on tissue cross-sections from human skin injected with the proteins with peroxidase activity or a vehicle control and collected after 3 days. The relative staining intensity was scored in a blinded fashion (1+-5+).

FIG. 41 shows the effect of a range of proteins with peroxidase activity on collagen III levels in human skin. Collagen III immunohistochemistry was performed on tissue cross-sections from human skin injected with the proteins with peroxidase activity or a vehicle control and collected after 3 days. The relative staining intensity was scored in a blinded fashion (1+-5+).

FIG. 42 shows after 72 hours the effect of treating HFF cells with dexamethasone (Dex; 20 μM) on peroxidase from Arthromyces ramosus (ARP) and transforming growth factor beta 2 (TGFβ2) induced soluble collagen I levels. Data points are means of three determinations±SEM.

### GENERAL DESCRIPTION OF THE INVENTION

As discussed above, in one embodiment the present invention provides a method of promoting production of one or more components of extracellular matrix by one or more fibroblast cells in a biological system and/or promoting migration of one or more fibroblast cells in a biological system, the method including exposing the one or more fibroblast cells in the biological system to an effective amount of an agent with peroxidase activity.

The present invention is based on the finding that agents with peroxidase activity are able to stimulate the production of various components of extracellular matrix by fibroblast cells in vitro and in vivo. In addition, agents with peroxidase activity are able to promote the migration of fibroblast cells, and thus such agents may be used to promote population of a biological tissue or a substrate with fibroblast cells.

For example, the present invention may be used to promote production of one or more components of extracellular matrix in a tissue in need of treatment and/or to promote migration of one or more fibroblast cells into a tissue in need of treatment.

As such, agents with peroxidase activity may also be used to promote a fibrogenic response in vivo, and therefore may be used to promote one or more of tissue generation, tissue regeneration, tissue repair and tissue support. For example, the method may be used to promote one or more of regeneration, repair and/or healing of a wound, to rectify a dermal deficit, and to augment a tissue.

In addition, the present invention may be used to promote tissue integration generally, and to promote healing and/or repair.

The agent with peroxidase activity in the various embodiments of the present invention may be exposed to the one or more fibroblast cells in a biological system by a suitable method. For example, the fibroblast cells may be exposed to the agent by delivering the agent directly to the fibroblast cells, such as by way of directly contacting the cells with the agent. Such methods include topical administration of the agent to cells, or introduction of the agent into a tissue directly, for example by injection or implantation. Alternatively, the agent may be delivered to the fibroblast cells, for example, by indirect administration, such as systemic administration. Other methods of delivery are discussed in detail below.

Alternatively, the agent may be delivered to the fibroblast cells in conjunction with a substrate. In the case where the substrate allows migration of fibroblast cells into the substrate, this can be used to promote deposition of one or more components of extracellular matrix in the substrate and/or to promote migration of fibroblast cells into the substrate. Such substrates may be used for treating a subject with a tissue in need of one or more of generation, regeneration, repair, or support.

As described above, in one embodiment the present invention may be used to promote the production of one or more components of extracellular matrix.

In this regard, the term “production” (or variants thereof) as used throughout the specification is to be understood to mean the formation of a particular product at one or more specific locations. An increase in production can be achieved, for example, by one or more of an increase in the synthesis, expression, excretion, secretion and deposition of a product in a particular location.

The one or more components of extracellular matrix in the various embodiments of the present invention include one or more collagen I, collagen III and collagen VI and other collagen types, elastin, fibronectin, laminins, tenascin, perlecan, proteoglycan, hyaluronic acid, glycosaminoglycans, a non-collagenous extracellular matrix protein, de novo extracellular matrix, or a functional component thereof.

In one embodiment, the one or more components of extracellular matrix are selected from one or more of the group consisting of collagen I, collagen III, collagen IV, collagen VI, fibronectin, elastin, laminin, proteoglycan, hyaluronic acid, and de novo extracellular matrix, or a functional component thereof.

In this regard, a functional component of extracellular matrix is to be understood to mean one or more components of extracellular matrix that act in a similar fashion to extracellular matrix in a particular setting.

Thus, in one embodiment, the present invention may be used, for example, to promote one or more of generation, regeneration, repair and/or healing of a wound, to rectify a dermal deficit, and to augment a tissue.

As described above, the present invention may also be used to promote migration of a fibroblast cell. For example, the present invention may be used to promote migration of fibroblast cells into a substrate in vitro or in vivo, thus allowing population of the substrate with fibroblast cells. In the case of substrates with populated fibroblast cells produced in vitro, such substrates may be used for introduction into a subject to treat a tissue in need of one or more of tissue generation, tissue regeneration, tissue repair and tissue support.

Accordingly, in another embodiment the present invention provides a substrate for one or more of tissue generation, tissue regeneration, tissue repair and tissue support, the substrate including an agent with peroxidase activity coupled to, or associated with, the substrate.

In this regard, the term “associated” is understood to mean that at least a proportion of the agent with peroxidase activity has some working inter-relationship with the substrate, such as being located in/on the substrate, or releasably or non-releasably attached to the substrate.

In one embodiment, the substrate is permeable. Thus, at least some of the agent with peroxidase activity may be able to diffuse into, and/or out of, the substrate.

In one specific embodiment, the substrate is treated with an agent with peroxidase activity before use of the substrate in a subject.

In the case where the agent with peroxidase activity is coupled to the substrate, the coupling may be direct or indirect, and involve covalent and/or non-covalent means of coupling. Methods for coupling agents to substrates are known in the art.

For example a collagen specific antibody can be used to attach a peroxidase conjugated antibody to extracellular matrix (or to a collagen-based tissue regeneration scaffold). As demonstrated in the present studies, proteins having peroxidase activity coupled to an antibody stimulate extracellular matrix production when applied to fibroblast cells cells.

In one embodiment, the substrate is a liquid, a gel, a semi-solid substrate, or a solid substrate.

In one embodiment, the substrate is biocompatible and/or biodegradable. For example, the substrate may be suitable for use in a subject for one or more of tissue generation, tissue regeneration, tissue repair and tissue support. Details of such substrates are discussed further below.

In one specific embodiment, the substrate is selected from one or more of the group consisting of extracellular matrix, an ECM derived three dimensional matrix, a tissue substitute, a natural or synthetic biological replacement tissue, an allograft, an autograft, a wound closure device, a xenograft, a skin substitute, a natural or synthetic three dimensional polymer, and a wound dressing.

In another embodiment, the substrate includes a filler substance, or is a filler substance. Examples of filler substances include one or more of a collagen, hyaluronic acid, poly-L-lactic acid, fat including autologous fat, calcium hydroxyapatite, a natural or synthetic polymer, donor tissue including autologous donor tissue or heterologous donor tissue, and extracellular matrix or a component thereof.

The substrate in the various embodiment of the present invention may further include one or more other agents.

For example, the substrate may include one or more of a steroidal anti-inflammatory drug, a calcineurin inhibitor, an anti-histamine, an antibiotic, an anti-microbial agent, a growth factor, a growth promoting agent, an angiogenic promoter, a protease inhibitor, an anti-oxidant, an anaesthetic agent, an analgesic agent, and a chemotactic agent.

The substrate in the various embodiments of the present invention may also include one or more of fibroblast cells, keratinocytes, endothelial cells and cells capable of producing adnexia. Such cells may be autologous or heterologous.

The substrate may be treated with an agent with peroxidase activity. In one embodiment, the substrate is treated with an agent with peroxidase activity at one or more of (i) before inclusion of fibroblast cells in the substrate; (ii) commensurate with inclusion of fibroblast cells in the substrate; and (iii) after inclusion of fibroblast cells in the substrate.

As previously discussed herein, in one embodiment the substrate is pre-treated with the agent with peroxidase activity.

In this regard, it will be also appreciated that fibroblast cells to be included with the substrate may also be exposed to an agent with peroxidase activity at one or more of (i) before inclusion in the substrate; (ii) commensurate with inclusion in the substrate; and (iii) after inclusion in the substrate.

The present invention also provides a method of treating a subject in need of one or more of tissue generation, tissue regeneration, tissue repair and tissue support, the method including delivering to the subject an effective amount of an agent with peroxidase activity and/or a substrate as described herein.

As discussed previously herein, the present invention may also be used to promote deposition of one or more components of extracellular matrix in a tissue or in a substrate.

In one embodiment, the present invention may be used to promote deposition of one or more components of extracellular matrix in the interstices of a three dimensional substrate, in vitro or in vivo.

Accordingly, in another embodiment the present invention provides a method of promoting deposition of one or more components of extracellular matrix in a three dimensional substrate including one or more interstices, the method including associating the three dimensional substrate with an agent with peroxidase activity and exposing the substrate with the agent with peroxidase activity to one or more fibroblast cells, thereby promoting deposition of one or more components of extracellular matrix in the interstices of the substrate by the one or more fibroblast cells.

As discussed previously herein, the present invention may also be used to promote migration of fibroblast cells into a tissue or into a suitable substrate. Thus, in one embodiment the present invention may be used, for example, to promote deposition of fibroblast cells in a tissue and thereby promote repair, healing and augmentation of the tissue in a subject.

In another embodiment, the present invention may be used to promote migration of fibroblast cells into a substrate, for example a substrate present in a tissue. Thus, the present invention may also be used, for example, to populate a substrate in vivo with fibroblast cells and thereby promote repair, healing and augmentation of the tissue in a subject.

As discussed previously herein, the present invention is based on exposure of fibroblast cells in a biological system to an agent with peroxidase activity.

In one embodiment, the biological system is a human or animal subject.

As previously described herein, agents with peroxidase activity may also be used to promote a fibrogenic response in vivo, and therefore may be used to promote one or more of tissue generation, tissue regeneration, tissue repair and tissue support.

Accordingly, in another embodiment the present invention provides a method of promoting a fibrogenic response in a tissue in a subject, the method including delivering to one or more fibroblast cells in the tissue an effective amount of an agent with peroxidase activity.

In another embodiment, the present invention provides a method of promoting one or more of tissue generation, tissue regeneration, tissue repair and tissue support in a tissue in a subject, the method including delivering to the tissue an effective amount of an agent with peroxidase activity.

In a further embodiment, the present invention provides a method of treating a subject in need of one or more of tissue generation, tissue regeneration, tissue repair and tissue support, the method including delivering to a tissue in the subject in need of treatment an effective amount of an agent with peroxidase activity.

An agent with peroxidase activity may also be used in the preparation of a medicament for treating a subject in need of one or more of tissue generation, tissue regeneration, tissue repair and tissue support.

Accordingly, in another embodiment the present invention provides use of an agent with peroxidase activity in the preparation of a medicament for treating a subject in need of one or more of tissue generation, tissue regeneration, tissue repair and tissue support.

Methods for the preparation of medicaments are known in the art, and are discussed in further detail below.

The one or more fibroblast cells in the various embodiments of the invention are one or more cells that are capable of producing one or more components of extracellular matrix. Examples of such cells include fibroblast cells, myofibroblast cells and fibrocytes. In this regard, a fibroblast cell in the various embodiments of the present invention includes within its scope a cell that is a progenitor of a fibroblast cell

Depending upon the circumstances, the one or more fibroblast cells may further be endogenous cells present in the biological system, and/or exogenous cells introduced into the biological system.

The one or more fibroblast cells may also be autologous cells or heterologous cells.

The one or more fibroblast cells may also be engineered cells, such as cells engineered to express and/or secrete a protein with peroxidase activity. Methods for engineering cells to express and/or secrete proteins are known in the art. For example, see Sambrook et al. Molecular Cloning: A Laboratory Manual (2d ed., Cold Spring Harbor Laboratory Press, Cold Spring Harbor, N.Y. (1989) and Ausubel, F. M. et al. (1989) Current Protocols in Molecular Biology, John Wiley & Sons, New York, N.Y.

For example, a mammalian cell can be transformed in vitro with an expression construct encoding a protein having a peroxidase activity, suitable to stimulate the at least one fibroblast cell to produce a component of extracellular matrix.

Methods to obtain the constitutive or transient expression of gene products engineered into stromal cells are described for example in U.S. Pat. No. 5,962,325. Suitable promoters and transcriptional control regions are also described therein.

The term “fibrogenic response” as used throughout the specification means either or both of the accumulation of fibroblast cells and the production ECM by the fibroblast cells within a localised, treated area of tissue. The accumulation of fibroblast cells can occur via one or more of increased migration of fibroblast cells into the localised, treated area of tissue, the proliferation of fibroblast cells and/or the differentiation of fibroblast cells, or cells such as myofibroblast cells and fibrocytes into ECM producing cells.

As discussed previously herein, the present invention may also be used to promote infiltration of a tissue or a substrate with fibroblast cells.

For example, in one embodiment the method may be used to promote infiltration of a suitable substrate, such as a gel, a semi-solid substrate or a three dimensional substrate including one or more interstices with fibroblast cells.

In one embodiment, the present invention provides a method of promoting infiltration of a three dimensional substrate including one or more interstices with one or more fibroblast cells, the method including associating the three dimensional substrate with an agent with peroxidase activity and exposing the substrate with the agent with peroxidase activity to one or more fibroblast cells, thereby promoting infiltration of the interstices of the substrate with the one or more fibroblast cells.

In one embodiment, the one or more fibroblast cells are in vitro. Thus the method may be used to populate a three dimensional substrate in vitro with fibroblast cells, and/or to deposit one or more components of extracellular matrix produced from fibroblast cells in the interstices of a three dimensional substrate.

In another embodiment, the one or more fibroblast cells are in a biological system, for example, in a human or animal subject. Thus the method may be used, for example, to populate a substrate in vivo with fibroblast cells.

The present invention may also be used to promote deposition of one or more components of extracellular matrix in a tissue or substrate, either in vitro or in vivo.

In one embodiment, the substrate is a three dimensional substrate with one or more interstices and the present invention used to promote deposition of extracellular matrix, or a component thereof, in the interstices of the substrate.

The term “agent with peroxidase activity” in the various embodiments of the present invention is an agent having an activity which catalyzes a reaction of the form:

ROOR′+electron donor(2e−)+2H+→ROH+R′OH.

Examples of agents with peroxidase activity include polypeptide peroxidases (eg peroxidase enzymes, or functional fragments and/or variants thereof) and non-polypeptide peroxidases. Non-polypeptide examples of agents with peroxidase activity include; manganese(III) 5,10,15,20-tetraphenyl porphyrin in aqueous poly(sodium styrene-4-sulfonate-co-2-vinylnaphthalene) polymer, manganese dioxide, DNA-hemin complex (PS2.M-hemin), RNA-hemin complex (rPS2.M-hemin), supramolecular-hydrogel-encapsulated hemin, hemin chemically bonded to N,NA-methylenebisacrylamide-cross-linked-Nisopropylacrylamide-(poly(NIPAAm)/MBA/hemin), non-immobilised metalloderivatives of anionic tetrasulphonated tetraphenylporphyrin (MTPPS, M=manganese(III), iron(III), cobalt(III)), and metalloderivatives of anionic tetrasulphonated tetraphenylporphyrin (MTPPS, M=manganese(III), iron(III), cobalt(III)) immobilized on cationically functionalized divinylbenzene (DVB)-crosslinked polystyrene (PS).

In one embodiment, the agent with peroxidase activity is a protein with peroxidase activity. Protein peroxidases may contain a heme cofactor in their active sites, or redox-active cysteine or selenocysteine residues.

It will be appreciated that a protein with peroxidase activity includes a polypeptide with peroxidase activity, including an enzyme, a fragment of a peroxidase enzyme or protein, and a natural or synthetic variant of a protein with peroxidase activity.

In this regard, the term “variant” as used throughout the specification is to be understood to mean an amino acid sequence of a polypeptide or protein that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids. A variant may also be a form of the protein that has one or more deleted amino acids (eg a truncated form of the protein), and/or a form of the protein that has one or more additional exogenous amino acids (eg a form of the protein fused to another polypeptide sequence). It will be appreciated that a variant will therefore include within its scope a fragment of a protein.

Generally, the variant will be a functional variant, that is, a variant that retains the functional ability of the progenitor protein.

Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Under some circumstances, substitutions within the aliphatic group alanine, valine, leucine and isoleucine are also considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative.

A peroxidase enzyme is classified as an oxidoreductase and has an EC number (Enzyme commission number) of EC 1.11.1. Peroxidases are widely distributed in nature and have been isolated from animals including, for example, humans (eg lactoperoxidase, glutathione peroxidase, myeloperoxidase, thyroid peroxidase, microperoxidase), plants (eg horseradish peroxidase and soybean peroxidase), yeast (eg cytochrome c peroxidase), fungi (eg Arthromyces ramosus peroxidase) and bacteria (eg catalase peroxidases).

Accordingly, in another embodiment the present invention provides a method of promoting production of one or more components of extracellular matrix by one or more fibroblast cells in a biological system and/or promoting migration of one or more fibroblast cells in a biological system, the method including exposing the one or more fibroblast cells in the biological system to an effective amount of a polypeptide with an amino acid sequence as provided by the polypeptides defined by an EC number selected from the following group consisting of EC 1.11.1.1; EC 1.11.1.2; EC 1.11.1.3; 1.13.11.11; EC 1.11.1.5; EC 1.11.1.7; EC 1.11.1.8; EC 1.11.1.9; EC 1.11.1.10; EC 1.11.1.12; EC 1.11.1.13; EC 1.11.1.14; EC 1.11.1.15; EC 1.11.1.16; or an active fragment or variant of any of the aforementioned.

Details of polypeptides defined by the above EC numbers are as described in Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively).

The amino acid sequences of the relevant polypeptides can be readily obtained by a person skilled in the art.

Further, other proteins which are not classified as a peroxidase enzyme may have peroxidase activity, such as for example cytochrome P450 (CYP). CYP also has the ability to catalyse oxidation/reduction reactions and is considered to be a protein with peroxidase activity. The peroxidase activity of CYP proteins can be retained when protein fragments containing the heme group are generated such as those prepared from cytochrome c from equine heart. In addition the peroxidase activity of cytochrome C₅₅₂ from Marinobacter hydrocarbonoclasticus can be retained when protein fragments containing the heme group are generated using proteinase K. These “microperoxidases” consist of the heme structure with between 5 and 11 amino acids attached, and due to their smaller size are likely to be more permeable and therefore accessible to biological tissues.

In this regard, it will be appreciated that the peroxidases in the various embodiments of the present invention also include polypeptides that utilise a metal substitution of the heme group, or which are heme-independent.

In one specific embodiment, the protein with peroxidase activity is selected from the group consisting of lactoperoxidase, horseradish peroxidase, soybean peroxidase, myeloperoxidase, ascorbate peroxidase, micro-peroxidase and Arthromyces ramosus peroxidase.

Methods are known in the art for producing agents with peroxidase activity.

It will be appreciated that the agent with peroxidase activity can be in the form of a substantially pure agent, or as part of a mixture with one or more components. For example, a protein with peroxidase activity can be provided as a biological fluid including one or more proteins with the activity.

Proteins with peroxidase activity can be isolated and extracted from animals including humans (eg lactoperoxidase; LPO, eosinophil peroxidase; EPO, glutathione peroxidase, myeloperoxidase; MPO, thyroid peroxidase, microperoxidase), plants (eg horseradish peroxidase and soybean peroxidase), yeast (eg cytochrome c peroxidase), fungi (eg Arthromyces ramosus peroxidase) and bacteria (eg catalase peroxidases). These extracts can be crude or enriched for the protein with peroxidase activity. MPO, EPO and LPO are unique in that they are primarily found in granules (lysosomes) of neutrophils, eosinophils and secretory cells of exocrine glands respectively. These proteins with peroxidase activity can be obtained from biological fluids of animal origin. MPO and EPO are released into the phagocytic vacuole of the neutrophils or eosinophils as well as into the blood plasma, and LPO is secreted into milk, saliva and tears.

Proteins with peroxidase activity can also be recombinantly expressed. For example, lactoperoxidase can be expressed in CHO cells. Recombinant human lactoperoxidase and thyroid peroxidase can also be expressed by recombinant baclovirus (Autographs californica nuclear polyhedrosis virus; AcNPV) infection of Spodoptera frugiperda (Sf9) or Tricoplusia ni (High5) insect cells. Recombinant cytosolic ascorbate peroxidase can be expressed in peas, horseradish peroxidase and soybean peroxidase can be expressed in E. coli (or other expression systems) and Arthromyces ramosus peroxidase can be produced via recombinant heterologous expression in systems such as Aspergillis.

Methods are known in the art for recombinant expression of proteins. See for example Sambrook et al. Molecular Cloning: A Laboratory Manual (2d ed., Cold Spring Harbor Laboratory Press, Cold Spring Harbor, N.Y. (1989) and Ausubel, F. M. et al. (1989) Current Protocols in Molecular Biology, John Wiley & Sons, New York, N.Y.

Autologous proteins with peroxidase activity can be obtained from a subject by a method known in the art. The protein can be concentrated if desired. Proteins with peroxidase activity are obtainable, for example, from red blood cells (glutathione peroxidase, peroxiredoxin), platelets (glutathione peroxidase), white blood cells (eosinophil peroxidase, myeloperoxidase, peroxiredoxin) and milk (lactoperoxidase).

Agents with peroxidase activity can also be purchased commercially from general chemical suppliers such as Sigma-Aldrich (Castle Hill, NSW, Australia) or from specialised companies such as Biozymes Pty Ltd (Wales, UK).

Methods for identifying agents with peroxidase activity, and for determining their activity, are known in the art. For example, the peroxidase activity of a protein may be measured using an enzyme substrate assay such as the sigmafast™ OPD detection system.

The one or more fibroblast cells in the various embodiments of the present invention may also be exposed to one or more other agents, such as a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent.

Accordingly, the agent with peroxidase activity may be exposed to the one or more fibroblast cells in conjunction with one or more of these aforementioned agents.

In the case of one or more fibroblast cells in a tissue in a subject, in one embodiment the exposure of the one or more fibroblast cells to the agent with peroxidase activity is by delivery of the agent to a tissue in need of treatment in the human or animal subject.

In one embodiment, the tissue is a soft tissue. In this regard, it will be appreciated that the present invention may be used to augment a soft tissue.

In this regard, a soft tissue includes for example a non-bony, non-cartilaginous, non-tendinous fibrous connective tissues (such as the dermis of the skin), subcutaneous adipose and muscular tissues, and body organs and their associated structures, but excluding epithelial tissues such as those forming the skin (keratinised stratified epithelium), lining the alimentary canal and forming the secretions of internal organs. A soft tissue also refers to endogenous or exogenous fibroblast-derived tissue that may be generated or deposited to fill tissue spaces, voids or cavities

The amount of the agent with peroxidase activity that the one or more fibroblast cells are exposed to in the biological system in the various embodiments of the present invention is not particularly limited, and generally will be in the range such that the cells will be exposed to a concentration from about 0.1 μg/ml to 1 mg/ml.

In one embodiment, the one or more fibroblast cells are exposed to an agent with peroxidase activity in the range from 1 to 600 μg/ml. Further suitable ranges include 1 to 500 μg/ml, 1 to 200 μg/ml and 1 to 125 μg/ml.

In one embodiment, the one or more fibroblast cells are exposed to a microperoxidase in an amount of about 5-600 μg/ml, and typically in the following ranges: 10-20 μg/ml, 50-80 μg/ml, 100-150 μg/ml, 220-280 μg/ml and 450-550 μg/ml.

In another embodiment, the one or more fibroblast cells are incubated with a soybean peroxidase in an amount of about 0.2-200 μg/ml, and typically in the following ranges: 0.2-30 μg/ml and 40-150 μg/ml.

In another embodiment, the one or more fibroblast cells are incubated with a horseradish peroxidase in an amount of about 0.2-200 μg/ml, and typically in the following ranges: 0.2-30 μg/ml, and 40-150 μg/ml.

In another embodiment, the one or more fibroblast cells are incubated with an Arthromyces ramosus peroxidase in an amount of about 0.2-200 μg/ml, typically in the following ranges: 0.2-30 μg/ml and 40-150 μg/ml.

In another embodiment, the one or more fibroblast cells are incubated with lactoperoxidase in an amount of about 5-400 μg/ml, typically in the following ranges: about 5-100 μg/ml, 130-180 μg/ml and 280-330 μg/ml.

In another embodiment, the one or more fibroblast cells are incubated with a myeloperoxidase in an amount of about 10-40 μg/ml, typically about 30 μg/ml.

In another embodiment, the one or more fibroblast cells are incubated with an ascorbate peroxidase in an amount of about 1-800 μg/ml, typically in the following ranges: about 2-100 μg/ml, 120-200 μg/ml and 250-800 μg/ml.

The agent with peroxidase activity may be delivered in a form and at a concentration suitable to allow the agent to reach the desired site of action and have the desired effect.

In this regard, the effective amount of the agent with peroxidase activity to be administered to the biological system (eg a subject) is not particularly limited, so long as it is within such an amount and in such a form that generally exhibits a useful, beneficial or therapeutic effect. The amount to be administered will depend on the particular characteristics of the treatment, the mode of administration, and the characteristics of the subject, such as general health, other diseases, age, sex, genotype, and body weight. A person skilled in the art will be able to determine appropriate dosages depending on these and other factors.

The exposure may be within any time suitable to produce the desired effect. In a human or animal subject, the agent with peroxidase activity may be delivered, for example, by direct implantation (eg by injection or as part of a substrate), orally, parenterally, topically or by any other suitable means, and therefore transit time of the agent must be taken into account.

As discussed previously herein, the agent with peroxidase activity may be delivered in conjunction with one or more other agents, such as a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent.

In one embodiment, the delivery of the agent is by implantation into the tissue in need of treatment and/or by implantation in a region near the tissue in need of treatment. In this regard, a region near a tissue in need of treatment will be understood to mean a region, that when the agent with peroxidase activity is delivered to, provides a beneficial effect on the tissue in need of treatment.

Examples of implantation in the various embodiments of the present invention include injection, and/or introduction of the peroxidase in conjunction with a substrate.

In another embodiment, delivery of the agent with peroxidase activity is by topical administration to the tissue in need of treatment and/or by topical administration to a region near the tissue in need of treatment. Topical compositions are discussed in further detail below.

In one embodiment, the agent with peroxidase activity is delivered in conjunction with a substrate. Examples of substrates include a liquid, a gel, a semi-solid substrate, or a solid substrate.

As discussed previously, in one embodiment the agent with peroxidase activity is associated with a substrate. As previously described here, the In this regard, the term “associated” means that at least a proportion of the agent with peroxidase activity has some working inter-relationship with the substrate, such as being located in/on the substrate, or releasably or non-releasably attached to the substrate.

For example, the agent with peroxidase activity may be part of the substrate, located within one or more interstices in the substrate, and directly or indirectly contacted, or attached, to the substrate.

In one embodiment, the agent with peroxidase activity is releasable from the substrate upon delivery of the substrate to a subject. For example, the agent with peroxidase activity may elute from substrate when the substrate is in contact with a tissue.

In one embodiment, the substrate is biocompatible and/or biodegradable.

In one embodiment, the substrate is a template for one or more of tissue generation, tissue regeneration, tissue repair, tissue support and a filler substance. For example, the template may be extracellular matrix, an ECM derived three dimensional matrix, a tissue substitute, a natural or synthetic biological replacement tissue, an allograft, an autograft, a wound closure device, a xenograft, a skin substitute, a natural or synthetic three dimensional polymer, and a wound dressing.

In the case where the substrate is a filler substance, the filler substance may be a dermal filler (often also referred to as “dermal augmentation material”). Such materials include heterologous filler substances, alloplastic filler substances and autologous filler substances. General properties that filler substances may possess include materials which are slowly resorbable, elastic, inert, reactive, or non-resorbable and typically possess acceptable biocompatibility with the tissue in the area. Filler substances that are useful include a variety of human, bovine, porcine and ovine collagens, hyaluronic acid, poly-L-lactic acid, autologous fat, calcium hydroxyapatite, synthetic and biocompatible polymers, donor cells and donor tissue.

In one embodiment, the filler substance includes one or more of a collagen, hyaluronic acid, poly-L-lactic acid, fat including autologous fat, calcium hydroxyapatite, a natural or synthetic polymer, donor tissue including autologous donor tissue or heterologous donor tissue, and extracellular matrix or a component thereof.

In one specific embodiment, the template is a three dimensional substrate including one or more interstices. For example, the template may be extracellular matrix, an ECM derived three dimensional matrix, a tissue substitute, a natural or synthetic biological replacement tissue, an allograft, an autograft, a wound closure device, a xenograft, a skin substitute, a natural or synthetic three dimensional polymer, and a wound dressing.

As discussed previously herein, in one embodiment the substrate is pre-treated with an agent with peroxidase activity at one or more of prior to delivery to the tissue in need of treatment, commensurate with delivery to the tissue in need of treatment, and after delivery to the tissue in need of treatment.

In one embodiment, the agent with peroxidase activity is also delivered with one or more exogenous fibroblast cells, keratinocytes, endothelial cells, and cells capable of producing adnexia.

In one embodiment, the one or more exogenous fibroblast cells, keratinocytes, endothelial cells, and cells capable of producing adnexia are autologous to the biological system.

In one embodiment, the one or more exogenous fibroblast cells are pre-treated with an agent with peroxidase activity prior to delivery to the subject.

As previously discussed herein, the agent with peroxidase activity may be delivered in a form and at a concentration suitable to allow the agent to reach the desired site of action and have the desired effect.

In one embodiment the agent with peroxidase activity may be delivered in the form of a composition.

Accordingly, in another embodiment the present invention provides a composition for treating a subject in need of one or more of tissue generation, tissue regeneration, tissue repair and tissue support, the composition including an agent with peroxidase activity.

In one embodiment, the composition further includes one or more additional agents, for example one or more of a steroidal anti-inflammatory drug, a calcineurin inhibitor, an anti-histamine, an antibiotic, am anti-microbial agent, a growth factor, a growth promoting agent, an angiogenic promoter, a protease inhibitor, an anti-oxidant, an anaesthetic agent, an analgesic agent, and a chemotactic agent.

Accordingly, in another embodiment the present invention provides a composition suitable for delivery to a subject, the composition including an agent with peroxidase activity and one or more of a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent.

The composition may also include one or more cells. Examples of cells include one or more of fibroblast cells, keratinocytes, endothelial cells and cells capable of producing adnexia. In one embodiment, the cells are autologous.

In another embodiment, the agent with peroxidase activity in the composition is associated with a substrate.

In one embodiment, the agent is releasable from the substrate upon delivery of the substrate to a subject.

Examples of suitable are as previously described herein. In one embodiment, the substrate in the composition is a liquid, a gel, a semi-solid substrate, or a solid substrate.

In one specific embodiment, the substrate in the composition is a three dimensional substrate including one or more interstices, such as a substrate selected from the group consisting of extracellular matrix, an ECM derived three dimensional matrix, a tissue substitute, a natural or synthetic biological replacement tissue, an allograft, an autograft, a wound closure device, a xenograft, a skin substitute, a natural or synthetic three dimensional polymer, and a wound dressing.

In another embodiment, the composition further includes a filler substance, such as one or more of a collagen, hyaluronic acid, poly-L-lactic acid, fat including autologous fat, calcium hydroxyapatite, a natural or synthetic polymer, donor tissue including autologous donor tissue or heterologous donor tissue, and extracellular matrix or a component thereof.

The substrate in the composition may also includes one or more of fibroblast cells, keratinocytes, endothelial cells and cells capable of producing adnexia. In one embodiment, the one or more fibroblast cells, keratinocytes and cells capable of producing adnexia are autologous.

In the case of the composition including one or more fibroblast cells, and/or in the case of a substrate including one or more fibroblast cells, the cells may have been exposed to an agent with peroxidase activity.

In one embodiment, the one or more fibroblast cells are exposed to the agent with peroxidase activity at one or more of prior to delivery to a subject, commensurate with delivery to a subject, and after delivery to a subject.

In the case of a substrate including fibroblast cells, the cells may be treated with an agent at one or more of before inclusion in the substrate, commensurate with inclusion in the substrate, and after inclusion in the substrate.

In one embodiment, the composition is suitable for introduction into a subject, such as being suitable for implantation or injection into a subject.

In another embodiment, the composition is a topical composition.

In either case, the composition may, for example, form either part of a wound closure device, all or part of a dressing, or part of a dressing.

The present invention also provides a method of treating a subject in need of one or more of tissue generation, tissue regeneration, tissue repair and tissue support, by delivering to the subject an effective amount of a composition as previously described herein.

Methods for the preparation of pharmaceutical compositions are known in the art, for example as described in Remington's Pharmaceutical Sciences, 18th ed., 1990, Mack Publishing Co., Easton, Pa.; U.S. Pharmacopeia: National Formulary, 1984, Mack Publishing Company, Easton, Pa.; and M. E. Aulton, Pharmaceutics, The Science of Dosage Form Design, 2nd ed., Churchill Livingstone, Edinburgh, 2002.

Therapeutic delivery of biomolecules is generally as described in Bladon, C. (2002) “Pharmaceutical Chemistry: Therapeutic Aspects of Biomolecules” John Wiley & Sons Ltd.

As discussed previously herein, delivery of the agent with peroxidase activity may be, for example, by direct injection into or near the desired site of action, by implantation into or near the desired site of action, by intravenous delivery, by intraperitoneal delivery, by intradermal delivery by subcutaneous delivery, by intramuscular delivery, orally, or topically.

As described above, the delivery of a composition including an agent with peroxidase activity may also include the use of one or more pharmaceutically acceptable additives, including pharmaceutically acceptable salts, amino acids, polypeptides, polymers, solvents, buffers, excipients, preservatives and bulking agents, taking into consideration the particular physical, microbiological and chemical characteristics of the agent to be administered.

For example, the agent with peroxidase activity can be prepared into a variety of pharmaceutically acceptable compositions in the form of, e.g., an aqueous solution, an oily preparation, a fatty emulsion, an emulsion, a gel, a lyophilised powder for reconstitution, etc. and can be administered as a sterile and pyrogen free injection into a tissue, or as an embedded preparation or as a transmucosal preparation through nasal cavity, rectum, uterus, vagina, lung, etc. The composition may be administered in the form of oral preparations (for example solid preparations such as tablets, caplets, capsules, granules or powders; liquid preparations such as syrup, emulsions, dispersions or suspensions).

Compositions containing the agent with peroxidase activity may also contain one or more pharmaceutically acceptable preservative, buffering agent, diluent, stabiliser, chelating agent, viscosity-enhancing agent, dispersing agent, pH controller, solubility-modifying agent or isotonic agent. These excipients are known to those skilled in the art.

Examples of suitable preservatives are benzoic acid esters of para-hydroxybenzoic acid, phenols, phenylethyl alcohol or benzyl alcohol. Examples of suitable buffers are sodium phosphate salts, citric acid, tartaric acid and the like. Examples of suitable stabilisers are antioxidants such as alpha-tocopherol acetate, alpha-thioglycerin, sodium metabisulphite, ascorbic acid, acetylcysteine, 8-hydroxyquinoline, and chelating agents such as disodium edetate. Examples of suitable viscosity enhancing agents, suspending, solubilizing or dispersing agents are substituted cellulose ethers, substituted cellulose esters, polyvinyl alcohol, polyvinylpyrrolidone, polyethylene glycols, carbomer, polyoxypropylene glycols, sorbitan monooleate, sorbitan sesquioleate, polyoxyethylene hydrogenated castor oil 60.

Examples of suitable pH controllers include hydrochloric acid, sodium hydroxide, buffers and the like. Examples of suitable isotonic agents are glucose, D-sorbitol or D-mannitol, sodium chloride.

The administration of the agent with peroxidase activity may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, glidant, anti-adherent, binder, flavorant or sweetener, taking into account the physical, chemical and microbiological properties of the agent being administered.

For these purposes, the composition may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes intradermal, subcutaneous, intravenous, intramuscular, intraperitoneal, intrathecal, intraventricular, intrasternal, and intracranial injection or infusion techniques.

When administered parenterally, the composition will normally be in a unit dosage, sterile, pyrogen free injectable form (solution, suspension or emulsion, which may have been reconstituted prior to use) which is usually isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing or wetting agents, complexing agents, polymers, solubility aids and suspending agents.

The sterile injectable forms may also be sterile injectable solutions or suspensions in non-toxic parenterally acceptable diluents or solvents, for example, as solutions in 1,3-butanediol. Among the pharmaceutically acceptable vehicles and solvents that may be employed are water, ethanol, glycerol, saline, dimethylsuphoxide, N-methylpyrrolidone, dimethylacetamide, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain additives, such as substances that enhance solubility, isotonicity, and chemical stability, for example anti-oxidants, buffers and preservatives.

In addition, the composition containing the agent may be in a form to be reconstituted prior to administration. Examples include lyophilization, spray drying and the like to produce a suitable solid form for reconstitution with a pharmaceutically acceptable solvent prior to administration.

Compositions may include one or more buffer, bulking agent, isotonic agent and cryoprotectant and lyoprotectant. Examples of excipients include, phosphate salts, citric acid, non-reducing sugars such as sucrose or trehalose, polyhydroxy alcohols, amino acids, methylamines, and lyotropic salts are preferred to the reducing sugars such as maltose or lactose.

When administered orally, the agent will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include excipients such as lactose, dextrose, sucrose, sorbitol, mannitol, starches, gum acacia, calcium phosphate, mineral oil, cocoa butter, oil of theobroma, alginates, tragacanth, gelatin, syrup, substituted cellulose ethers, polyoxyethylene sorbitan monolaurate, methyl hydroxybenzoate, propyl hydroxybenzoate, talc, magnesium stearate, and the like.

A tablet may be made by compressing or moulding the agent optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the agent may also utilize controlled release technology.

For topical administration, the composition and/or substrates may be in the form of a solution, spray, lotion, cream (for example a non-ionic cream), gel, paste or ointment. Alternatively, the composition may be delivered via a liposome, nanosome, ribosome, or nutri-diffuser vehicle.

As discussed previously herein, the administration of the agent may further include the administration of one or more other agents, such as a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent.

The administration of such agents may occur at the same time and in the same manner as the administration of the agent with peroxidase activity. Alternatively, the administration of the agent with peroxide activity may be separate to the administration of such additional agents, and occur at an appropriate time before or after administration of the agent.

Thus, the present invention also provides a combination product for separate or co-administration of an agent with peroxidase activity and one or more agents selected from the group consisting of a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent.

Accordingly, in another embodiment the present invention provides a combination product including the following components: (i) an agent with peroxidase activity; and (ii) an agent selected from the group consisting of a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent and a chemotactic agent, wherein the components provided in a form for co-administration to a subject or in a form for separate administration to a subject.

The components of the combination product may be packaged separately or together in suitably sterilized containers such as ampoules, bottles, or vials, either in multi-dose or in unit dosage forms. The containers are typically hermetically sealed. Methods are known in the art for the packaging of the components.

As discussed previously herein, co-administration can be sequential or simultaneous and generally means that the agents are present in the subject during a specified time interval. Typically, if a second agent is administered within the half-life of the first agent, the two agents are considered co-administered.

As previously described herein, an agent with peroxidase activity may also be administered with one or more components of extracellular matrix, for example an ECM protein such as collagens and elastin, which are suitable for administering to an animal as soft tissue filler substances (see for example U.S. Pat. No. 5,705,488). Examples of injectable formulations of ECM proteins such as collagens and elastin are commercially available and are described for use as soft tissue filler substances, primarily heterologous or alloplastic filler substances.

In relation to compositions including a protein with peroxidase activity, such compositions will typically include one or more of a pharmaceutically acceptable diluent, carrier or excipient. Acceptable carriers or diluents for therapeutic use are well known in the pharmaceutical art, and are described for example in Remington's Pharmaceutical Sciences Mack Publishing Co. (A. R. Gennaro edit. 1985). The choice of pharmaceutical carrier excipient or diluent can be selected with regard to the intended route of administration and standard pharmaceutical practice. The compositions may include as, or in addition to, the carrier, excipient or diluent, any suitable binder, lubricant, suspending agent (liposomes), coating agent, or solubilizing agent.

It is known in the art that there may be different composition/formulation requirements dependent on the different delivery systems. For example, agents with peroxidase activity may be dissolved in saline. Alternatively these proteins can be made up in a solution provided with a two part liquid-powder that is mixed before use.

Implantable (subcutaneous) slow release capsules (such as used for contraceptives like Implanon™ or Depo-Provera™ injections) are also applicable for administration of a composition of the present invention.

In another example, the composition of the present invention may be formulated to be delivered using an implanted mini-pump wherein the composition is typically administered by continuous infusion into the desired location.

In an alternative embodiment, compositions of the invention can be injected or otherwise implanted parenterally for example, subcutaneously or intradermally. In one specific embodiment the formulation is administered subcutaneously or intradermally.

Subcutaneous and intradermal formulations can also contain one or more additional agents such as for example filler substances, lidocaine (local anaesthetic/analgesic), matrix metalloproteinase inhibitors, antioxidants and anti-inflammatory agents (corticosteroids).

Formulations for intradermal delivery of a proteinaceous material typically include one or more of the following ingredients: albumin, buffer, buffered saline, buffered salt solution, and anaesthetic/analgesic (typically local).

Examples of intradermal formulations suitable for injection into an animal or human are described below:

(i) Agents with peroxidase activity dissolved in sterile water.

(ii) Agents with peroxidase activity dissolved in 0.9% sterile saline (150 mM NaCl); +/− human albumin (0.01%-0.5%); +/− local anaesthetic/analgesic (e.g. bupivacaine hydrochloride (1.25-5 mg/ml); +/− adrenaline acid tartrate (0.0045-0.0091 mg/ml); +/− lidocaine (0.5-2%); +/− epinephrine (1:100,000-1:200,000); +/− fibroblast cells.

(iii) Agents with peroxidase activity dissolved in phosphate buffered saline (137 mM NaCl, 2.7 mM KCl, 10 mM phosphate buffer; 150 mM NaCl, 150 mM NaH2PO4/Na2HPO4) +/− human albumin (0.01-0.5%); +/− local anaesthetic/analgesic; +/− fibroblast cells.

(iv) Agents with peroxidase activity dissolved in phosphate-citrate buffer (50 mM) +/− sodium perborate (0.03%); +/− human albumin (0.01-0.5%); +/− local anaesthetic/analgesic; +/− fibroblast cells.

(v) Agents with peroxidase activity dissolved in a solution of sterile water containing carboxymethylcellulose (2.7%) and mannitol.

(vi) Agents with peroxidase activity incorporated into biocompatible polyalkymide hydrogels (eg Bio-Alcamid® made by Polymekon S.r.l. (Milan, Italy).

(vii) Agents with peroxidase activity incorporated into an animal derived hyaluronic acid gel (eg Hylaform® Plus).

(viii) Agents with peroxidase activity incorporated into stabilised (and non-stabilised) non-animal derived hyaluronic acid gel (e.g. Restylane®, Perlane, Macrolane etc).

(ix) Agents with peroxidase activity incorporated into powders of poly-L-lactic acid microspheres, carboxymethylcellulose and mannitol (e.g. Sculptra®).

(x) Agents with peroxidase activity incorporated into sterile water for dissolving powders of poly-L-lactic acid microspheres, carboxymethylcellulose and mannitol (e.g. Sculptra®).

(xi) Agents with peroxidase activity incorporated into blends of absorbable medical polymers such carboxymethylcellulose and polyethylene oxide (eg Laresse™)

(xii) Agents with peroxidase activity incorporated into bovine (eg Zyderm® and Zyplast®), ovine, porcine (eg Permacol), avian or marine collagens.

(xiii) Agents with peroxidase activity incorporated into cell-culture derived human collagen (eg Cosmoderm®, Cosmoplast®).

(xiv) Agents with peroxidase activity incorporated into recombinantly derived human collagen (eg rh collagen I and III).

(xv) Agents with peroxidase activity incorporated into homogenous polymethylmethacrylate microspheres evenly suspended in a solution of partly denatured bovine collagen and lidocaine (eg ArteFill).

(xvi) Agents with peroxidase activity incorporated into a cross-linked polyacrylamide gel (eg Aquamid).

(xvii) Agents with peroxidase activity incorporated into highly purified injectable long-chain polydemethylsiloxane silicone oil (eg Silikon 1000).

(xviii) Agents with peroxidase activity incorporated into a suspension of calcium hydroxylapatite microspheres in a gel containing sodium carboxymethylcellulose and glycerin (eg Radiesse).

(xix) Agents with peroxidase activity incorporated into a suspension of autologous or non-autologous tissue (eg adipose tissue).

(xx) Agents with peroxidase activity incorporated into a suspension of non-collagenous ECM proteins (eg elastin).

(xxi) Agents with peroxidase activity incorporated dissolved in a solution of Krebs-Ringer's solution containing NaCl 118.1 mM; KCl 3.4 mM; CaCl₂ 2.5 mM; MgSO₄ 0.8 mM; KH₂PO₄ 1.2 m; NaHCO₃ 25.0 mM; Glucose 11.1 mM; +/− fibroblast cells.

Compositions of the invention may include a filler substance in addition to (i) agents with peroxidase activity; (ii) in addition to fibroblast cells previously treated with agents with peroxidase activity; or (iii) untreated fibroblast cells in addition to agents with peroxidase activity. Heterologous filler substances include for example bovine collagen. Formulations currently available on the world market comprise for example 35 mg bovine collagen/ml (Zyderm I™), or 65 mg/ml bovine collagen/ml (Zyderm II™) Other examples of heterologous filler substances include, for example, a form of bovine collagen slightly cross-linked by a low concentration of gluteraldehyde. Preparations of hyaluronic acid (a constituent of ground substance) and its derivatives, synthetic forms or chemical modified variants are also used as filler substances, as are suspensions of human collagen fibrils, elastin fibers and glycosaminoglycans derived from cadaver tissue. Another example includes an acellular, structurally and biochemically intact human dermal graft also derived from human allograft skin that is freeze-dried without damaging the extracellular matrix and acts as template for recipient cell repopulation. Preserved fascia grafts (pFGs) harvested predominantly from fascia lata of human cadavers is prepared as an injectable, particulated suspension and once implanted, provokes host fibroblasts to replace the graft over several months with a vascularised sheet of native collagen. Each of the heterologous filler substances are usually only viewed as temporary treatments as volume persistence or graft longevity are restricted to between 6 and 24 months at the very longest with breakdown and resorption of the implanted foreign material during graft remodeling often causing a mismatch in the contour of the defect.

Alloplastic filler substances include for example microdroplet, injectable liquid silicone, expanded polytetrafluoroethylene and bovine collagen-wrapped polymethylmethacrylate microspheres. These agents generally require skin testing, large-gauge needles or trocars for implantation under local anaesthetic into deep, subdermal compartments.

Examples of injectable formulations include for example one or more of: a suspension of 20% microspheres (40 μm) of polymethylmethacrylate (PMMA) in bovine collagen solution, medical grade silicone fluid (dimethylpolysiloxane), poly-L-lactic acid (L-PLA) microspheres (2-50 μm) suspended in methylcellulose, a suspension of dextran microspheres (40 μm Sephadex) in hyaluronic acid (2.5 MDa), a suspension of hydroxyethyl-methacrylate (HEMA) fragments in cross-linked hyaluronic acid, a cross-linked gel of polyacrylamide (PAAG), a suspension of polyvinylhydroxide (PVOH) microspheres (5-80 μm) in polyacrylamide gel, and a suspension of calcium hydroxylapatite microspheres (25-40 μm) in a carboxymethyl-cellulose gel.

Autologous filler substances include for example: fat, collagen or hyaluronic acid, taken from tissues and/or fluids of the subject. Tissue from a subject (including autologous fibroblast cells) can also be used as an autologous filler alone or the tissue can be combined with other autologous filler substances. Non-autologous tissue and fluids can also be used as a filler substance (ie material derived from a cadaver).

Additional useful three-dimensional matrices that can be used together with agents with peroxidase activity to produce a filler substance are constructed from materials that include biocompatible, biodegradable (or not), synthetic polymers and ECM-derived proteins and composites. Examples of ECM-derived matrices include those including crosslinked or non-crosslinked bovine, ovine, porcine, marine or human collagen fibers, crosslinked or non-crosslinked bovine, ovine, porcine, marine or human collagen fibers and glycosaminoglycans, proteoglycans or glycoproteins; collagen-hyaluronic acid foams and other collagen-hyaluronic acid combinations; combinations of collagen and chitosan; combinations of at least two of collagen (types 1-18), fibronectin, fibrin, laminin (types 1-5), decorin, elastin, perlecan, vitronectin, chondroitin sulphate, dermatan sulphate, heparin sulphate, hyaluronic acid, and keratin sulphate.

Examples of synthetic polymers (or derivatives) useful for the manufacture of three-dimensional matrices suitable as filler substances that can be used together with agents with peroxidase activity include those including Poly(esters); examples are poly(ε-caprolactone) PCL, poly(glycolic acid) PGA, poly(L-lactic acid) PLA, poly(ethylene glycol) PEG, poly(ethylene oxide) PEO. Poly(ester) derivatives include Poly(ester) copolymers, Poly(ortho esters). Poly(ester) copolymers; examples are poly(lactic acid-co-glycolic acid) PLGA, poly(D-lactic acid) PDLA, poly(L-lactic acid) PLLA, PLA-PEG, diblock PLA/PEG, triblock PLA/PEG/PLA, triblock co-polymers based on 1,5-dioxepan-2-one (DXO) and L-lactide (LLA). Poly(ortho esters); examples are 3,9-diethylidene-2,4,8,10-tetraoxaspiro[5.5]undecane (DETOSU)-based poly(orthoesters). Poly(anhydrides); examples are sebacic acid (SA), p-(carboxyphenoxy)propane (CPP), p-(carboxyphenoxy)hexane (CPH), SA/CPP copolymers, poly(fatty acid dimer-sebacic acid), poly(anhydride-imides), poly(anhydride-esters). Poly(amides); examples are poly(amino acids), poly(glutamic acid), poly(aspartic acid), poly(lactic acid-co-lysine) PLAL, poly[N-(3-hydroxypropyl)-L-glutamine], poly(iminocarbonates), tyrosine-derived poly(carbonates). Phosphorus-containing polymers; ie poly(phosphazenes), poly(dichlorophosphazenes), poly(organophosphazenes), poly[bis(carboxylatophenoxy)-phosphazene], poly(phosphoesters), poly(urethanes) including thermoplastic polyurethane elastomer synthesized using poly(hexamethylene oxide) (PHMO) and poly(dimethylsiloxane) (PDMS) macrodials. Also including Poly-L-lysine, polyethyleinimine, PAMAM dendrimers, chitosans, linear polyamidoamines, polycaprolactone (PCL), polyethylene oxide (PEO), polybutylene terephthalate (PBT), polypyrrole-containing block copolymers synthesized by RAFT polymerization and poly(ethylene)/carboxymethylcellulose (CMC) combinations. Polymers (or derivatives) can also have their internal surfaces modified by the deposition of biological molecules such as hyaluronic acid (HA), chitosan, collagen, fibronectin, laminin etc to promote biocompatibility.

Encapsulation of proteins with peroxidase activity within liposomes are described, for example, in U.S. Pat. No. 5,662,931.

Agents with peroxidase activity can be coupled to a collagen matrix for administration. Collagen matrix can release a coupled drug at a constant effective concentration. Accordingly, collagen and other ECM protein matrices can effectively be used to administer a agent with peroxidase activity in vivo, for example into a tissue or space in need thereof. In one embodiment the cross-linked collagen matrix is administered subcutaneously.

Proteins with peroxidase activity have been conjugated to antibodies for use as enzyme labels in immunoassays (eg U.S. Pat. No. 4,243,749). The conjugation of the protein having peroxidase activity to the antibody is a means to immobilise the protein having peroxidase activity to a desired site based on the antigenic specificity of the antibody.

For example a collagen specific antibody can be used to attach a protein with peroxidase activity conjugated antibody to ECM (or collagen scaffold in an implant for in vivo implantation). Alternatively, the antibody could be used to localise the protein having peroxidase activity to a particular cell type or tissue. Therefore both implantable and injectable formulations are appropriate. Peroxidases can also be covalently attached directly to scaffolds. U.S. Pat. No. 5,989,842 describes methods of conjugating HRP to antibodies and other biomolecules.

In one embodiment, agents with peroxidase activity are incorporated within the interstices of the dermal filler substance, having been contacted with the structural components of the material during its manufacture.

In another embodiment, agents with peroxidase activity are contacted with the structural components of the dermal filler substance by admixing the dermal filler material with a medium containing the agent with peroxidase activity prior to the clinical use of the filler substance. Acceptable carriers or diluents for therapeutic use are well known in the pharmaceutical art, and are described, for example, in Remington's Pharmaceutical Sciences Mack Publishing Co. (A. R. Gennaro edit. 1985). The choice of pharmaceutical carrier excipient or diluent can be selected with regard to the intended route of administration and standard pharmaceutical practice. In another embodiment, the agents with peroxidase activity are provided in powder form to be admixed with the filler substance (eg Sculptra) before solubilising the combination product with an acceptable diluent prior to clinical use.

In another embodiment, agents with peroxidase activity are admixed with autologous tissue (eg adipose tissue) prior to clinical use. The agents with peroxidase activity can be provided in an acceptable carrier of diluent or in powder form, typically containing such agents as necessary to enhance the solubility of the agents with peroxidase activity in the desired autologous tissue.

Examples of intradermal formulations suitable for injection into an animal or human including fibroblast cells are described below.

In one embodiment fibroblast cells are administered together with, in or separate to, the composition including an agent with peroxidase activity.

In one embodiment, fibroblast cells which have been incubated with an agent with peroxidase activity so as to produce ECM are administered to a soft-tissue site in need thereof.

In an alternate embodiment, an agent with peroxidase activity is optionally co-administered. For example, the cells may be treated by contacting them with an agent with peroxidase activity for a period of about 1-6 hours. Typically, the cells may be treated for a period of about 5-6 hours in vitro. Generally, the cells may be transplanted in vivo within 2-72 hours of the end of treatment with an agent with peroxidase activity. Typically, the cells are transplanted in vivo within 6-24 hours of treatment. In one embodiment, the cells are autologous to the subject. The autologous fibroblast cells may be extracted from a subject and transplanted back into that subject according to the methods described in U.S. Pat. No. 6,878,383. Additionally, an agent with peroxidase activity can be added to the fibroblast cells prior to injection (priming) to promote synthetic capacity in vivo or co-injected with the cells to provide direct stimulation of both implanted cells and dermal fibroblast cells resident within the injected area to produce ECM and promote the migration of cells to the area.

Examples of formulations including fibroblast cells stimulated with an agent with peroxidase activity include:

(i) 0.9% sterile saline (150 mM NaCl); +/− human albumin (0.01%-0.5%); +/− local anaesthetic/analgesic; examples e.g. bupivacaine hydrochloride (1.25-5 mg/ml); +/− adrenaline acid tartrate (0.0045-0.0091 mg/ml); +/− lidocaine (0.5-2%); +/− epinephrine (1:100,000-1:200,000); +/− fat, +/− collagen, +/− hyaluronic acid and fibroblast cells.

(ii) Phosphate buffered saline; +/− human albumin (0.01-0.5%); +/− local anaesthetic/analgesic; 137 mM NaCl; 2.7 mM KCl; 10 mM phosphate buffer; 150 mM NaCl; 150 mM NaH₂PO₄/Na₂HPO₄; +/− fat, +/− collagen, +/− hyaluronic acid and fibroblast cells.

(iii) Phosphate-Citrate buffer (50 mM) +/− sodium perborate (0.03%); +/− human albumin (0.01-0.5%); +/− local anaesthetic/analgesic; +/− fat, +/− collagen, +/− hyaluronic acid and fibroblast cells.

(iv) Krebs-Ringer's solution containing NaCl 118.1 mM; KCl 3.4 mM; CaCl₂ 2.5 mM; MgSO₄ 0.8 mM; KH₂PO₄ 1.2 m; NaHCO₃ 25.0 mM; Glucose 11.1 mM; +/− fat, +/− collagen, +/− hyaluronic acid and fibroblast cells.

Formulations containing fibroblasts may also further include one or more of the following: a steroidal anti-inflammatory drug (corticosteroid), a calcineurin inhibitor (eg pimecrolimus, tacrolimus), a phosphodiesterase inhibitors, an anti-histamine, an anti-microbial agent, an antibiotic, an antibacterial agent, a ceremide, a growth factor (eg transforming growth factors β1-3, platelet derived growth factor, fibroblast growth factor, insulin-like growth factors I & II, epidermal growth factor, keratinocyte growth factor, nerve growth factor), a mitogenic agent, a matrix metalloproteinase inhibitor (eg TIMP's, Batimastat, Marimastat, and matlystatin B), a protease inhibitor, an angiogenic promoter, a chemotactic agent, a ECM protein, tretinoin (Vitamin A), a antioxidant (vitamins E and C), a plant cytokinin (kinerase), a copper-peptide complexes as well as numerous plant, animal and mineral extracts (ie coal tar extract).

In one embodiment, formulations of the present invention include a combination with a steroidal anti-inflammatory drug. Another composition includes a calcineurin inhibitor. Another composition includes an anti-histamine. Another composition includes an anti-microbial agent. Another composition includes a growth factor. Another composition includes a protease inhibitor. Another composition includes an angiogenic promoter. Another composition includes a chemotactic agent.

As described previously herein, in one embodiment the present invention may be used for augmentation of soft tissue.

In this regard, the present studies demonstrate that agents with peroxidase activity promote the generation of new fibroblast-derived tissue within and around materials commonly used as dermal augmentation devices for cosmetic and structural enhancement.

As such, compositions including agents with peroxidase activity and dermal filler agents may be used to provide an improved fibroblastic response and a greater ECM response than that provided by the fillers alone.

In addition, the combination of an agent with peroxidase activity with filler substances has the benefit of providing extended and controlled, natural volume expansion. That is, the dermal filler substance provides the short-term augmentation effect whilst the longer-term volume correction is provided by the patients own regenerative capacity as stimulated by the agents with peroxidase activity. The degree of volume expansion maybe controlled by the dose of the agent having peroxidase administered together with optimisation of the particle size of the filler substance used. The initiation of a regenerative response within and about the filler substance would increase the overall longevity of the implant and promote a longer lasting cosmetic effect. In addition, the integration of the dermal filler substance with the surrounding tissue stimulated by the agents with peroxidase activity means the filler substance is less likely to migrate, thus providing a more stable augmentation result. This will enable dermal filler substances combined with agents with peroxidase activity to be effective for example in mobile areas of the face and neck where the risk of implant migration currently restricts their use. In addition, the improved volume expansion and filler longevity provided by combining filler substances with agents with peroxidase activity provides a much more cost effective treatment for patients desiring cosmetic and structural dermal augmentation outcomes.

The examples also demonstrate agents with peroxidase activity are ideal adjuvants for products (eg Macrolane) designed to correct body asymmetries after liposuction, scarring or for breast augmentation. In addition, agents with peroxidase activity may be used as additives to periurethral and generalised sphincter bulking agents to enhance and prolong the action of the bulking agent.

The present studies also demonstrate that agents with peroxidase activity can increase ECM component production when injected into human skin. The compositions of the invention can therefore be employed for cosmetic improvement by increasing the amount of collagen in the skin as a stand-alone injectable product. As such, the compositions of the invention can be used in most applications where cosmetic or reconstructive skin implantation and augmentation is conventionally used by way of an injectable filler substance (such as heterologous and alloplastic filler substances), but with the additional benefit of ECM being locally secreted and deposited into a fully cross linked matrix which provides a more durable matrix due to its cross linking, compared to non-cross linked exogenous collagen which is prone to rapid degradation by matrix metalloproteinases and other biological processes after implantation. The ability of the compositions to induce cells to increase the amount of ECM produced means that the risk of allergic reactions and disease that may occur with the use of exogenous heterologous filler substances (such as bovine collagen) can be avoided or at least minimised. However there may also be applications where both immediate and long term tissue augmentation/filling is required and in this instance the compositions of the invention may also include heterologous and alloplastic filler substances (including collagen, hyaluronic acid and other ECM proteins) in addition to the agent with peroxidase activity.

The present invention can also be employed in most applications where collagen or other filler substances are conventionally used for implantation and augmentation. Also, the present invention can be used where the endogenous production of ECM provides the additional benefit of the ECM proteins being locally excreted and deposited into a naturally formed matrix which provides a more durable matrix due to its normal cell-matrix, matrix-matrix interactions and cross linking, compared to non-cross linked exogenous collagen and other filler substances (eg hyaluronic acid) which are prone to rapid degradation by matrix metalloproteinases and other biological processes after implantation. The ability of the compositions of the present invention to induce cells to increase the amount of ECM produced including collagen means that the risk of allergic reactions and disease that may occur with the use of an exogenous ECM component can be reduced. There may also be applications where both immediate and long term tissue augmentation/filling is required and in this instance the compositions of the present invention may also include collagen, other ECM proteins, autologous, heterologous or alloplastic filler substances in addition to the protein with peroxidase activity. In addition a foreign source of proteins with peroxidase activity may be avoided where desired, by obtaining autologous proteins with peroxidase activity from a subject's cells or tissues for re-administration to that subject.

The compositions may be administered to a site that is selected to be most suitable for augmentation, for example a pit in a soft tissue can be filled directly or can be filled by implanting the filler substance underneath the area of the soft tissue to provide the same filling effect. Furthermore the compositions of the present invention can be used for creating soft tissue blebs. Applications of compositions of the present invention include facial contouring or conditions that may be associated with ECM loss leading to volume depletion and soft tissue contour defect (frown or glabellar line, acne scars, chicken pox scars, cheek depressions, vertical or perioral lip lines, marionette lines or oral commissures, worry or forehead lines, crow's feet or periorbital lines, deep smile lines or nasolabial folds, rhytides, smile lines, facial scars, lips and the like and where effective restitution of skin structure and the smooth appearance of skin is desired); periurethral injection including injection into the submucosa of the urethra along the urethra, at or around the urethral-bladder junction to the external sphincter; ureteral injection for the prevention of urinary reflux; injection into the tissues of the gastrointestinal tract for the bulking of tissue to prevent reflux; to aid in sphincter muscle coaptation, internal or external, and for coaptation of an enlarged lumen; injection into anatomical ducts to temporarily plug the outlet to prevent reflux or infection propagation; larynx rehabilitation after surgery or atrophy; and any other soft tissue which can be augmented for cosmetic or therapeutic affect. Other application of agents with peroxidase activity include the repair of soft tissue tears and where deep sutures may be used or in the integration or bonding of implanted or superficially attached soft tissues such as for tissue or synthetic grafting and where abdominal wall and back skin needs reattachment after major flap harvest operations. Agents with peroxidase activity can also be used to promote dead space obliteration after elevation of facial, brow or neck skin with rejuvenation surgery.

Further, agents with peroxidase activity may be injected into the skin tissue to augment the skin tissue to repair or correct congenital anomalies, acquired defects, or cosmetic defects. Examples of such conditions are congenital anomalies such as hemifacial microsomia, malar and zygomatic hypoplasia, unilateral mammary hypoplasia, pectus excavatum, pectoralis agenesis (Poland's anomaly) and velopharyngeal incompetence secondary to cleft palate repair or submucous cleft palate (as a retropharyngeal implant); acquired defects (post traumatic, post surgical, post infectious) such as depressed scars, subcutaneous atrophy (e.g., secondary to discoid lupus erythematosus), keratotic lesions, acne pitting of the face, linear scleroderma with subcutaneous atrophy, saddle-nose deformity, Romberg's disease and unilateral vocal cord paralysis; and cosmetic defects such as glabellar frown lines, deep nasolabial creases, circum-oral geographical wrinkles, sunken cheeks and mammary hypoplasia. Other forms of augmentation that agents with peroxidase activity may be used for include the following: rhinoplasty, malar and submalar augmentation, chin augmentation, tear-trough contouring, liposuction defects, orbital cavity augmentation, oral soft-tissue ridge augmentation, nipple augmentation and phalloplasty.

Agents with peroxidase activity may be administered by injection or with a needle e.g. a wide gauge needle or incorporated within a variety of devices.

It is also envisioned that agents with peroxidase activity can be sold in the form of a kit including a device containing the agent with peroxidase activity, the device having an outlet for said agents with peroxidase activity, an ejector for expelling the agents with peroxidase activity and a hollow tubular member fitted to the outlet for administering the agents with peroxidase activity into an animal.

The following provides generally a description of various applications of using agents with peroxidase activity to promote tissue generation, tissue regeneration, tissue repair and tissue support when the agent is incorporated within a substrate to generate replacement tissue in vitro for application in vivo, or for direct application of the substrate in vivo (ie to act as a tissue regeneration template or delivery device to promote repair of a tissue in need thereof).

In the present studies it has been found that fibroblast cells stimulated with an agent with peroxidase activity populated the interstices of a porous collagen-based three dimensional scaffold much quicker and more effectively than untreated cells when seeded thereon in vitro. In addition, the fibroblast cells that migrated into the structure of the three-dimensional scaffold in response to being stimulated by an agent with peroxidase activity, were also stimulated to produce increased amounts of ECM by the agent with peroxidase activity. The increased amounts of ECM produced by the fibroblast cells were elaborated as increased soluble levels of ECM in the media bathing the three-dimensional scaffold, and also as increased deposition of cell-associated ECM within and about the interstices of the scaffold. The ECM deposited by the fibroblast cells was observed to contact both the cells and the structural elements of the three-dimensional scaffold, thus enhancing the conversion of the scaffold to a more complete and useful tissue substitute. In addition, the present studies have shown that the present invention may be used to promote the production of ECM by fibroblast cells encapsulated in a synthetic hydrogel matrix and contacted by a agent with peroxidase activity as compared to cells not treated with an agent with peroxidase activity but maintained in normal, supplemented culture media. It has also been found in the present studies that the present invention may be used to increase the speed by which fibroblast cells populate the three-dimensional scaffold by at least seven to fourteen days and similarly, the present invention can be used to increase the speed by which fibroblast cells fill the interstices of the three-dimensional scaffold with ECM by at least seven to fourteen days. It will be appreciated that there are economic and clinical benefits arising from the ability to reduce the time required to convert a three-dimensional scaffold into usable replacement tissue in vitro.

In one embodiment, a scaffold can be contacted with an agent with peroxidase activity during manufacture. Alternatively, a preformed three-dimensional scaffold is contacted with an agent with peroxidase activity for a time and under conditions necessary for the agent with peroxidase activity to bind to the structural components of the scaffold. For example, the agent with peroxidase activity may be contacted with the scaffold for up to 1 hour, or the agent with peroxidase activity may be contacted with the scaffold for up to 16 hours, or the agent with peroxidase activity may be contacted with the scaffold for the duration of the culture time required to generate a substitute tissue. The agent with peroxidase activity may be contacted with the scaffold once the cells have been seeded thereon, or the agent with peroxidase activity may be contacted with the scaffold prior to the seeding of the cells thereon, or the agent with peroxidase activity may be contacted with the scaffold prior to and after seeding of the cells thereon. This may be accomplished, for example, by the agent with peroxidase activity in a medium suitable for contacting the agent with peroxidase activity with the structural components of the scaffold (eg water, a physiological salt solution, a culture medium). The contact between the agent with peroxidase activity and scaffold components can be direct or indirect and can be permanent or non-permanent. In another embodiment, the agent with peroxidase activity is contacted with the structural components of the scaffold during the manufacture of the scaffold and thus the agent with peroxidase activity becomes part of the complete tissue regeneration scaffold.

Various methods for producing three dimensional cell scaffolds useful for generating replacement tissue in vitro for transplantation in vivo are described in the art. For example, U.S. Pat. No. 5,759,830 describes a cell-scaffold composition prepared in vitro for use to produce functional organ tissue in vivo. Cells having a desired function are grown on a polymer scaffolding using cell culture techniques followed by transfer of the polymer-cell scaffold into a patient at a site appropriate for attachment, growth and function. Nutrients and growth factors are supplied during cell culture allowing for attachment, survival or growth as needed. U.S. Pat. No. 5,759,830 discloses that the material for forming the matrix or support structure is a biodegradable artificial polymer, and that cells of one or more types can be selected and grown on the matrix. The matrix structure and the length of time and conditions under which the cells are cultured in vitro are determined on an individual basis for each type of cell. The cells are initially cultured using techniques known to those skilled in the art of tissue culture. According to the present invention, fibroblast cells are stimulated to produce ECM proteins by an agent with peroxidase activity which are secreted and deposited into the interstices of the 3D-scaffold surrounding the cell. Once the cells have been stimulated so as to cover or partially cover the scaffold with sufficient ECM, the scaffold can then be implanted into an animal in need thereof at a site appropriate for attachment, growth and function.

U.S. Pat. No. 5,962,325 for example describes a stromal cell matrix formed onto which cell specific types are seeded for forming specific tissue types. Stromal cells are inoculated and grown on three-dimensional scaffolds. The stromal cells and proteins naturally produced by the stromal cells attach to and substantially envelope the framework composed of a biocompatible non-living material formed into a three-dimensional structure. Fibroblast cells can be stimulated according to the present invention to produce ECM proteins using exogenously added agents with peroxidase activity.

The present invention is also useful when used for promoting the development of a dermal component of a bioengineered skin substitute prior to seeding of the newly formed dermis “equivalent” with a layer of keratinocytes (epidermal cells). The “skin substitute” (also referred to as “skin equivalent”) as a whole becomes the clinically useful product—therefore the agents with peroxidase activity are useful in the manufacture of these skin substitutes.

As previously described herein, agents with peroxidase activity may be delivered within the interstices of a supporting scaffold or supplied by addition to a culture medium bathing a scaffold. Agents with peroxidase activity can also be covalently attached directly to scaffolds during the manufacture of the scaffold. U.S. Pat. No. 5,989,842 describes methods of conjugating HRP to antibodies and other biomolecules. The cells and the agents with peroxidase activity may be heterologous to the subject to be treated, or where the skin substitute is to be generated using the patients own cells, the agents with peroxidase activity may be heterologous or autologous to the subject to be treated.

An example of a skin substitute that relies on the formation of a fibroblast-derived ECM within a three-dimensional scaffold is DERMAGRAFT® (see U.S. Pat. Nos. 4,963,489, 5,266,480, 5,443,950). Apligraf® is another example of a skin substitute that relies on the formation of fibroblast-derived ECM to augment the primary scaffold that consists of bovine type I collagen on a semi-permeable membrane.

The present studies demonstrate that agents with peroxidase activity enhance the migration of both adult fibroblast cells and HFF cells into a three-dimensional framework or scaffold and promote the population of the interstices of the scaffold by the fibroblast cells. The examples show that after seven days of culture, the number of cells populating a three-dimensional framework or scaffold treated with agents with peroxidase activity are at least equal to, but generally greater than, the number of cells populating a three-dimensional framework or scaffold after fourteen days of culture without the addition of agents with peroxidase activity. These results demonstrate that agents with peroxidase activity decrease by at least 7 days the time it takes for the scaffold to become populated by cells to the same level observed when current, standard culture conditions are used. As such, agents with peroxidase activity may be used in preparing three dimensional tissues for clinical and experimental use when using scaffolds and frameworks (generated from naturally occurring proteins or biocompatible polymers) as regeneration templates. Reducing the time it takes to manufacture heterologous three dimensional tissues (using HFF cells for example) improves their commercial viability (due to reduced manufacturing costs etc). Major clinical benefits for patients can be provided when the time taken to generate autologous three dimensional replacement tissue can be markedly reduced. For example, it will be appreciated that reducing the time it takes to generate an autologous skin equivalent from 3 weeks to 2 weeks can provide significant benefits in terms of the mortality, morbidity and healing outcome of a patient with severe burns.

The present invention may also be used to provide a replacement tissue that is less cellular and can be cultured more quickly compared to conventional techniques. A skin substitute made by the methods of the present invention may have a fibroblastic cellular content more consistent with normal skin. Advantageously, the present invention may be used to reduce the need for FBS to culture cells. Also the use of agents with peroxidase activity in the culture of cells or the production of replacement tissue may reduce the cost of the process compared to a process which does not employ the methods or compositions of the present invention.

The present invention may also be used to provide a fibroblast populated and ECM-enriched three dimensional scaffold or framework that is more receptive to the inoculation of additional cells such as tissue specific cells (ie vascular endothelial cells, hair follicle cells, sebaceous gland cells, sweat gland cells etc) to create a more functional regenerated tissue. The cells chosen for inoculation will depend upon the tissue to be cultured, which may include skin, bone marrow, liver, pancreas, kidney, neurological tissue, adrenal gland, mucosal epithelium, and smooth muscle to name but a few. In general, this inoculum should include the stem cell for that tissue; i.e., those cells which generate new cells that will mature into the specialised cells that form the various components of the tissue.

The products produced according to the present invention may also be stronger and have increased resistance to shear and maceration than similar products.

Further, the relative increase in the amount of ECM present in the skin substitute may provide structural support for the development of new blood vessels with the capacity to promote vascularisation, ie support a vascular plexus, and nutrient delivery to the skin substitute when it is grafted or transplanted onto the patient. It will be appreciated that this function would greatly improve the successful engraftment of the replacement tissue and thus greatly enhance the desired clinical benefit to the patient.

Agents with peroxidase activity that remain within the skin substitute at the time of grafting or transplantation onto the patient may promote integration of the skin substitute with the wound bed and surrounding skin by stimulating the migration of the patients own cells into the skin substitute. Agents with peroxidase activity that elute out of the skin substitute as demonstrated by present studies may stimulate additional cells to migrate to the site of grafting or transplantation and to produce ECM, thus augmenting the healing process and promoting the integration and survival of the grafted or transplanted skin substitute.

In addition, the present studies demonstrate fibroblast cells encapsulated within a synthetic hydrogel matrix and contacted with agents with peroxidase activity, greatly increase the amount of ECM they produce. Slowly polymerizing, biocompatible, biodegradable hydrogels are described for example in U.S. Pat. No. 5,70,854. The gels are demonstrated to be useful as a means of delivering large numbers of isolated cells into a patient to create replacement tissue. The gels promote engraftment and provide three dimensional templates for new cell growth. Cells are suspended in a hydrogel solution and in one embodiment the solution is poured or injected into a mould having a desired anatomical shape, then hardened to form a matrix having cells dispersed therein which can be implanted into a patient. The polymeric matrix can be combined with humoral factors to promote cell survival and host acceptance following transplantation and engraftment. The polymeric matrix can be combined with an agent with peroxidase activity to stimulate the cells to produce an extracellular matrix. The agent with peroxidase activity can be mixed in a slow release form. Alternatively the hydrogel can be modified to bind the agent with peroxidase activity prior to combination with an isolated cell suspension. In another example, agents with peroxidase activity may be incorporated into biocompatible polyalkymide hydrogels such as Bio-Alcamid.

Additional three-dimensional cell scaffold templates that can be used together with agents with peroxidase activity for the generation of replacement tissue can be constructed from materials that include biocompatible, biodegradable (or not), synthetic polymers and ECM-derived proteins and composites. Examples of ECM-derived scaffolds include those including crosslinked or non-crosslinked bovine, ovine, porcine, marine or human collagen fibers, crosslinked or non-crosslinked bovine, ovine, porcine, marine or human collagen fibers and glycosaminoglycans, proteoglycans or glycoproteins; collagen-hyaluronic acid foams and other collagen-hyaluronic acid combinations; combinations of collagen and chitosan; combinations of at least two of collagen (types 1-18), fibronectin, laminin (types 1-5), decorin, elastin, perlecan, vitronectin, chondroitin sulphate, dermatan sulphate, heparin sulphate, hyaluronic acid, and keratin sulphate.

Examples of synthetic polymers (or derivatives) useful for the manufacture of three-dimensional cell scaffolds that can be used together with agents with peroxidase activity include those including Poly(esters); examples are poly(ε-caprolactone) PCL, poly(glycolic acid) PGA, poly(L-lactic acid) PLA, poly(ethylene glycol) PEG, poly(ethylene oxide) PEO. Poly(ester) derivatives include Poly(ester) copolymers, Poly(ortho esters). Poly(ester) copolymers; examples are poly(lactic acid-co-glycolic acid) PLGA, poly(D-lactic acid) PDLA, poly(L-lactic acid) PLLA, PLA-PEG, diblock PLA/PEG, triblock PLA/PEG/PLA, triblock co-polymers based on 1,5-dioxepan-2-one (DXO) and L-lactide (LLA). Poly(ortho esters); examples are 3,9-diethylidene-2,4,8,10-tetraoxaspiro[5.5 ]undecane(DETOSU)-based poly(orthoesters). Poly(anhydrides); examples are sebacic acid (SA), p-(carboxyphenoxy)propane (CPP), p-(carboxyphenoxy)hexane (CPH), SA/CPP copolymers, poly(fatty acid dimer-sebacic acid), poly(anhydride-imides), poly(anhydride-esters). Poly(amides); examples are poly(amino acids), poly(glutamic acid), poly(aspartic acid), poly(lactic acid-co-lysine)PLAL, poly[N-(3-hydroxypropyl)-L-glutamine], poly(iminocarbonates), tyrosine-derived poly(carbonates). Phosphorus-containing polymers; ie poly(phosphazenes), poly(dichlorophosphazenes), poly(organophosphazenes), poly[bis(carboxylatophenoxy)-phosphazene], poly(phosphoesters), poly(urethanes) including thermoplastic polyurethane elastomer synthesized using poly(hexamethylene oxide) (PHMO) and poly(dimethylsiloxane) (PDMS) macrodials. Also including Poly-L-lysine, polyethyleinimine, PAMAM dendrimers, chitosans, linear polyamidoamines, polycaprolactone (PCL), polyethylene oxide (PEO), polybutylene terephthalate (PBT), polypyrrole-containing block copolymers synthesized by RAFT polymerization and poly(ethylene)/carboxymethylcellulose (CMC) combinations. Polymers (or derivatives) can also have their internal surfaces modified by the deposition of biological molecules such as hyaluronic acid (HA), chitosan, collagen, fibronectin, laminin etc to promote biocompatibility.

In addition to the clinical use of replacement tissue generated in vitro, bioengineered tissue, and in an embodiment substitute skin tissue, can be used as alternatives to animal testing for the many thousands of chemical additives used in human skin products. They can also be used for investigating cell-cell and cell-ECM interactions, skin barrier penetration, wound healing, angiogenesis, regulation of pigmentation, skin contraction and investigation of skin diseases such as melanoma invasion, psoriasis and skin blistering disorders. Most existing commercial three-dimensional models of skin contain mainly keratinocytes and need to be improved to include fibroblast cells and fibroblast-derived ECM.

In addition to the finding that fibroblast cells stimulated with an agent with peroxidase activity populated the interstices of a porous collagen-based three dimensional scaffold much quicker and more effectively than untreated cells when seeded thereon in vitro, it has been found in the present studies that including agents with peroxidase activity within a three-dimensional ECM-derived scaffold transplanted in vivo, promotes the migration of fibroblast cells into the structure of the three-dimensional scaffold and the production of ECM by the fibroblast cells.

Together, these two demonstrate that agents with peroxidase activity can greatly enhance the function of tissue regeneration templates and devices such as INTEGRA® Dermal Regeneration Template that are used clinically to replace lost tissue and to aid healing and repair. For example, INTEGRA® is applied to wounds such as burns to facilitate the formation of a neodermis, with the collagen/glycosaminoglycan scaffold acting as a template for the infiltration of fibroblast cells, macrophages and capillaries. The neodermis generally takes between 14 and 21 days to develop after the application of the INTEGRA® and ambulation, physical therapy and range of motion exercises are delayed until at least 7 to 10 days after application of the INTEGRA® to ensure mechanical dislodgement of the INTEGRA® is avoided during the generation of the neodermis (which would lead to graft failure and potential wound contamination). The incorporation of agents with peroxidase activity into a scaffold or framework prepared as a tissue regeneration template, either during the manufacture of the template and/or at the time of application of the template to the wound, and/or at reasonable intervals after application of the template to the wound, may be used for improving the function of the template. For example, the addition of agents with peroxidase activity to a regeneration template such as INTEGRA®, would reduce the time taken to generate new neodermis due to the ability of the agents with peroxidase activity to stimulate the infiltration and population of the template by fibroblast cells from the wound bed and the generation of ECM within the interstices of the template. The generation of new neodermis is dependent on the ability of new blood vessels to grow into the dermal regeneration template, and it will be appreciated that this function is greatly enhanced by the formation of fibroblast-associated ECM such as that stimulated by agents with peroxidase activity. Reducing the time required to generate the new neodermis has benefits as it means subsequent treatments required for the complete healing of wounds such as application of an epidermal autograft can be performed much sooner, thus providing the patient with significant improvements in structural, functional and cosmetic wound healing outcomes. The addition of agents with peroxidase activity would be a suitable adjuvant for wounds indicated for treatment with dermal regeneration templates such as partial and full-thickness wounds, pressure ulcers, venous ulcers, diabetic ulcers, chronic vascular ulcers, surgical wounds (donor sites/grafts, post-Moh's surgery, post-laser surgery, podiatric, dehisced wounds), trauma wounds (abrasions, lacerations, second and third-degree burns, skin tears) and draining wounds.

As previously described herein, agents with peroxidase activity may be delivered within the interstices of the regeneration template, having been contacted with the structural components of the template during its manufacture.

In one embodiment, the agents with peroxidase activity are contacted with the structural components of the template by incubating the template in a medium containing the agents with peroxidase activity, for example, for up to 16 hours prior to the clinical use of the template. Acceptable carriers or diluents for therapeutic use are well known in the pharmaceutical art, and are described for example in Remington's Pharmaceutical Sciences Mack Publishing Co. (A.R. Gennaro edit. 1985). The choice of pharmaceutical carrier excipient or diluent can be selected with regard to the intended route of administration and standard pharmaceutical practice. The medium containing agents with peroxidase activity may be a “wash solution” or a “rehydration solution” where these solutions are required for the preparation of the dermal regeneration template prior to the clinical use of the template.

As previously discussed herein, the agents with peroxidase activity may be delivered within the interstices of the regeneration template as aforementioned, and additional agents with peroxidase activity delivered to the site of grafting or transplantion prior to or at suitable times after the grafting or transplantation of the regeneration template.

As previously described herein, the present invention may also be used to improve the integration of the regeneration template with the wound bed and surrounding tissue by stimulating the migration of the patients own cells into the template and the production of endogenous ECM such that the ECM generated provides a structural support for the development of new blood vessels with the capacity to promote vascularisation, ie support a vascular plexus, and nutrient delivery to the regenerating tissue.

As previously described herein, the present invention may be used to improve the integration of the regeneration template as aforementioned with the additional benefit that the agents with peroxidase activity will elute out of the regeneration template as demonstrated by the present studies, and stimulate additional cells to migrate to the site of grafting or transplantation and to produce ECM, thus augmenting the healing process and further promoting the integration and survival of the grafted or transplanted tissue regeneration template.

The present invention may also be used to improve the clinical outcome by decreasing the time until subsequent surgical procedures such as application of epidermal autografts can be performed, improving their efficacy, reducing wound contamination, reducing scarring and enhancing the cosmetic appearance and the like.

The present invention may also provide stronger regenerated tissue with increased resistance to shear and maceration and enable patients to improve their range of movement when regeneration templates are grafted or transplanted over joints or mobile areas.

Useful skin regeneration templates that can be used with agents with peroxidase acivity can be temporary or permanent. Temporary skin regeneration templates include for example cutaneous allografts (such as human cadaver split-thickness skin), cutaneous xenografts (such as porcine dermis), amniotic membranes (such as human amniotic membrane obtained from placenta), porcine small intestinal submucosa (such as Oasis®), and composite synthetic-biological collagen based dermal analogs (such as Biobrane®; a bilayer membrane consisting of an outer silicone film attached to a 3-D nylon mesh containing bovine collagen type I; or Transcyte®; a bilayer membrane consisting of an outer silicone film and a dermal analog layered with human neonatal foreskin fibroblast cells and containing secreted proteins such as collagen I , fibronectin and glycosaminoglycans).

Permanent skin regeneration templates include for example bilayer structures with a biologic dermal analog and a either synthetic or biologic epidermal analogue (such as Apligraf; a collagen matrix seeded with human neonatal fibroblast cells and keratinocytes; and OrCel; a collagen sponge seeded with human neonatal fibroblast cells and keratinocytes), skin components containing epidermal cells alone (such as Epicel; cultured autologous keratinocytes), or dermis alone (such as Alloderm; acellular dermis derived from processed allograft [cadaver] skin) or dermal regeneration templates (such as INTEGRA®; silicone outer layer on a collagen/glycosaminoglycan dermal matrix).

Additional useful three-dimensional tissue regeneration templates that can be used together with agents with peroxidase activity can be constructed from materials that include biocompatible, biodegradable (or not), synthetic polymers and ECM-derived proteins and composites. Examples of ECM-derived templates include those including crosslinked or non-crosslinked bovine, ovine, porcine, marine or human collagen fibers, crosslinked or non-crosslinked bovine, ovine, porcine, marine or human collagen fibers and glycosaminoglycans, proteoglycans or glycoproteins; collagen-hyaluronic acid foams and other collagen-hyaluronic acid combinations; combinations of collagen and chitosan; combinations of at least two of collagen (types 1-18), fibronectin, laminin (types 1-5), decorin, elastin, perlecan, vitronectin, chondroitin sulphate, dermatan sulphate, heparin sulphate, hyaluronic acid, and keratin sulphate.

Examples of synthetic polymers (or derivatives) useful for the manufacture of three-dimensional tissue regeneration templates that can be used together with agents with peroxidase activity include those including Poly(esters); examples are poly(ε-caprolactone) PCL, poly(glycolic acid) PGA, poly(L-lactic acid) PLA, poly(ethylene glycol) PEG, poly(ethylene oxide) PEO. Poly(ester) derivatives include Poly(ester) copolymers, Poly(ortho esters). Poly(ester) copolymers; examples are poly(lactic acid-co-glycolic acid) PLGA, poly(D-lactic acid) PDLA, poly(L-lactic acid) PLLA, PLA-PEG, diblock PLA/PEG, triblock PLA/PEG/PLA, triblock co-polymers based on 1,5-dioxepan-2-one (DXO) and L-lactide (LLA). Poly(ortho esters); examples are 3,9-diethylidene-2,4,8,10-tetraoxaspiro[5.5 ]undecane(DETOSU)-based poly(orthoesters). Poly(anhydrides); examples are sebacic acid (SA), p-(carboxyphenoxy)propane (CPP), p-(carboxyphenoxy)hexane (CPH), SA/CPP copolymers, poly(fatty acid dimer-sebacic acid), poly(anhydride-imides), poly(anhydride-esters). Poly(amides); examples are poly(amino acids), poly(glutamic acid), poly(aspartic acid), poly(lactic acid-co-lysine)PLAL, poly[N-(3-hydroxypropyl)-L-glutamine], poly(iminocarbonates), tyrosine-derived poly(carbonates). Phosphorus-containing polymers; ie poly(phosphazenes), poly(dichlorophosphazenes), poly(organophosphazenes), poly[bis(carboxylatophenoxy)-phosphazene], poly(phosphoesters), poly(urethanes) including thermoplastic polyurethane elastomer synthesized using poly(hexamethylene oxide) (PHMO) and poly(dimethylsiloxane) (PDMS) macrodials. Also including Poly-L-lysine, polyethyleinimine, PAMAM dendrimers, chitosans, linear polyamidoamines, polycaprolactone (PCL), polyethylene oxide (PEO), polybutylene terephthalate (PBT), polypyrrole-containing block copolymers synthesized by RAFT polymerization and poly(ethylene)/carboxymethylcellulose (CMC) combinations. Polymers (or derivatives) can also have their internal surfaces modified by the deposition of biological molecules such as hyaluronic acid (HA), chitosan, collagen, fibronectin, laminin etc to promote biocompatibility.

In addition to the finding that stimulation with an agent with peroxidase activity promotes the migration of fibroblast cells into the structure of a three-dimensional scaffold and the production of ECM by the fibroblast cells in vivo, the present sudies also demonstrate that agents with peroxidase activity stimulate a “fibrogenic response” within the dermal layer of the skin. In addition, the present studies demonstrate that agents with peroxidase activity absorbed into a collagen-based three-dimensional matrix can elute out of the matrix over time and stimulate ECM production by fibroblast cells within the close proximity of the matrix. In addition, the present studies demonstrate that collagen fibers generated by fibroblast cells in response to agents with peroxidase activity are positioned in an orderly, parallel fashion in the cell-associated ECM, unlike the disorganised, cross-hatched or swirled appearance associated with unwanted scar formation.

Together, these findings demonstrate that agents with peroxidase activity can greatly enhance the wound healing function of dressings that are used clinically to aid healing and repair. For example, the “fibrogenic response” is a well known aspect of wound healing that promotes the formation of granulation tissue within a wound. The fibrogenic response fills the wound space with new tissue (principally fibroblast cells together with new ECM produced by the fibroblast cells) which supports neovascularisation leading to the formation of a new vascular network within the healing tissue. Filling the wound space with new tissue, particularly ECM, and more particularly collagen molecules, also enables, supports and directs the reepithelialisation of the wound by epidermal keratinocytes, thus leading to restoration of the skin barrier function and clinically defined healing. In addition, filling the wound space with organised ECM may provide the added advantage of limiting the development of disorganised scar tissue and providing improved structural, functional and cosmetic outcomes.

The present studies demonstrate that when the dermal layer of skin of an animal is contacted with agents with peroxidase activity, a dose-dependent fibrogenic response is induced. The fibrogenic response is elucidated by histological evaluation of the fibroblastic response, whereby fibroblast cells accumulate within the localised area of dermis contacted with agents with peroxidase activity, together with the histological evaluation of the amount of new ECM deposited by the accumulated fibroblast cells.

Overall, the combination of fibroblast accumulation and new ECM production (the fibrogenic response) generates new granulation tissue with the present studies demonstrating this occurs within three days of the dermis being contacted with agents with peroxidase activity.

Wound healing is generally regarded as a temporally controlled process that progresses through sequential but overlapping stages. The principle function of a wound dressing is to provide an optimum healing environment, consistent with the stage of wound healing. A moist environment is generally considered advantageous for the formation of new granulation tissue. In accordance with the present invention, an even more advantageous environment can be achieved by the addition of agents with peroxidase activity to wound dressings used during the stages of wound healing whereby a fibrogenic response and the formation of granulation tissue is desired. The addition of agents with peroxidase activity would be a suitable adjuvant for wounds indicated for treatment with wound dressings such as partial and full-thickness wounds, pressure ulcers, venous ulcers, diabetic ulcers, chronic vascular ulcers, contaminated wounds, surgical wounds (donor sites/grafts, post-Moh's surgery, post-laser surgery, podiatric, dehisced wounds), trauma wounds (abrasions, lacerations, second and third-degree burns, skin tears and cracked skin) and draining wounds.

As previously described herein, agents with peroxidase activity may be delivered within the interstices, gel, sheet, membrane or paste of the wound dressing, having been contacted with the structural components of the wound dressing during its manufacture.

As previously described herein, the agents with peroxidase activity may be contacted with the structural components of the wound dressing by incubating or moistening the dressing in a medium containing the agents with peroxidase activity prior to the clinical use of the dressing. Acceptable carriers or diluents for therapeutic use are well known in the pharmaceutical art, and are described for example in Remington's Pharmaceutical Sciences Mack Publishing Co. (A.R. Gennaro edit. 1985). The choice of pharmaceutical carrier, excipient or diluent can be selected with regard to the intended route of administration and standard pharmaceutical practice. In one embodiment, a medium containing agents with peroxidase activity is provided together with a dressing in a dressing pack to aid the clinical use of said combination.

In another embodiment, the agents with peroxidase activity may be delivered within the interstices, gel, sheet, membrane or paste of the wound dressing as aforementioned, and additional agents with peroxidase activity delivered to the site of the wound prior to or at suitable times after the application of the dressing to the wound for as long as necessary to achieve the desired result.

In another embodiment, the present invention may improve the healing of the wound by stimulating the migration of the patients own cells into the wounded area and the production of endogenous ECM such that new granulation tissue is generated.

The present invention may also be used to improve the healing of the wound as aforementioned with the additional benefit that the agents with peroxidase activity will elute out of the wound dressing as demonstrated by the examples herein and stimulate additional cells to migrate to the site of the wound and to produce ECM.

In one embodiment, the present invention may be used to improve the clinical outcome by decreasing the time required to heal the wound, thus decreasing patient pain and discomfort, reducing wound contamination, reducing scarring and enhancing the cosmetic appearance and the like.

Under some circumstances, the present invention may also provide stronger, more durable healed tissue with increased structure and function and increased resistance to shear and maceration.

Useful wound dressings that can be used with agents with peroxidase activity include:

Collagen dressings: gels, membranes, pads, particles, pastes, powders, sponges, sheets or solutions derived from bovine, porcine or avian sources. Dressings maybe composites such as Promogran®, a dressing made by combining animal collagen (55%) with oxidized regenerated cellulose (45%), or Fibrocol Plus®, a dressing made by combining animal collagen (90%) with an alginate (10%).

Foam dressings: sheets and other shapes of foamed polymer solutions (most commonly polyurethane) with small, open cells capable of holding fluids. They maybe impregnated or layered in combination with other materials.

Biological and biosynthetic dressings: gels, solutions or semi-permeable sheets derived from natural sources such as glycosaminoglycan matrix (ie Humatrix® Microclysmic Gel) and irradiated human skin (ie GammaGraft™)

Hydrocolloid dressings: wafers, powders or pastes composed of gelatin, pectin or carboxymethylcellulose.

Amorphous Hydrogel dressings: formulations of water, polymers and other ingredients with no shape (often in gel form).

Impregnated Hydrogel dressings: gauzes and non-woven sponges, ropes and strips saturated with an amorphous hydrogel.

Hydrogel sheet dressings: three-dimensional networks of cross-linked hydrophilic polymers that are insoluble in water with the capacity to interact with aqueous solutions by swelling.

Impregnated dressings: gauzes and non-woven sponges, ropes and strips staturated with a solution, an emulsion, oil or some other agent or compound. Agents most commonly used include saline, oil, zinc salts, petrolatum, xeroform and scarlet red.

Absorptive dressings: sheets and other shapes of foamed polymer solutions (most commonly polyurethane) with small, open cells capable of holding fluids.

Alginate dressings: non-woven, non-adhesive pads and ribbons composed of natural polysaccharide fibers or xerogel derived from seaweed.

Antimicrobial dressings: sponges, impregnated woven gauzes, film dressings, absorptive dressings, nylon fabric, non-adherent barriers and combination dressings that deliver antimicrobial agents such as sliver, polyhexamethylene biguanide (PHMB), iodine or antibiotics like gentamicin, tetracyclin, clindamycin, neomycin, mupirocin, polymyxin B, bacitracin and erythromycin.

Composite dressings: dressings that combine physically distinct components into a single product to provide multiple functions, such as a bacterial barrier, absorption and adhesion. Usually, they are include multiple layers and incorporate a semi- or non-adherent pad that covers the wound.

Solutions: liquid mixtures of multiprotein material and other elements found in the extracellular matrix (for example, Hycoat® containing sodium hyaluronate).

Wound fillers: beads, creams, foams, gels, ointments, pads, pastes, pillows, powders, strands or other non-adherent formulations suitable for maintaining a moist environment and managing exudate. They may include antimicrobial agents and be suitable for deep wounds that need packing

Topical wound dressings: creams, emulsions, oils and sprays to provide moisture, local anaesthetics/analgesics and anti-microbials to the wound bed or support wound debridement or odour reduction.

Surgical and wound closure devices: surgical and wound adhesives, fibrin sealants or glues, sutures, staples and strips.

As described previously herein, the present invention may also be used for skin therapy, including for example cosmetic treatment, treatment and closure of tissue openings and superficial wounds, treating damaged skin or skin conditions associated with excessive collagen breakdown, ECM loss such as Stevens-Johnson Syndrome, UV-sunlight exposure, steroid therapy, and Cushing's syndrome.

Furthermore the present invention can be used in most applications in the dermis where ECM is deficient or where ECM needs to be replaced, and where the endogenous production of ECM provides the additional benefit of the ECM proteins being locally excreted and deposited into a naturally formed matrix which provides a more durable matrix due to its normal cell-matrix, matrix-matrix interactions and cross linking, compared to non-cross linked exogenous collagen which is prone to rapid degradation by MMPs and other biological processes after implantation.

In one embodiment, the present invention may be used to prevent, treat or ameliorate a loss or lack of ECM caused by insufficient ECM production. Insufficient ECM production can be caused by an insufficient TGF-β-mediated ECM stimulation. Insufficient TGF-β-mediated ECM stimulation can be for example caused by a decrease in the amount of TGF-β, an increase in the amount of soluble TGF-β receptor, inhibition of TGF-β receptor or receptor signalling, anti-TGF-β immunotherapies, SMAD inhibitors, steroids, other therapies which negatively effect TGF-β mediated

ECM stimulation. In one example, insufficient ECM production is the result of the presence of steroids, such as for example by steroid therapy (topical or systemic). Alternatively an excess of steroids is caused by Cushing's syndrome.

Loss of ECM is also caused by excessive ECM protein breakdown, e.g. collagen breakdown.

As described previously, where appropriate the pharmaceutical compositions of the present invention are administered topically in the form of a lotion, solution, cream, ointment, or dusting powder, or use of a skin patch. Compositions of the present invention may also contain one or more additional agents such as for example lidocaine (local anaesthetic/analgesic), MMP inhibitors and anti-inflammatory agents (corticosteroids).

In this case, formulations may also further include one or more of the following: a steroidal anti-inflammatory drug (corticosteroid), a calcineurin inhibitor (eg pimecrolimus, tacrolimus), a phosphodiesterase inhibitors, an anti-histamine, an anti-microbial agent, a antibiotic, a antibacterial agent, a ceremide, a growth factor (eg transforming growth factors β1-3, platelet derived growth factor, fibroblast growth factor, insulin-like growth factors I & II, epidermal growth factor, keratinocyte growth factor, nerve growth factor), a mitogenic agent, a MMP inhibitor (eg TIMP's, Batimastat, Marimastat, and matlystatin B), a protease inhibitor, an angiogenic promoter, a chemotactic agent, inhibitor, a ECM protein, tretinoin (Vitamin A), a antioxidant (vitamins E and C), a plant cytokinin (kinerase), a copper-peptide complexes as well as numerous plant, animal and mineral extracts (ie coal tar extract).

In one embodiment, the formulations include a combination with a steroidal anti-inflammatory drug. Another composition includes a calcineurin inhibitor. Another composition includes an anti-histamine. Another composition includes an anti-microbial agent. Another composition includes a growth factor. Another composition includes a protease inhibitor. Another composition includes an angiogenic promoter. Another composition includes a chemotactic agent.

Examples of topical formulations including a agent with peroxidase activity are described below.

(a) Formulation

The topical composition of the invention may include the agent with peroxidase activity in an amount of between about 0.1% and about 75% weight/volume, generally 1%-40%, typically about 1%-10%, and usually 0.5%-5% (weight/volume).

In one embodiment, the application of the topical application of a composition to the skin of an animal or human in need thereof provides skin care benefits.

Skin care benefits achieved following topical application of the composition include for example those selected from treating/reducing wrinkling, sagging, scarring, aged and/or photo-damaged skin; boosting collagen deposition in skin, enhancing tissue repair; improving skin texture, smoothness and/or firmness, or a combination thereof. These may be considered to be cosmetic skin benefits. Naturally, the amount of benefit achieved, for example the amount of reduction in the appearance of wrinkles, sagging, and so forth will depend on the nature of the treatment, the condition of the skin prior to treatment and the length of treatment. Measurement of a skin care benefit is somewhat subjective. In one embodiment, the reduction of wrinkling, sagging, etc. of a subject's skin is a reduction compared to the condition of the skin prior to initiating treatment and is apparent to the naked eye. A range of non-invasive methods can be utilised to measure the amount of benefit achieved, for example ultrasound can be used measure skin thickness, with an increase in skin thickness representative of a beneficial effect. Silicone dental impression material can be used to take an impression of the skin before and after use of the composition and the impression analysed by a profilometer to assess the roughness of the skin surface with decreased roughness representative of a beneficial effect. A hand-held uniaxial extensometer can be used to assess the extensibility of skin with a greater resistance to skin stretching representative of a beneficial effect. Digital images can be captured of the skin being treated with the composition using a facial imaging system such as the Rapid Evaluation of Anti-aging Leads (REAL; Proctor and Gamble Co, Cincinnati, Ohio USA). The captured images can be analysed to measure the wrinkle and depression area of selected facial regions with a reduction of the area representative of a beneficial effect. Transepidermal water loss (TEWL) can also be measured using a Dermalab® TEWL instrument (Cortex Technology, Hadsund, Denmark) or similar apparatus with a decrease in TEWL representative of a beneficial effect on the skin barrier.

In other embodiments, the composition may be used for treating damage or trauma to the skin such as for example in the case of a superficial wound, burn (eg. from a hotplate) or sunburn, or from physical, chemical, environmental, disease or age related damage or as a result of a medical or pharmaceutical treatment.

(b) Other Ingredients

In one embodiment, the topical composition includes another active ingredient, such as for example, a steroidal anti-inflammatory drug (corticosteroids) suitable for topical administration.

Other additional agents include growth factors, tretinoin (Vitamin A), antioxidants (vitamins E and C), alpha-hydroxy acids (glycolic and lactic acid), plant cytokinin (kinerase), copper-peptide complexes as well as numerous plant and animal extracts.

The composition may also include a dermatologically/cosmetically acceptable vehicle to act as a diluent, dispersant or carrier for the actives. The vehicle may include materials commonly employed in skin care products such as water, liquid or solid emollients, silicone oils, emulsifiers, surfactants, solvents, humectants, thickeners, powders, propellants and the like.

The vehicle will generally form from 5% to 99.9%, typically from 25% to 90%, and usually 30% to 80% by weight of the composition, and can, in the absence of other cosmetic adjuncts, form the balance of the composition.

Besides the actives, other specific skin-benefit actives such as sunscreens, skin-protectant agent, skin-soothing agent, moisturizers, skin-lightening agents, skin tanning agents may also be included. The vehicle may also further include adjuncts such as antioxidants, perfumes, stabilizers, penetration enhancers, lubricants, anti-microbial agents, opacifiers, preservatives, colourants and buffers. Further, the composition may also include other natural or nutraceutical products.

Accordingly, in an alternate embodiment the topical composition of the present invention includes one or more ingredients selected from the group including for example, talc, glycerin, glycerol, octyl salicylate, diisopropyl adipate, glyceryl stearate, isopropyl palmitate, hyaluronic acid, oleyl alcohol, cetearyl alcohol, ethyl hexyl methoxycinnimate, stearic acid, cetearyl alcohol, dimethicone, triethanolamine, xanthan, imidazolidinyl urea, carbomer, tocopherol acetate, diazolidinyl urea, phenoxyethanol, carbomer iodopropynyl butylcarbamate, alpha lipoic acid, sodium hyaluronate, glucosamine HCl, allantoin, tocopherol acetate, green tea extract, shea butter, grape seed extract, aloe barbadensis leaf juice, cottonseed oil, avocado oil, grapefruit seed oil, allantoin and liposomes.

(c) Product Preparation, Form, Use and Packaging

To prepare the topical composition used in the method of the present invention, the usual manner for preparing skin care products may be employed although care must be taken to avoid conditions that may result in protein denaturation such as temperatures above 60° C. The active components can suitably first be dissolved or dispersed in a portion of the water or another solvent or liquid to be incorporated in the composition. Typical compositions are oil-in-water or water-in-oil or water-in-oil-in-water emulsions.

The composition may be in the form of conventional skincare products such as a cream-gel, or lotion, capsules or the like. The composition can also be in the form of a so-called “wash-off” product, e.g. a bath or shower gel, possibly containing a delivery system for the actives to promote adherence to the skin during rinsing. In one embodiment, the product is a “leave-on” product; that is, a product to be applied to the skin without a deliberate rinsing step soon after its application to the skin.

The composition may be packaged in any suitable manner such as in a jar, a bottle, tube, roll-ball, pump, spray or the like, in the conventional manner.

The composition can be applied using a material which is optionally pre-soaked in the composition of the invention. For example the material can be a bandage, gauze band-aid, sponge or the like which can be used to apply the composition of the invention for any period of time.

The present invention may be carried out one or more times daily to the skin which requires treatment. The improvement in skin appearance will usually become visible after 3 to 6 months, even after only 2-3 applications depending on skin condition, the concentration of the active components used in the inventive method, the amount of composition used and the frequency with which it is applied. In general, a small quantity of the composition, for example from 0.1 to 5 gram is applied to a suitable area of the skin from a suitable container or applicator and spread over and/or rubbed into the skin using the hands or fingers or a suitable device. A rinsing step may optionally follow depending on whether the composition is formulated as a “leave-on” or a “rinse-off” product.

It will be understood that some cosmetic and therapeutic compositions may be made available to the general public as “over the counter” or non-prescription products. In one embodiment the topical composition is suitable to be made available to the general public over the counter. In an alternative embodiment the topical composition is not suitable to be made available to the general public over the counter. This may particularly be the case in an embodiment including other active ingredients, such as for example a topical steroid or NSAID.

The present invention also provides methods of screening for agents that promote production of one or more components of extracellular matrix by a fibroblast cell in a biological and/or promote migration of fibroblast cells in a biological system.

Accordingly, in another embodiment the present invention provides a method of identifying an agent that promotes production of one or more components of extracellular matrix by a fibroblast cell in a biological system and/or promotes migration of a fibroblast cell in a biological system, the method including:
(i) providing an agent with peroxidase activity; and
(ii) determining the ability of the agent to promote the production of one or more components of extracellular matrix by a fibroblast cell in a biological system and/or the ability of the agent to promote migration of a fibroblast cell in a biological system; and
(iii) identifying an agent that promotes production of one or more components of extracellular matrix by a fibroblast cell and/or promotes migration of a fibroblast cell in a biological system.

The present invention also provides agents identified according to the above method.

Methods for determining the ability of fibroblast cells to promote production of one or more components of extracellular matrix are known in the art. Similarly, methods for determining the ability of the agent to promote migration of a fibroblast cell in a biological system are also known in the art.

The above screening methods may also be used to identify agents that promote a fibrogenic response.

Finally, standard techniques may be used for recombinant DNA technology, oligonucleotide synthesis, and tissue culture and transfection (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification, which are hereby incorporated by reference. See e.g., Sambrook et al. Molecular Cloning: A Laboratory Manual (2d ed., Cold Spring Harbor Laboratory Press, Cold Spring Harbor, N.Y. (1989) and Ausubel, F. M. et al. (1989) Current Protocols in Molecular Biology, John Wiley & Sons, New York, N.Y.; Animal Cell Culture: Practical Approach, Third Edition (John R. W. Masters, ed., 2000), ISBN 0199637970, whole of text; Immobilized Cells and Enzymes: A Practical Approach (1986) IRL Press, Oxford, whole of text; and Immunology, Fifth Edition (Goldsby R. A, Kindt T. J, Osborne B. A and Kuby J, 2003), whole of text.

### Description of Specific Embodiments

Reference will now be made to experiments that embody the above general principles of the present invention. However, it is to be understood that the following description is not to limit the generality of the above description.

### Example 1

Proteins with Peroxidase Activity Stimulate Production of an Array of ECM Proteins by Human Fibroblast Cells

Primary human foreskin fibroblast cells (HFF) were seeded into 96 well plates at a density of 1.2×10⁴ cells/well and cultured overnight in 10% fetal bovine serum/Dulbecco's minimum essential media (10% FBS/DMEM) at 37° C. and 5% CO₂. The cells were then starved overnight in serum free DMEM and triplicate wells treated for six hours with a range of doses of proteins with peroxidase activity; horse radish peroxidase (HRP; 0.2-50 ug/ml), Arthromyces ramosus peroxidase (ARP; 0.2-50 ug/ml), microperoxidase (MP; 8-500 ug/ml), soy bean peroxidase (SBP; 0.2-100 ug/ml) and lactoperoxidase (LP; 10-310 ug/ml) (all obtained from Sigma-Aldrich). At the end of the six hour treatment period, the media was aspirated and replaced with fresh serum free DMEM and the cells cultured for a further 72 hours. After 72 hours the culture media was collected for assessment of secreted, soluble collagen types I, III and VI as well as fibronectin protein and the cells assessed for viability/growth using an alamar blue fluorescent dye assay. This assay measures the reduction of oxidized, blue nonfluorescent Alamar Blue reagent to a pink fluorescent dye in the cell medium, such that the higher the amount of reduction, the greater the cell number and/or activity (O'Brien Eur J Biochem 267 2000). Briefly, cells were bathed in a 10% Alamar Blue/phosphate-buffered saline (PBS) solution for 30-60 minutes and fluorescence measured at wavelengths of 485 nm excitation and 595 nm emission using a Wallac Victor Multilabel HTS counter. Relative fluorescent units (RFU) for each sample were normalized to control wells (DMEM only treated cells) and data expressed as mean±standard error of the mean (sem) for triplicate wells. Experiments were repeated at least three times with the combined data from all experiments shown.

The amount of soluble collagen types I, III, and VI in HFF conditioned media was measured by a direct coat ELISA method using standard curves constructed from purified human collagen I, III or VI (extracted from human placenta; col I, BD Biosciences; col III and VI, Rocklands Immunochemicals) and fibronectin (extracted from human plasma, Chemicon). Samples and standards (20 μl/well) were added to a 384 well spectraplate (Packard BioScience) and left at room temperature (RT) overnight. The spectraplate was then washed 6 times with PBS-tween 0.05% (PBS-T) and 2.5% bovine serum albumin (BSA)/PBS blocking solution added to each well and the plate incubated for 1 hour at RT. The plate was then washed 6 times with PBS-T and primary antibody (0.25 μg/ml rabbit-anti-human-collagen I polyclonal; 2 μg/ml rabbit-anti-human-collagen III polyclonal; 0.25 μg/ml rabbit-anti-human-collagen VI polyclonal; 0.2 μg/ml rabbit-anti-human-fibronectin polyclonal, all from Rockland Immunochemicals) in 5% non-fat dairy milk added to each well for four hours at RT. After washing (6×PBS-T), europium tagged anti-rabbit secondary antibody (0.5 μg/ml 1% BSA/PBS; Wallac Oy) was added for 1 hour at RT. After a final wash (6×PBS-T), enhancement solution was added for at least 10 minutes and fluorescence measured at excitation 340 nm and emission 615 nm using a Wallac Victor Multilabel HTS counter. The respective ECM concentration of each sample was determined from the standard curve (μg/ml) and normalized to control wells (DMEM only treated cells) with the mean±sem for triplicate wells calculated. Experiments were repeated at least three times with the combined data from all experiments shown.

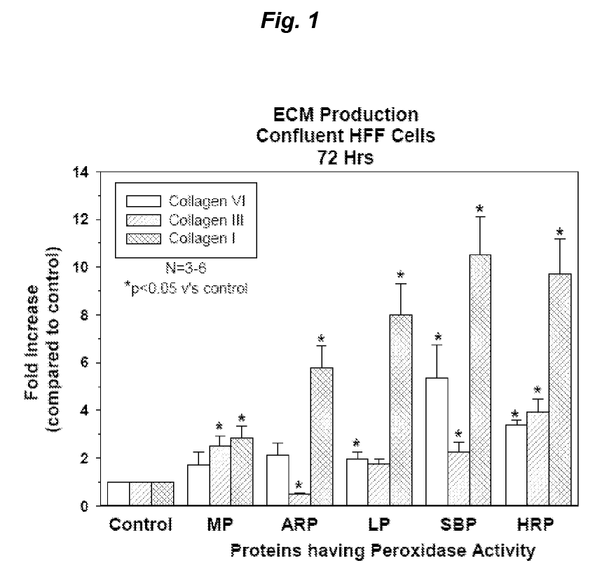

It was found that a 6 hour treatment with MP, SBP, ARP, HRP or LP surprisingly increased ECM protein levels dose dependently in the media of HFF cells over the following 72 hours. FIG. 1 shows representative increases in collagen I, III and VI levels stimulated by each of the proteins with peroxidase activity after treating the cells with concentrations ranging between; 8-500 μg/ml MP, 0.4-50 μg/ml SBP, 0.2-50 μg/ml ARP, 0.2-50 μg/ml HRP and 10-310 μg/ml LP.

Collagen I results: LP induced a significant increase (p<0.05) in collagen I at doses between 80 and 310 μg/ml, ranging from a 4-fold increase at 80 μg/ml to a 8-fold increase compared to controls at 310 μg/ml (shown in FIG. 1). HRP induced a significant increase in collagen I at each dose tested, ranging from a 1.5-fold increase at 0.2 μg/ml to a 10-fold increase compared to controls at 50 μg/ml (shown in FIG. 1). A steady state response of between 9-10 fold was evident for doses between 6.25 and 50 μg/ml. ARP induced a significant increase in collagen I from 0.4 μg/ml (1.4-fold), reaching a 5.8-fold increase compared to controls at 25 μg/ml (shown in FIG. 1) with a steady state response between 5.4 and 5.8-fold observed for doses between 6.25 and 50 μg/ml. SBP induced a significant increase in collagen I at each dose tested, ranging from a 1.6-fold increase at 0.4 μg/ml to a 10.5-fold increase at 25 and 50 μg/ml (shown in FIG. 1). MP induced a significant increase in collagen I of between 2.5 and 3-fold compared to controls at concentrations of 250 and 500 μg/ml (250 μg/ml response shown in FIG. 1).

Collagen III results: LP induced only small, non-significant 1.5-1.7-fold changes in collagen III levels at doses from 10-310 μg/ml (40 μg/ml result shown in FIG. 1). HRP induced a significant increase in collagen III ranging from a 3-fold increase at 3.12 μg/ml to a 4-fold increase compared to controls at 25 and 50 μg/ml (shown in FIG. 1). ARP did not induce a significant increase in collagen III at any dose used and in fact, caused a gradual, dose-dependent decrease compared to control levels which reached a significant 50% decrease at the 25 and 50 μg/ml concentrations (25 μg/ml result shown in FIG. 1). SBP induced a significant increase in collagen III that reached a steady state 2.1-2.3-fold increase between the 12.5 and 50 μg/ml concentrations (50 μg/ml result shown in FIG. 1). MP induced a significant 2 to 2.5-fold steady state increase in collagen III at doses between 62 and 500 μg/ml (500 μg/ml response shown in FIG. 1).

Collagen VI results: LP induced a significant 2-fold increase in collagen VI levels at the 160 and 310 μg/ml concentrations (shown in FIG. 1). HRP induced a significant increase in collagen VI ranging from a 2-fold increase at 1.56 μg/ml to a steady state 3-3.5 fold increase compared to controls at doses between 6.25 and 50 μg/ml (25 μg/ml response shown in FIG. 1). ARP induced a steady state 2-fold increase in collagen VI at doses ranging from 6.25 μg/ml to 50 μg/ml although overall, the response was not statistically significant (p=0.08, 25 μg/ml response shown in FIG. 1). SBP induced a significant increase in collagen VI at all doses tested and ranged from 1.2-fold at 0.4 μg/ml to 5.4-fold at 50 μg/ml (shown in FIG. 1). MP did not induce a significant increase in collagen VI at any dose used (500 μg/ml response shown in FIG. 1).

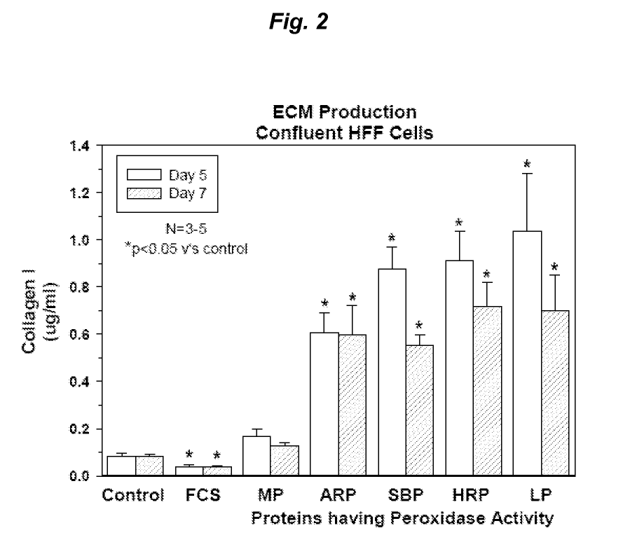

To ensure the production of ECM proteins stimulated by a six hour exposure of confluent foreskin fibroblast cells to proteins having peroxidase activity lasted more than 3 days, additional experiments were performed using representative doses of each protein having peroxidase activity (ie ARP, HRP and SBP; 12.5 μg/ml; LP 160 μg/ml; and MP 250 μg/ml) and the levels of ECM proteins in media collected from the cells after 5 or 7 days measured. Compared to the collagen I concentration of 0.08 μg/ml in the media of untreated control cells, all proteins having peroxidase activity except MP significantly elevated (p<0.05) collagen I levels over 5 days (FIG. 2). The collagen I concentration ranged from 0.17 μg/ml for cells treated with MP to 1.1 μg/ml for cells treated with LP and actually decreased significantly to 0.04 μg/ml for cells treated with 10% FBS (FIG. 2). Collagen I levels in the media of cells treated with each of the proteins having peroxidase activity other than MP were also significantly greater than untreated controls after 7 days (FIG. 2). Whilst the collagen I concentration in the media of the day 7 control cells and cells treated with ARP was the same as the day 5 cells (0.08 μg/ml and 0.6 μg/ml respectively), the concentration in the day 7 cells treated with SBP, HRP and LP was some 20-30% lower than the day 5 cells. This indicates that either the rate of collagen I production slowed in these cells (relative to the rate of incorporation into the extracellular matrix) or that more soluble collagen I was processed and incorporated into the cell-associated ECM.

Small increases in collagen III levels were evident after 5 days in cells treated with SBP (0.15 μg/ml) and LP (0.18 μg/ml) compared to untreated control cells (0.11 μg/ml) with only HRP inducing a significant change (0.22 μg/ml). The level of collagen III after 7 days was found to be generally similar to that measured after 5 days and reached a maximum of 0.3 μg/ml in cells treated with HRP (12.5 μg/ml) which was significantly greater (p<0.05) than the 0.06 μg/ml secreted by untreated control cells. The outcome was much the same when collagen VI was measured in the cell media as LP, SBP, ARP and HRP all increased the collagen VI levels at both 5 and 7 days. The 7 day responses (LP; 0.07 μg/ml, SBP; 0.09 μg/ml, ARP; 0.11 μg/ml, HRP; 0.13 μg/ml) were all significantly greater than the untreated control levels of 0.03 ug/ml.

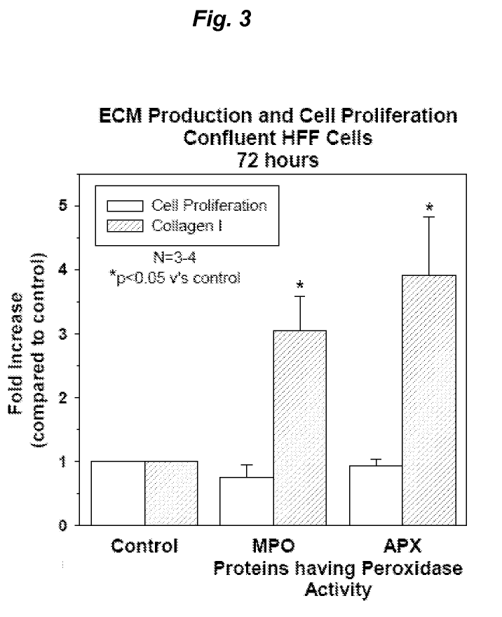

Additional experiments were also performed to determine the ability of myeloperoxidase (MPO; 30 μg/ml) and recombinant ascorbate peroxidase (APX; 3-700 μg/ml) to stimulate ECM production by HFF cells after 72 hours. A dose-dependent increase in collagen I production was induced by APX and ranged from 1.4-fold at a 5 μg/ml dose to a significant 3.9-fold increase at 175 μg/ml (FIG. 3). Steady state levels ranged between a 3 and 4-fold increase at doses between 80 and 700 μg/ml of APX. MPO induced a significant 3-fold increase at the 30 μg/ml dose used (FIG. 3). Neither APX nor MPO had a major effect on HFF cell growth or viability (FIG. 3), with this being a consistent finding across all proteins with peroxidise activity tested.

To ensure the soluble ECM proteins stimulated by the proteins with peroxidase activity are incorporated into the extracellular matrix generated by human fibroblast cells, given that this is a key requirement for the regeneration of collagen-rich tissues like skin and other organs and tissues, total proteins were extracted from the cell matrix. In these experiments, six well culture plates were seeded with fibroblast cells isolated from an adult subject at a density of 1.5×10⁵ cells/well and after one week of culture, cells were treated with proteins with peroxidase activity in media containing 10% FBS and with the addition of ascorbic acid (vitamin C) which is a well known cofactor important for the cross linking of collagen molecules to generate polymeric fibres. Wells were supplemented daily to provide a final concentration of 50 ug/ml ascorbic acid in the media. After 72 hours the cell media was removed, the wells washed with warm PBS and the cell matrix extracted.

The total protein content of the cell matrix was solubilised by the addition of 500 ul/well of RIPA buffer (25 mM Tris-HCl pH7.4; 150 mM NaCl; 1% Triton X-100; 0.5% Na deoxycholate; 0.05% SDS). The cell matrix was scraped off the bottom of each well and the plates placed on a shaking platform for 10 minutes. The extracts were transferred to eppendorf tubes, vortexed vigorously and centrifuged at 12,000 rpm, 4° C. for 5 minutes. The DNA pellet was visualised and gently removed and the samples stored at −80° C. until assessment.

Total cell extracts were assessed by polyacrylamide gel electrophoresis. Samples were prepared by adding an appropriate amount of reduced loading buffer to neat cell extracts and boiling for 5 minutes. Samples were then loaded onto a 4-12% BisTris gradient gel (Invitrogen) together with molecular weight markers (Magic Mark XP and SeeBlue prestained markers; Invitrogen) and resolved for 5 hours at 150V in a Novex mini-cell system (Invitrogen) using NuPage MES SDS running buffer. After resolution, the gel was rinsed in water and fixed for 30 minutes in 50% methanol/5% acetic acid and washed again in water for 30 minutes. Gels were then used to visualise resolved proteins by silver staining Silver staining was performed by sensitizing the gel for 1 minute in 0.02% sodium thiosulphate and after rinsing in water, incubating the gel in 0.1% silver solution for 20 minutes. After rinsing in water, the gel was developed for 5-10 minutes in 2% NaOH/0.014% formaldehyde solution with the development stopped by placing the gel in a 1% acetic acid solution.

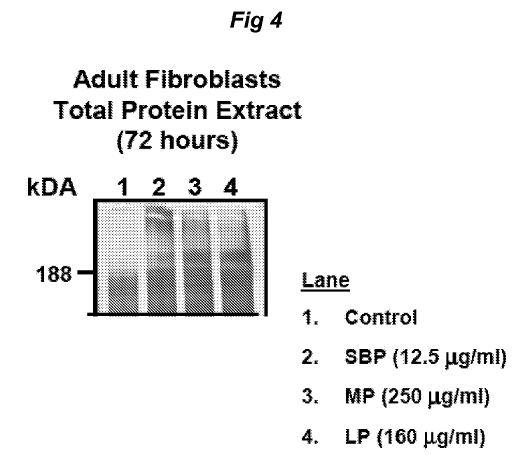

FIG. 4 demonstrates that adult fibroblast cells cultured under fully supplemented conditions (10% FBS+50 ug/ml ascorbic acid) and treated with SBP, MP and LP for 72 hours produced much more proteinaceous material at a range of sizes than the untreated control cells (cultured with 10% FBS and ascorbic acid alone). Specifically, FIG. 4 shows that cells treated with proteins having peroxidase activity deposit within the extra-cellular matrix greater quantities of high molecular weight proteins, particularly in the size range expected for proteins like fibronectin (220 kDa).

### Example 2

ECM Protein Production by Human Foreskin Fibroblast Cells can be Stimulated Repeatedly by Proteins with Peroxidase Activity

Experiments were performed to ensure human fibroblast cells could be stimulated to increase ECM protein production by proteins having peroxidase activity after more than one application of the proteins having peroxidase activity. The capacity to respond repeatedly to the proteins having peroxidase activity provides important utility for the generation of ECM in vitro, particularly given that spent culture media needs to be replaced on a regular basis to maintain the viability of the cells. These experiments were performed using human foreskin fibroblast cells (HFF cells) seeded into 96 well plates at a density of 1.2×10⁴ cells/well and cultured for one week in 10% fetal bovine serum/Dulbecco's minimum essential media (10% FBS/DMEM) at 37° C. and 5% CO₂ (as described in Example 3). The confluent cells were then starved overnight in serum free DMEM and triplicate wells treated for six hours with MP, ARP and LP as representative proteins having peroxidase activity or the control agents transforming growth factor β2 (TGFβ2; R&D Systems) and 10% FBS (JRH BioSciences). After 48 hours, the conditioned media was collected for measurement of collagen I (as a representative ECM protein) and the cells either restimulated for six hours with fresh solutions containing proteins having peroxidase activity or control agents or the media was replaced with fresh serum free DMEM. The cells were cultured for a further 5 days after which the media was collected a second time for the measurement of collagen I. Media was also collected from replicate wells treated at the start of the experiment and left for 7 days (total culture period) for comparison.

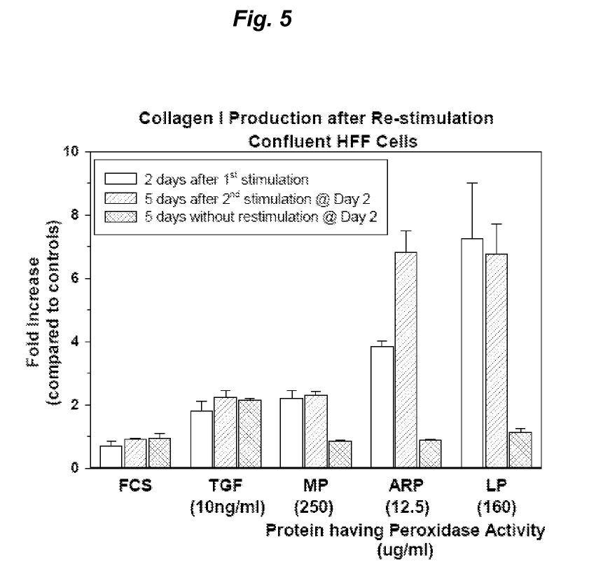

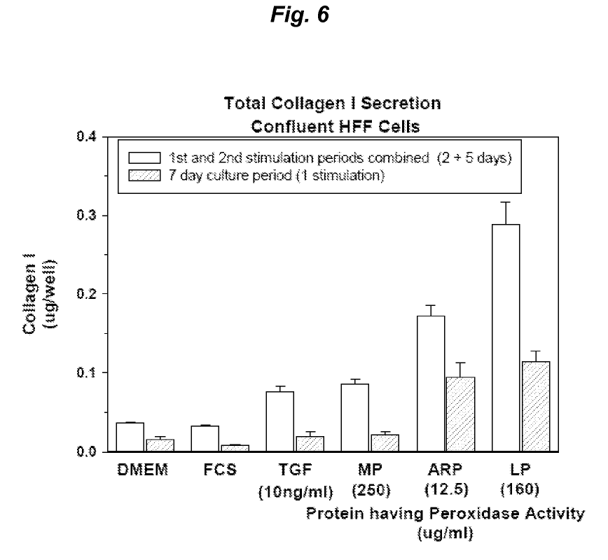

FIG. 5 shows that relative to time-matched, untreated controls, a second stimulation with MP and LP stimulated an increase in collagen I production that was at least equivalent to that obtained with the first stimulation. Cells treated with ARP surprisingly produced a greater response when stimulated a second time (FIG. 5). It was also noted that all of the cells initially treated with proteins having peroxidase activity, but left untreated after the change of media after day 2, only produced baseline levels of collagen I. This was different to cells treated initially with 10% FBS or TGFβ2 which showed a maintained level of collagen I production when left untreated after the media change at day 2, although the degree of overall stimulation was much less than that observed for ARP and LP (FIG. 5). When the total amount of collagen secreted per well was calculated for each stimulation period and summed, it was found that the proteins having peroxidase activity stimulated the production of more collagen I when the cells were stimulated twice within a seven day culture period compared to one stimulation at the start of the experiment (FIG. 6). The response of cells treated with 10% FBS was no different to the untreated control cells whilst cells treated with MP responded in a similar way to cells treated with TGFβ2. Overall, cells treated with ARP and LP produced markedly greater total collagen I levels when they were re-stimulated during the culture period (FIG. 6).

### Example 3

Proteins having Peroxidase Activity Promote the Infiltration of Human Fibroblast Cells into Tissue Regeneration Scaffolds

Human dermal fibroblast cells, both autologous and heterologous, can be seeded onto a variety of three-dimensional frameworks or scaffolds made from natural ECM based proteins or biocompatible synthetic polymers, or suspended in a range of semi-solid matrices such as collagen or protein hydrogel matrices using conventional technology. For example, cells can be seeded onto a porous matrix of fibers of cross-linked bovine tendon collagen and a glycosaminoglycan such as chondroitin-6-sulphate. One such example of a dermal regeneration scaffold composed of collagen and glycosaminoglycan is INTEGRA®. Experiments were undertake to determine the effect of proteins having peroxidase activity on the ability of fibroblast cells (primary adult-derived fibroblast cells and HFF cells) to populate three-dimensional frameworks or scaffolds and semi-solid matrices and create a three-dimensional human tissue by producing collagen and other ECM components to fill the interstices of the framework or scaffold.

INTEGRA® was cut into lcm x lcm pieces using a sterile scalpel, the pieces transferred to a petri dish using sterile forceps and washed four times each with PBS followed by serum-free DMEM, to remove the alcohol storage medium and to equilibrate the porous scaffold for exposure to cells. After the last wash, the INTEGRA® pieces were transferred to the wells of a 12 well tissue culture plate (collagen layer facing up) immersed in media containing proteins having peroxidase activity. This “pre-treatment” of the INTEGRA® with proteins having peroxidase activity was performed for up to 16 hours at 37° C. in a CO₂ incubator. After the “pre-incubation” period, the media was replaced with fresh DMEM supplemented with 10% FBS (ie normal growth media as the control) containing 5×10⁵ human fibroblast cells plus or minus proteins having peroxidase activity. After allowing 24 hours for the fibroblast cells to attach to the collagen matrix of the INTEGRA® scaffold, the INTEGRA® pieces were transferred to fresh 12 well plates to ensure any cells not attached to the matrix surface were removed. Fresh media containing proteins with peroxidase activity were added to the wells and the INTEGRA® pieces incubated for up to 14 days (“post-treatment” period) with the media changed every 7 days as necessary. At the end of the experiment the INTEGRA® pieces were harvested and fixed in 10% buffered formaldehyde before they were processed, embedded in paraffin wax, cut in cross-section and placed onto microscope slides for histological and immunohistochemical analysis.

Six to eight micron thick sections of the INTEGRA® were mounted on silane-coated slides and were dewaxed before placing into an endogenous peroxidase blocking solution for 30 min (0.5% H₂O₂/methanol). After washing in PBS, sections were incubated in 3% normal horse serum blocking solution for 30 min and then 5 μg/ml rabbit anti smooth muscle actin (SMA; AbCam, Cambridge Mass.) primary antibody overnight. The following day sections were washed in PBS and incubated with 6 μg/ml anti rabbit IgG biotin. After a further PBS wash, 2 μg/ml streptavidin HRP tertiary antibody was applied for 1 hr followed by the addition of 3,3′-diamino benzidine peroxidase substrate solution (DAB—Sigma Chemicals) to the sections for 7 min or until brown colour development was visible. The sections were then washed thoroughly in PBS and water before counterstaining with haematoxylin, dehydrating and mounting with DPX. Smooth muscle actin was used as a stain to specifically identify human fibroblast cells within the INTEGRA® scaffold.

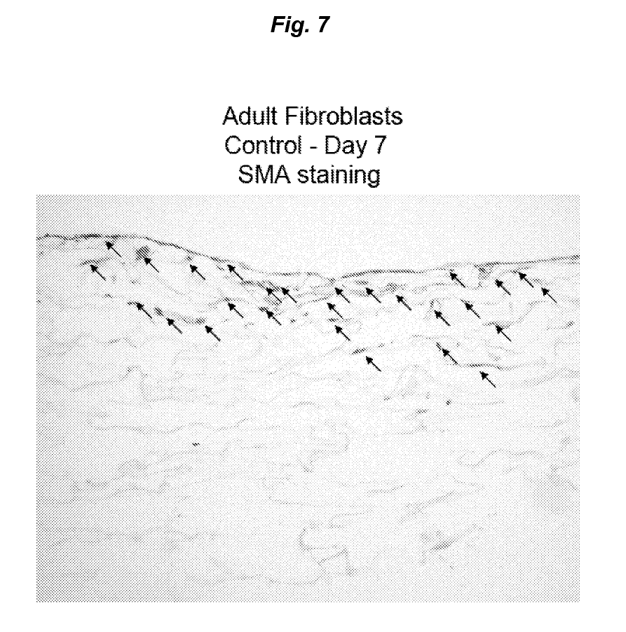

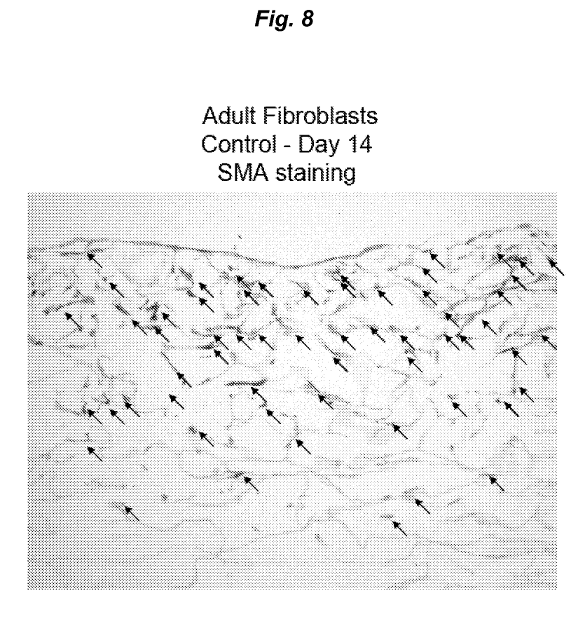

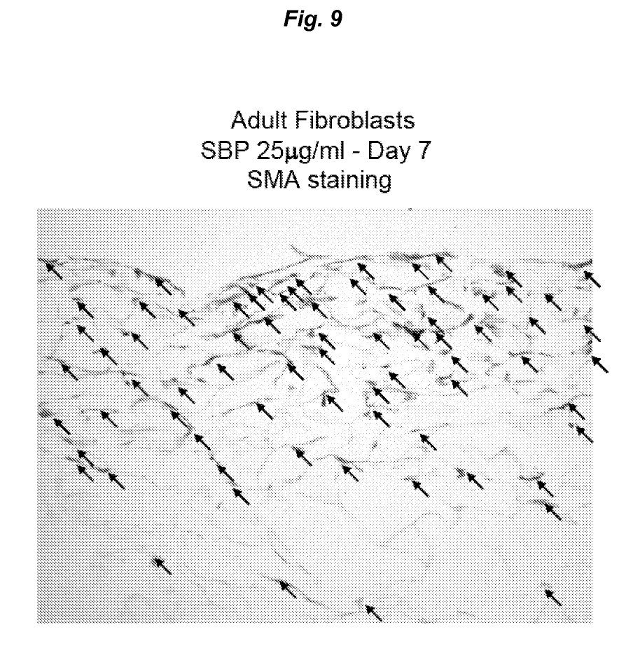

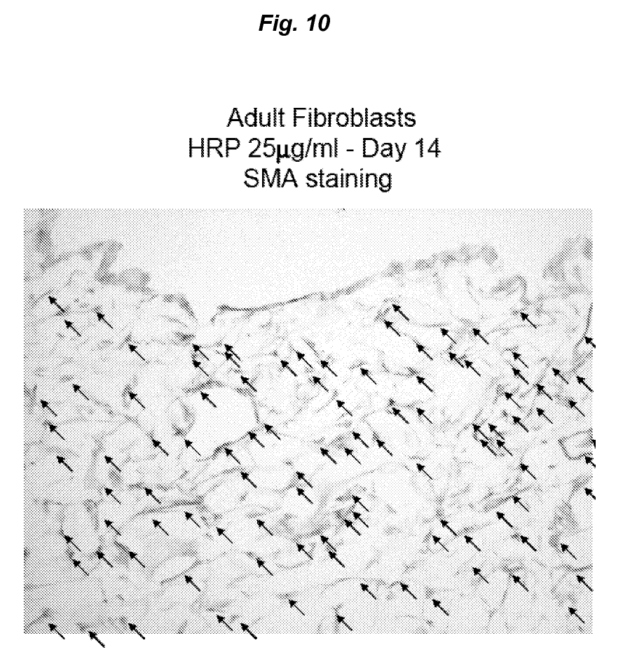

After seven days of culture in control media, only a small number of primary-adult fibroblast cells seeded onto the INTEGRA® had migrated into the scaffold as demonstrated by the SMA positive staining (highlighted by the arrows) in FIG. 7. Considerably more cells had populated the scaffold after 14 days and some cells had migrated at least ⅔rds of the way through the total thickness of the scaffold (FIG. 8). Most surprisingly however, when the INTEGRA® was incubated in media containing SBP (25 μg/ml) both before and after the addition of the fibroblast cells, many more cells were evident within the interstices of the scaffold after 7 days (shown in FIG. 9) than in both the 7 and 14 day controls. Even more surprising was the even greater number of cells evident within the scaffold after 14 days of treatment with HRP (25 μg/ml) and shown in FIG. 10. FIG. 10 shows that after 14 days, proteins having peroxidase activity have the ability to promote the migration of the fibroblast cells into and through the INTEGRA® scaffold such that much of the scaffold becomes populated with fibroblast cells. Comparing FIGS. 7 and 8 with FIGS. 9 and 10 shows proteins having peroxidase activity confer at least a 7 day advantage over standard culture techniques used for populating scaffolds and creating a three-dimensional tissue.

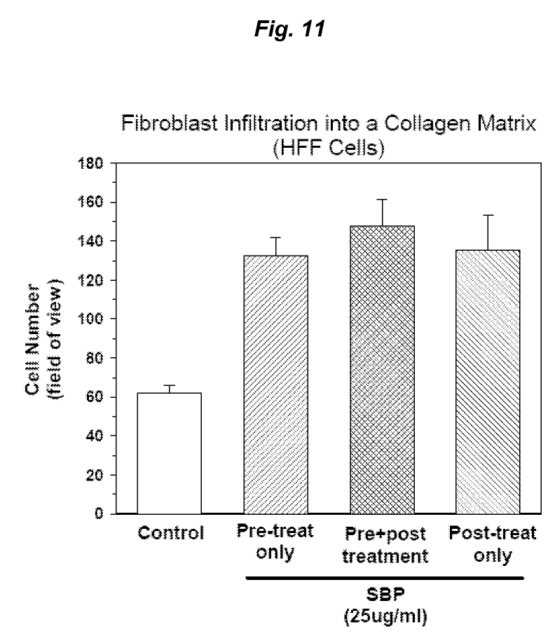

Additional experiments were performed as described above using HFF cells and varying the period of application of the proteins having peroxidase activity to include a “pre-treatment” application only (ie INTEGRA® incubated with SBP 25 μg/ml for up to 16 hours prior to the addition of cells), a “pre-treatment” and “post-treatment” application (ie INTEGRA® pre-treated with SBP and SBP added to the culture media once the INTEGRA had been seeded with cells) and a “post-treatment” application only (ie SBP added to the culture media once the INTEGRA® had been seeded with cells). The INTEGRA® was harvested after 6 days in culture and the number of SMA-positive fibroblast cells counted in five representative fields of view of sections from each condition. FIG. 11 shows surprisingly, that SBP had a major influence on the number of fibroblast cells infiltrating and populating the INTEGRA® compared to control conditions (10% FBS supplemented media). Most surprisingly, each period of application of the protein with peroxidase activity appeared as effective at promoting the infiltration and population of the INTEGRA® by HFF cells which were observed to have penetrated the full thickness of the scaffold when treated with SBP compared to controls where the penetration was limited to about the top third of the scaffold.

### Example 4

Proteins having Peroxidase Activity Promote the Production of ECM within Tissue Regeneration Scaffolds

To confirm proteins having peroxidase activity promote the generation of a three-dimensional human tissue by stimulating the production of collagen and other ECM components to fill the interstices of a three-dimensional framework or scaffold, INTEGRA® pieces collected from the experiments described in Example 3 were analysed for the production of Collagen I (as a representative ECM protein) using immunohistochemistry. Staining for collagen I was performed on sections of INTEGRA® ³ mounted on silane-coated slides following the method outlined in Example 3 for the SMA immunohistochemistry with the variation that 5 μg/ml rabbit anti Collagen-I (Rocklands Immunochemicals) was used as the primary antibody.

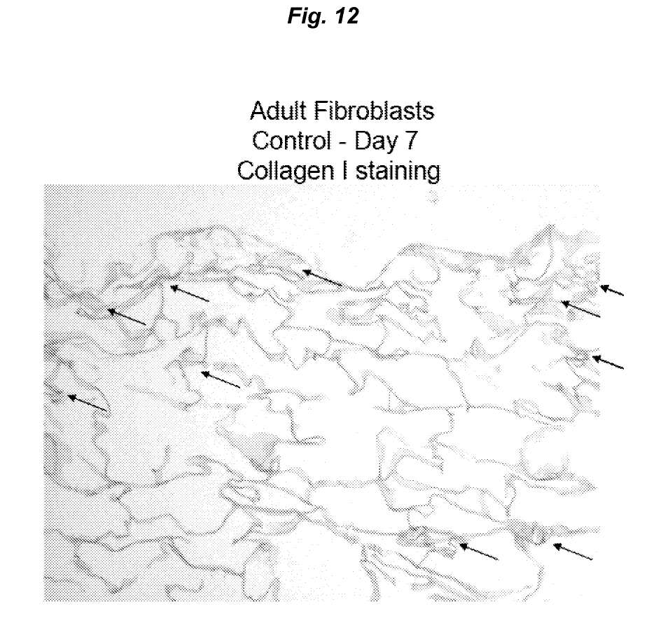

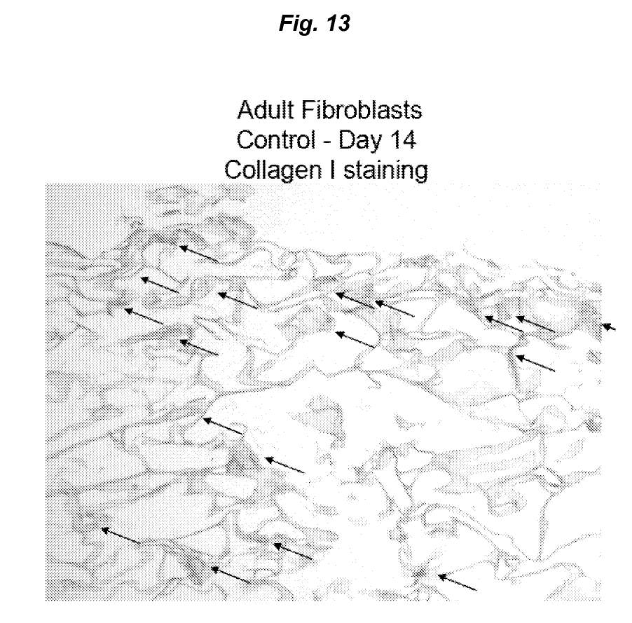

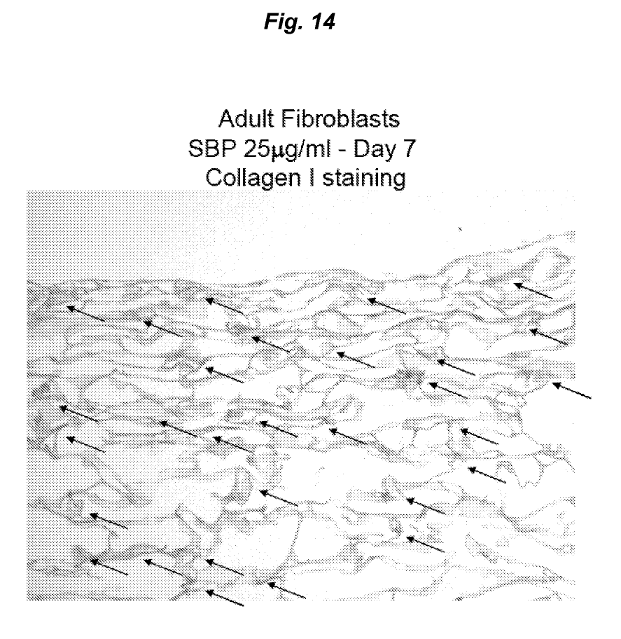

FIG. 12 shows that after seven days of culture in control media, all of the collagen I specific staining was localised to the ribbons of cross-linked bovine tendon collagen forming the structural scaffold of the INTEGRA® and there was minimal evidence of the production of new cell-associated ECM. New cell-associated ECM production was evident after two weeks of culture in control media, although this was still reasonably sparse and localised to areas where pockets of the adult fibroblast cells populated the scaffold (FIG. 13). In contrast and most surprisingly however, when the INTEGRA® was incubated in media containing SBP (25 μg/ml) both before and after the addition of the fibroblast cells, much more cell-associated ECM was evident within the interstices of the scaffold after 7 days (shown in FIG. 14) than in both the 7 and 14 day controls.

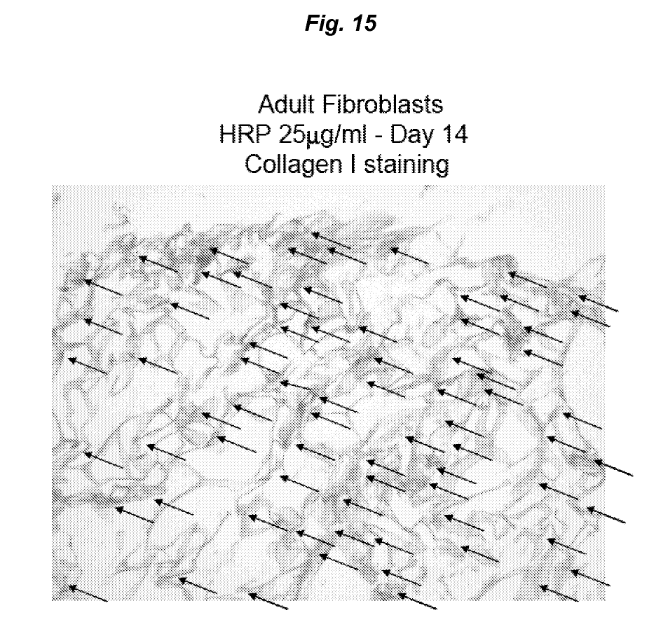

It is evident from the results shown in FIG. 14 that proteins with peroxidase activity stimulate the profuse production of collagen I that attaches to and integrates with the bovine tendon collagen forming the structural scaffold of the INTEGRA®. Given that the degree of ECM production and integration after 7 days treatment with SBP was far greater than seen after 14 days culture under standard conditions, proteins with peroxidase activity would appear to decrease the time required to fill the interstices of a three-dimensional framework or scaffold with ECM to generate a three-dimensional human tissue by at least 7-14 days compared to standard conditions. This is confirmed by the results shown in FIG. 15 where incubation with HRP (25 μg/ml) for 14 days leads to much greater ECM production and integration than observed after 14 days culture under standard conditions without the addition of proteins having peroxidase activity.

Experiments performed as described in Example 3 using HFF cells and varying the period of application of the proteins having peroxidase activity also demonstrate clearly that proteins having peroxidase activity stimulated the production and deposition of greater amounts of collagen I rich ECM within the interstices of the scaffold following either a “pre-treatment” application only (ie INTEGRA® incubated with SBP 25 μg/ml for up to 16 hours prior to the addition of cells), a “pre-treatment” and “post-treatment” application (ie INTEGRA® pre-treated with SBP and SBP added to the culture media once the INTEGRA® had been seeded with cells) and a “post-treatment” application only (ie SBP added to the culture media once the INTEGRA® had been seeded with cells). Most surprisingly, each period of application of the protein with peroxidase activity appeared as effective at promoting the production and depositon of cell-associated ECM throughout the full thickness of the scaffold when treated with SBP compared to controls where the generation of ECM within the scaffold was minimal.

Additional experiments were performed to determine if fibroblast cells populating a three-dimensional framework or scaffold could be stimulated to secrete soluble collagen I into the media bathing the three-dimensional framework or scaffold. For these experiments, HFF cells were seeded onto the INTEGRA® and allowed to populate the scaffold for two weeks under normal culture conditions (10% FCS supplementation). The populated scaffold was then stimulated with a protein having peroxidase activity by immersing it in basal media (DMEM) containing SBP (12.5 μg/ml) for 24 hours. The INTEGRA® was then placed into fresh DMEM and the collagen-I content of the media measured by ELISA after 72 hours.

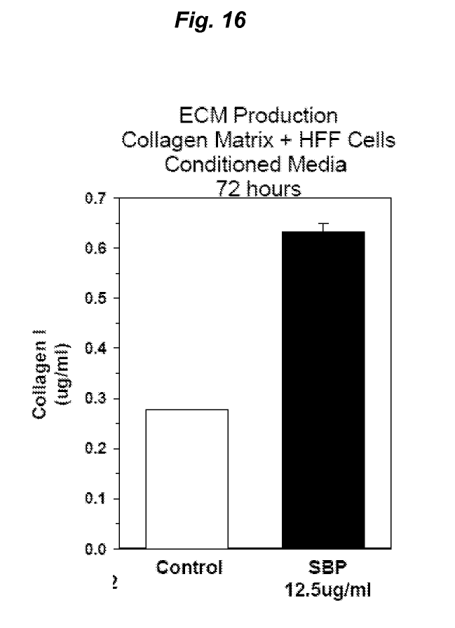

FIG. 16 surprisingly shows that applying SBP to a fibroblast-populated scaffold resulted in a 2-3 fold increase in the amount of collagen I secreted into the media bathing the scaffold. This result confirms that proteins having peroxidase activity can penetrate porous matrices and directly stimulate cells in situ to increase their production of ECM. Even more surprisingly, it would appear from this result that although a certain amount of the proteins produced by the cells are converted into insoluble, cell-associated ECM (as demonstrated in FIGS. 12-15), another certain amount of the proteins remain soluble and can be harvested from the media. This finding has great utility for increasing the generation and recovery of soluble forms of ECM that can be collected as a byproduct of the manufacture of human replacement tissues (such as skin equivalents for example) with the collected ECM used in cosmetic compositions and as intradermal tissue augmentation additives and the like.

Additional experiments were also performed to ensure the effects observed using the INTEGRA® as a three-dimensional framework or scaffold were not limited to a bovine collagen/glycosaminoglycan structure and could be achieved in synthetically derived matrices. For these experiments a PuraMatrix™ Peptide Hydrogel was used. The PuraMatrix Peptide Hydrogel is a synthetic matrix that self assembles into a three-dimensional hydrogel (1% w/v amino acids: 99% water) under physiological conditions with a nanometer scale fibrous structure and an average pore size of 50-200 nm. For these experiments, HFF cells were suspended in a 20% sucrose solution, with or without proteins having peroxidase activity, and added 1:1 to neat (1%) PuraMatrix hydrogel and carefully pipette mixed, so as not to create bubbles. Each cell/gel mix was plated in 24 well plates and allowed to partially set at room temperature for 5 min. Next, DMEM supplemented with either 10, 5 or 2% FCS was very slowly overlayed in a dropwise fashion down the edge of the well so as not to disrupt the gel. Plates were placed in a 37° C. incubator for the mix to completely solidify, and the media was changed twice over 1 hr to equilibrate the gel to pH 7.4. The encapsulated hydrogel was incubated for one week, the bathing culture media removed and RIPA buffer added to the gel for total protein extraction. The gel was aspirated back to liquid phase, placed into tubes, vortexed and then centrifuged at 12,000 rpm and 4° C. for 5 min. The supernatant was discarded and the pellet kept for protein analysis using standard BCA and western blot analysis for collagen I following polyacrylamide gel electrophoresis.

Samples were prepared for gel electrophoresis by adding an appropriate amount of reduced loading buffer to solublize the protein and boiling for 5 minutes. Samples were then loaded onto a 4-12% BisTris gradient gel (Invitrogen) together with molecular weight markers (Magic Mark XP and SeeBlue prestained markers; Invitrogen) and resolved for 5 hours at 150V in a Novex mini-cell system (Invitrogen) using NuPage MES SDS running buffer. After resolution, the gel was rinsed in water and fixed for 30 minutes in 50% methanol/5% acetic acid and washed again in water for 30 minutes.

For western blot analysis, proteins were transferred to Highbond-C nitrocellulose membrane (Amersham Biosciences) using a Novex mini-cell system and NuPage transfer buffer (Invitrogen) for 2 hours at 30V. The membrane was placed into blocking solution (5% NFDM/TBST; non-fat dairy milk/tris buffered saline-tween 0.05%) overnight at 4° C. The membrane was then incubated with the primary antibody (rabbit anti-human collagen I; Rocklands Immunochemicals) for at least 1 hour at R/T. The membrane was then washed stringently in TBST and incubated with secondary antibody (anti-rabbit HRP in 5% NFDM) for at least one hour at R/T. After washing, the membrane was developed using ECL reagents (Amersham Bioscience) as per the manufacturers instructions and an automatic photographic processor.

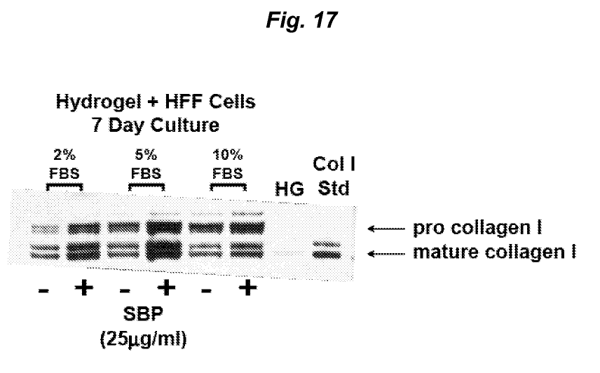

FIG. 17 gives an example of a membrane probed with anti-human collagen I antibody.

The western blot for collagen I identifies two protein bands of about 120kDa consistent with mature collagen I subunits (α1; COL1A1 and α2; COL1A2) as well as higher molecular weight species corresponding to pro-collagen I. FIG. 17 surprisingly shows that when treated with proteins having peroxidase activity, fibroblast cells encapsulated within a synthetic scaffold such as the PuraMatrix Hydrogel produce significantly more collagen I than untreated control cells. Most surprisingly, it was found that whilst cells incubated with SBP (25 μg/ml) in basal media containing 10% FCS produced more collagen I than cells incubated with 10% FCS alone, relatively more collagen I was produced when cells were incubated with SBP in 2 or 5% FCS, with 5% FCS appearing the optimal level of supplementation required to maximise the production of collagen I within the Hydrogel matrix.

### Example 5

Organisation of Collagen Fibers Formed by Human Adult Cells when Treated with Proteins having Peroxidase Activity

To ensure the proteins having peroxidase activity stimulate the production of organized

ECM, rather than disorganized ECM which would increase the risk of scar formation within the dermis and contribute to the generation of structurally and functionally impaired replacement tissue, additional experiments were performed to visualize the collagen fibers formed by fibroblast cells stimulated with proteins having peroxidase activity. These experiments involved growing adult primary fibroblast cells and HFF cells on microscope slide coverslips placed into each well of six well tissue culture plates. Each well containing a coverslip was seeded with 1.5×10⁵ cells and after one week of culture, the cells were treated with a range of proteins having peroxidase activity for up to 7 days. At the end of the experiment, the cell media was removed and the cells and cell-associated ECM fixed by the addition of 10% formalin. The coverslips were removed from the tissue culture plate and stained immunohistochemically for collagen I using the method outlined in Example 3 for the SMA immunohistochemistry with the variation that 5 μg/ml rabbit anti Collagen-I (Rocklands Immunochemicals) was used as the primary antibody.

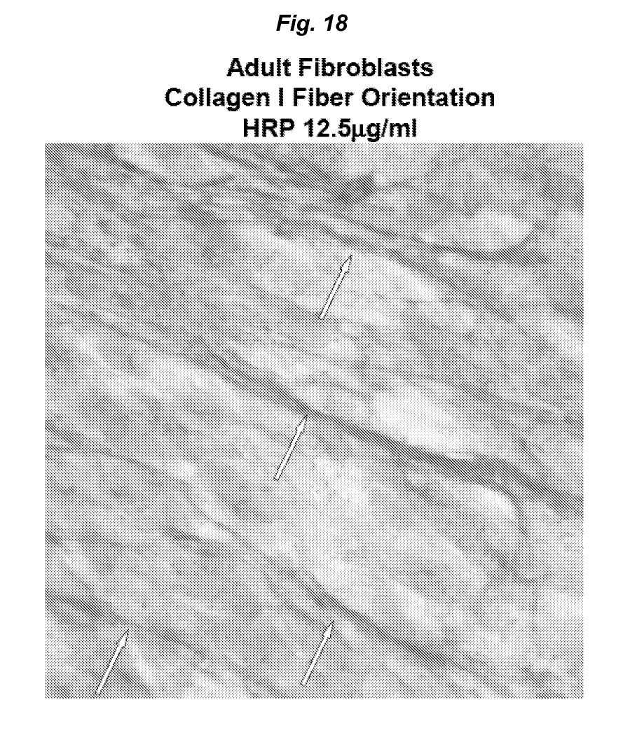

The stimulation of ECM production by human fibroblast cells with each protein having peroxidase activity resulted in the generation of strongly stained, parallel collagen fibers as highlighted by the arrows in FIG. 18 (HRP shown). Distinct from the other proteins having peroxidase activity, the staining pattern observed in cell monolayers treated with microperoxidase suggests collagen was not the major ECM protein deposited, although collagen fibers were evident. It will be appreciated by those skilled in the art that parallel, organised collagen fibers are indicative of well organised ECM showing the capacity of proteins having peroxidase activity to produce replacement tissue that is structurally and functionally consistent with normal tissue, rather than disorganised ECM which is often associated with, and used to define, scar tissue that has functional, structural and cosmetic deficits.

### Example 6

Proteins with Peroxidase Activity Elute from a Three-Dimensional Matrix and Stimulate ECM Production

Experiments were performed to determine if proteins with peroxidase activity absorbed into a 3-D matrix could be released or eluted from the 3-D matrix and stimulate fibroblast cells to produce ECM.

These experiments were performed by pre-incubating 1 cm×1 cm pieces of INTEGRA® with SBP (25 μg/ml) as described in Example 3 for either 30 minutes or 16 hours, control pieces were pre-incubated in DMEM only. After this time, the pieces of INTEGRA® were washed in fresh media to remove excess SBP solution, and placed into the wells of a 12-well culture plate pre-seeded with HFF cells (2×10⁵cells/well) that were grown to confluency over three days. The media from these wells was collected after 24 and 48 hours with peroxidase activity measured (to determine the rate of elution of the proteins with peroxidase activity from the Integra) and the collagen content measured by ELISA (to ensure the eluted proteins with peroxidase activity retained the capacity to stimulate collagen production by the HFF cells). The peroxidase activity was assessed using the Sigmafast™ O-phenylenediamine dihydrochloride (OPD) detection method as per the manufacturers instructions. Briefly, 5 μl of sample was combined with 100 μl of reagent and relative peroxidase activity determined after 5-10 minutes incubation by measuring absorbance at 450 nM using a Wallac Victor Multilabel HTS counter.

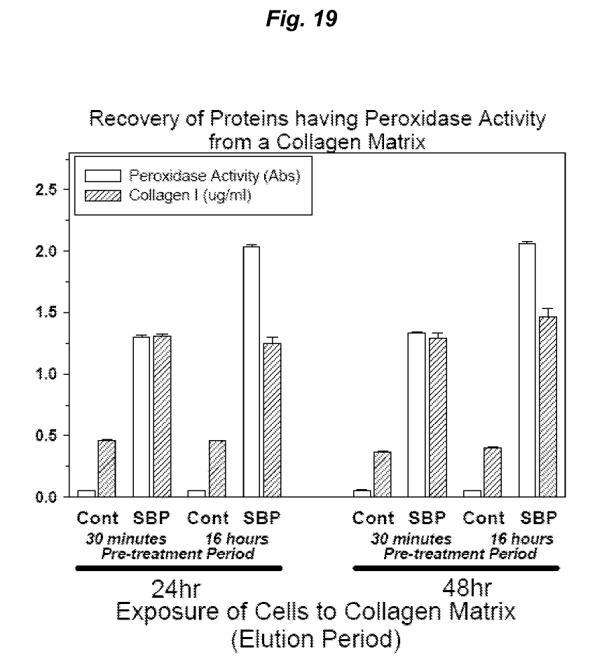

FIG. 19 surprisingly demonstrates that SBP pre-absorbed into the INTEGRA® matrix was eluted from the matrix into fresh medium bathing HFF cells. The level of peroxidase activity measured in the medium collected after 24 and 48 hours was identical indicating that the maximum amount of elution occurred within the first 24 hours. Even more surprisingly, the results shown in FIG. 19 demonstrate that the protein with peroxidase activity that diffused out of the INTEGRA® was able to stimulate ECM production (as measured by collagen I ELISA) by HFF cells exposed to the media containing the INTEGRA® pieces (control pieces of INTEGRA® not containing pre-absorbed SBP had no effect on basal collagen secretion). The amount of collagen I produced was similar after 24 and 48 hours, indicating that the amount of protein with peroxidase activity eluted from the INTEGRA® during the first 24 hours was sufficient to stimulate maximal ECM production. FIG. 19 also demonstrates that the level of peroxidase activity eluted from the INTEGRA® was approximately one third more in the media from INTEGRA® pieces pre-incubated for 16 hours compared to those pre-incubated for 30 minutes. This result indicates that more SBP was absorbed into the collagen matrix over 16 hours compared to 30 minutes. Despite this, sufficient SBP was obviously absorbed over 30 minutes and eluted to stimulate an amount of ECM production equivalent to that observed when SBP was absorbed over 16 hours.

### Example 7

HRP Conjugated Antibody Mediated Collagen I Production

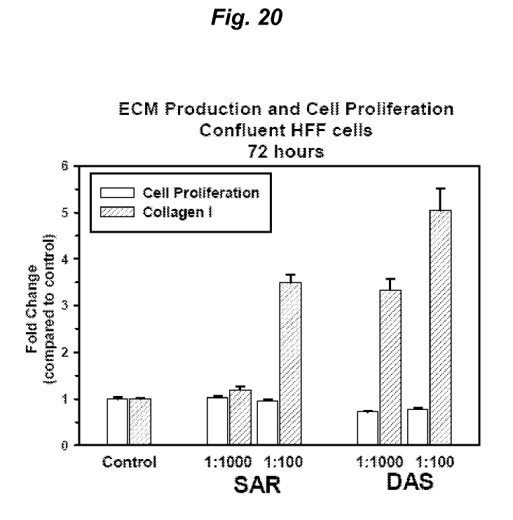

The methods of the experiment as outlined in Example 1 were repeated but with horseradish peroxidase protein (HRP) conjugated/coupled to a donkey anti-sheep/goat antibody (DAS) or a sheep anti-rabbit antibody (SAR) as the source of the protein with peroxidase activity. Triplicate wells of HFF cells were treated for six hours with a 1:100 or 1:1000 dilution of HRP coupled antibody and the concentration of soluble collagen I levels in the HFF conditioned media were measured and determined as described in Example 1. FIG. 20 shows that a 6 hour treatment with HRP conjugated antibody's stimulated a dose-dependent increase in collagen levels. The maximum increase stimulated by the 1:100 dilution of HRP coupled SAR or DAS was approximately 3.5 and 5-fold respectively over untreated, control cells and there was no concomitant increase in cell number, suggesting the HRP conjugated antibody's had a direct effect on collagen levels.

### Example 8

Proteins with Peroxidase Activity Induce a Fibrogenic Response in vivo

Experiments were performed to confirm proteins with peroxidase activity could induce a “fibrogenic response” when injected directly into the dermis. Such a response, characterised by an increase in fibroblast numbers and associated ECM deposition, is recognised as being a fundamental event necessary for wound healing. As such, an agent with the capacity to promote a fibrogenic response within the un-damaged dermis would also have the capacity to promote the regeneration and repair of dermal tissue and ultimately the healing of dermal wounds. In addition, an agent with the ability to promote a fibrogenic response within the un-damaged dermis would also have the capacity to stimulate generation of new dermal tissue and ultimately, promote the augmentation of dermal structures in un-damaged dermis.

Groups of up to 4-8 female Sprague-Dawley rats were lightly anaesthetized and the dorsum shaved. At different sites on their dorsum, each rat received a bolus, intradermal injection of 4 doses of a protein with peroxidase activity (LP, 5-125 n; HRP, 1.25-25 μg; SBP, 1.25-25 μg; or micro-peroxidase [MP], 5-125 μg), a vehicle control or TGFβ2 as a positive control. Arthromyces ramosus peroxidase (ARP) was also injected at one dose of 25 μg. Proteins with peroxidase activity were injected in a volume of 50 μl which was sufficient to create a small blister in the skin that resolved within a few minutes. Across the group of rats, each dose of proteins with peroxidase activity was injected into each of the different injection sites to control for any site-related, intra-rat variation. The rats were returned to their cage and left alone for 3 days after which they were killed by CO₂ asphyxiation and the skin at the injection sites collected for analysis. The skin at the injection sites was excised using a 6 mm biopsy punch with each piece bisected and fixed in 10% buffered formalin. Fixed tissue was processed for histological assessment by graded dehydration and mounting in paraffin blocks. Blocks were sectioned and sections stained with haematoxylin and eosin (H&E) and Masson's trichrome for qualitative analysis by an experienced pathologist. H&E stained sections were scored for the “fibroblast reaction” (1+-4+) to reflect the number of fibroblast cells identified at the injection site, with 4+ representing the greatest response. H&E and Massons trichrome stained sections were used to evaluate the amount of deposited, collagen-rich extracellular matrix (ECM) that was associated with the fibroblast reaction at each injection site. The collagen-rich ECM deposition was scored 1+-3+ with 3+ representing the greatest response. The scores were combined across the group of rats and statistical analysis performed using a Kruskal-Wallis one way analysis of variance on ranks (ANOVA) followed by post-hoc t-test comparing the response of the proteins with peroxidase activity to the control using Dunn's or Dunnett's method, p<0.05 was considered significant.

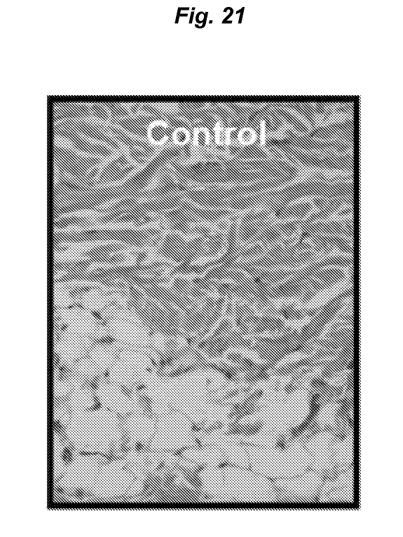

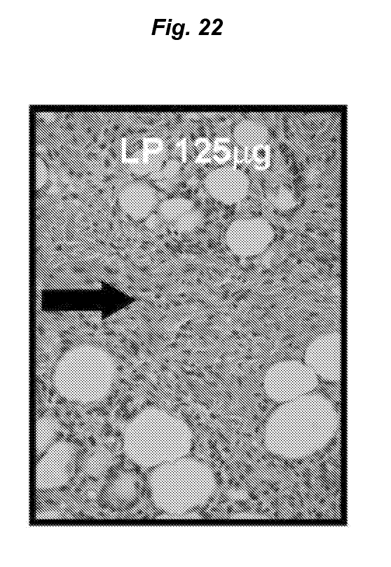

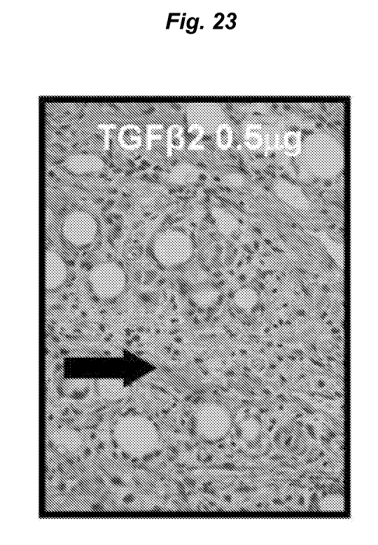

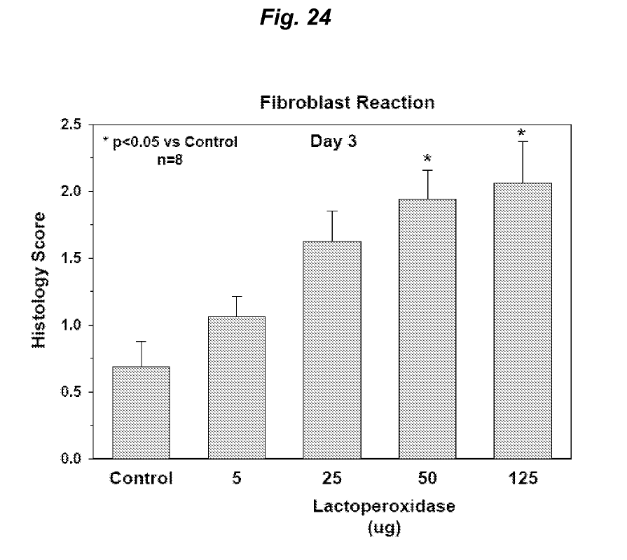

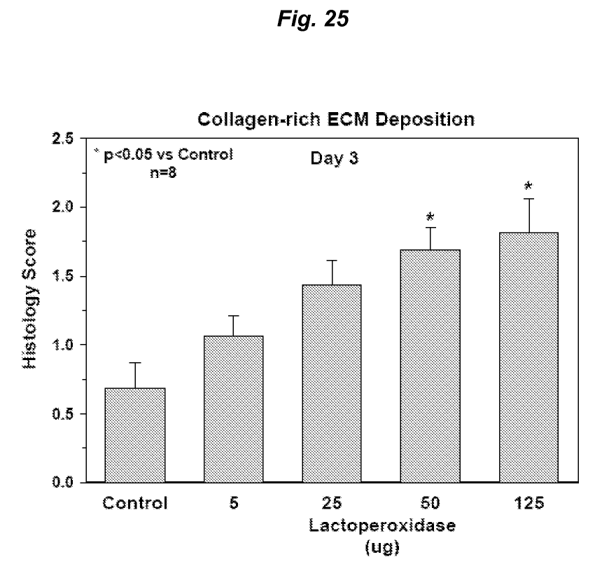

FIGS. 21 to 23 show representative images taken at x20 magnification of H&E stained sections from skin collected 3 days after injection with; 1, vehicle control (50 μg bovine serum albumin; BSA); 2, lactoperoxidase (LP 125 μg); 3, TGFβ32 (0.5 μg). The images show that there was no fibroblast reaction at the control-injected site (FIG. 21) which looked the same as adjacent, normal skin. Skin injected with LP (FIG. 22) or TGFβ3 (FIG. 23), showed a profound increase in the number of fibroblasts present at the injection site. The arrow shows an example of the fibroblast reaction in these sections, with the response generally noted to be localized to the dermis and panniculus adiposus (subcutaneous adipose tissue) and in some cases extend into the panniculus carnosus (subcutaneous muscle). The large, unstained areas are adipocytes. The collated scores from 8 animals (FIG. 24) show that LP induced a dose-dependent increase in the fibroblast reaction compared to the vehicle control with a statistically significant response observed when 50 μg and 125 μg of LP was injected (p<0.05). Similarly, a dose-dependent effect of LP on the deposition of collagen-rich ECM by the fibroblasts was observed (FIG. 25), with the injection of 50 μg and 125 μg LP resulting in a significant increase compared to the control (p<0.05). These results clearly show that LP stimulated mobilization of fibroblast cells to the site of injection and a fibroblast-associated increase in collagen-rich ECM production in vivo.

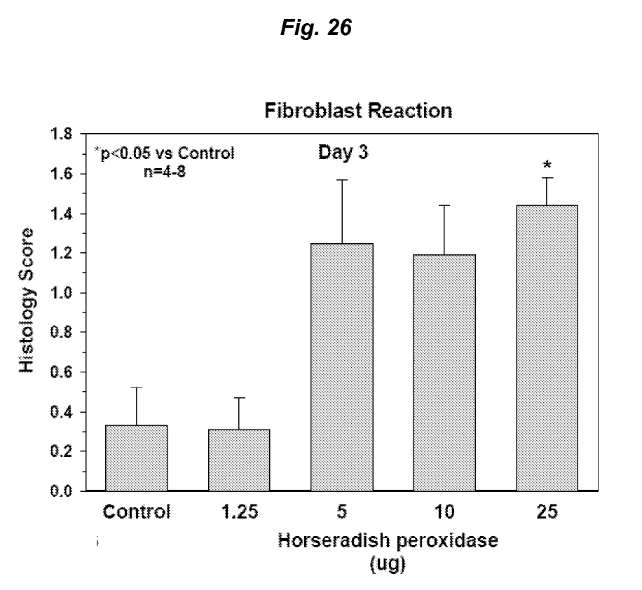

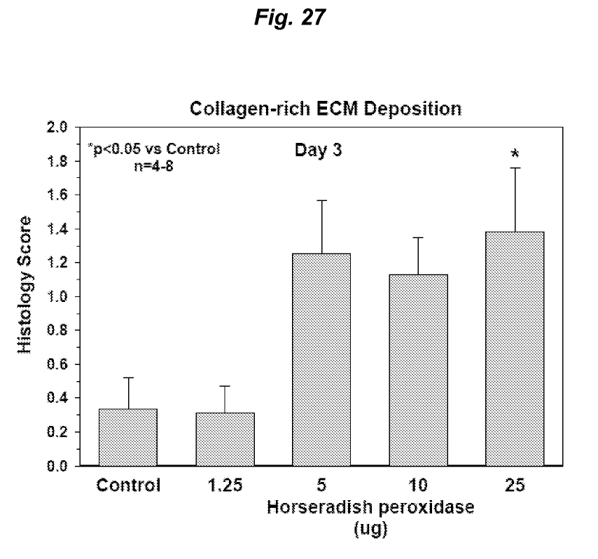

When horseradish peroxidase (HRP; 5-25 μg) was injected into the skin of rats, it was also found to have a fibroblastic effect after three days. The collated scores representing the fibroblast reaction and the deposition of collagen-rich ECM from 4-8 rats injected with HRP are shown in FIGS. 26 and 27 respectively. Compared to control, HRP markedly increased the tissue fibroblast content at the injection site and the deposition of collagen-rich ECM with a statistically significant response observed when 25 μg of HRP was injected (p<0.05). These results clearly show that HRP stimulated mobilization of fibroblast cells to the site of injection and a fibroblast-associated increase in collagen-rich ECM production in vivo.

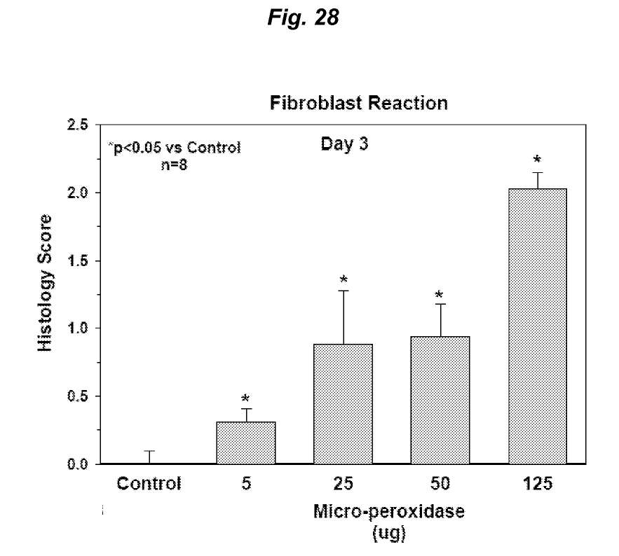

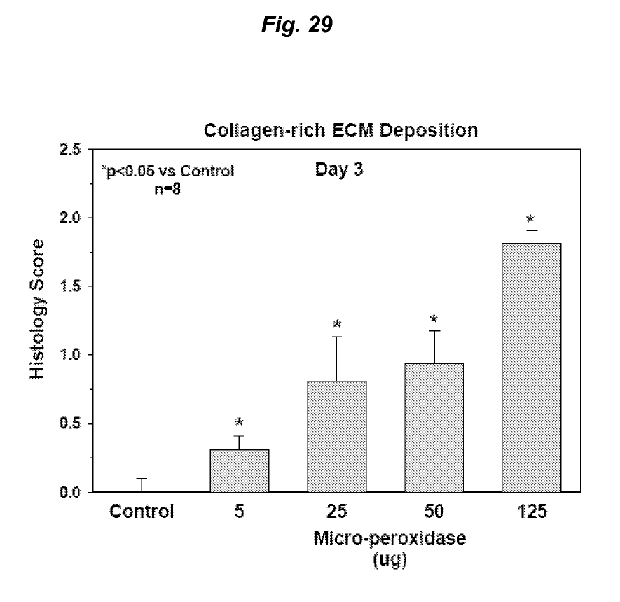

Microperoxidase (MP) injected into the skin of rats induced a fibroblastic response after 3 days at doses between 5 and 125 μg. FIG. 28 shows the collated scores representing the fibroblast reaction from 8 rats. At all doses used, MP significantly increased the tissue fibroblast content at the injection site compared to controls (p<0.05). FIG. 29 demonstrates that MP also stimulated the deposition of collagen-rich ECM in a significant, dose dependent manner (p<0.05). These results clearly show that MP stimulated mobilization of fibroblast cells to the site of injection and a fibroblast-associated increase in collagen-rich ECM production in vivo.

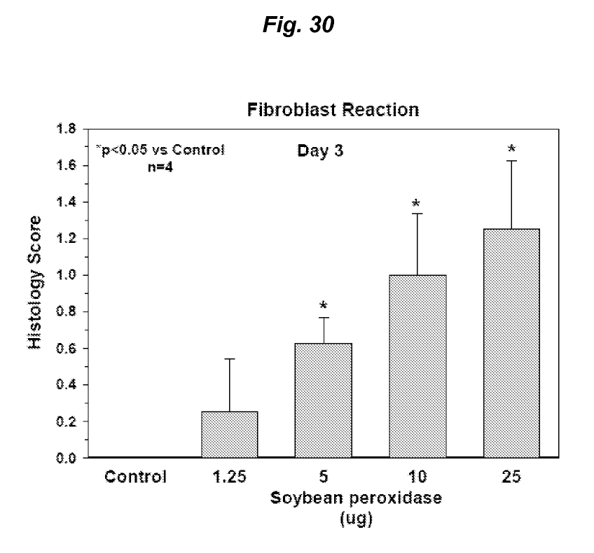

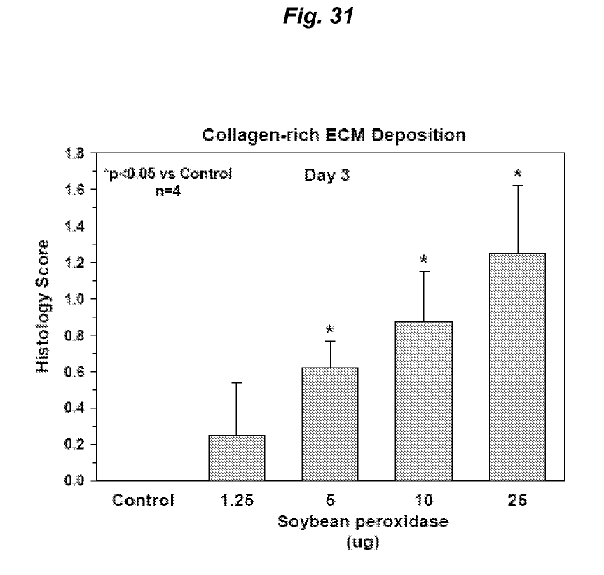

When soybean peroxidase (SBP; 1.25-25 μg) was injected into the skin of rats, it was also found to have a fibroblastic effect after three days. The collated scores representing the fibroblast reaction and the deposition of collagen-rich ECM from 4 rats injected with SBP are shown in FIGS. 30 and 31 respectively. Compared to control, SBP markedly increased the tissue fibroblast content at the injection site and the deposition of collagen-rich ECM with a statistically significant response observed when 5-25 μg of SBP was injected (p<0.05). ARP at the representative dose tested (25 μg) was found to have an at least equivalent effect as the other proteins with peroxidases activity on both the fibroblast reaction and the deposition of collagen rich ECM, with a histology score of two recorded for both responses after 72 hours. These results clearly show that SBP and ARP stimulated mobilization of fibroblast cells to the site of injection and a fibroblast-associated increase in collagen-rich ECM production in vivo.

### Example 9

Proteins with Peroxidase Activity Promote Cellular Integration and ECM Production in vivo when Combined with a Substrate

The ability of agents with peroxidase activity to stimulate the production of endogenous ECM within, or about, a substrate when implanted in vivo was assessed. Substrates used for these experiments were two hyaluronic acid based dermal filler substances; Hylaform® Plus containing 5.5 mg/ml hylan B gel and Restylane® containing 20 mg/ml stabilised hyaluronic acid, and Sculptra® containing poly-L-lactic acid. The two pre-prepared “ready to use” hyaluronic acid filler substances were diluted 1:2 with 1 mg/ml HRP to give a final concentration of 500 μg/ml HRP whilst Sculptra powder was dissolved in a lmg/ml LP solution to final concentrations of 25 and 50 mg/ml poly-L-lactic acid. As described in example 8, female Sprague-Dawley rats were lightly anaesthetized and a volume of approximately 50 μl of the filler substance+protein with peroxidase activity or filler substance alone were injected intradermally into different sites on their dorsum. The rats were returned to their cage and left alone for 7 days or 1 month after which they were killed by CO₂ asphyxiation and the skin at the injection sites collected for histological assessment as described in example 8, or immunohistochemical staining for collagen I using the method outlined previously for the SMA immunohistochemistry (in Example 3) with the variation that 5 μg/ml rabbit anti Collagen-I (Rocklands Immunochemicals) was used as the primary antibody.

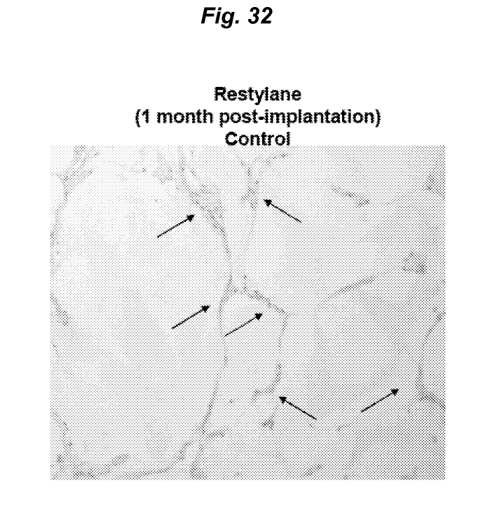

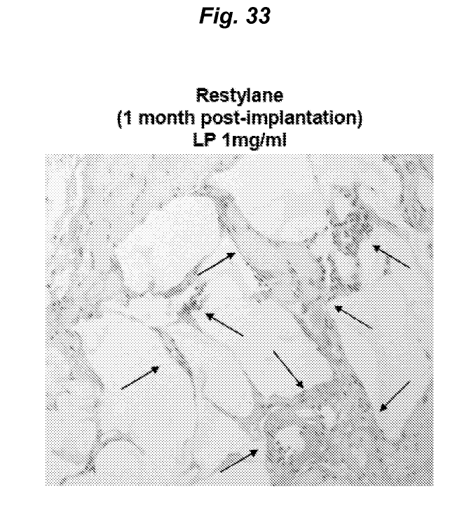

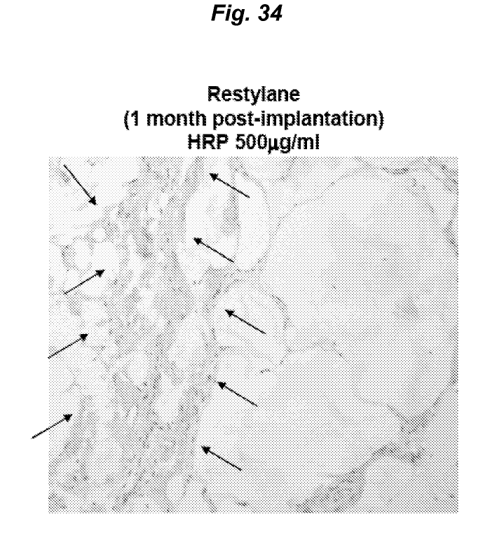

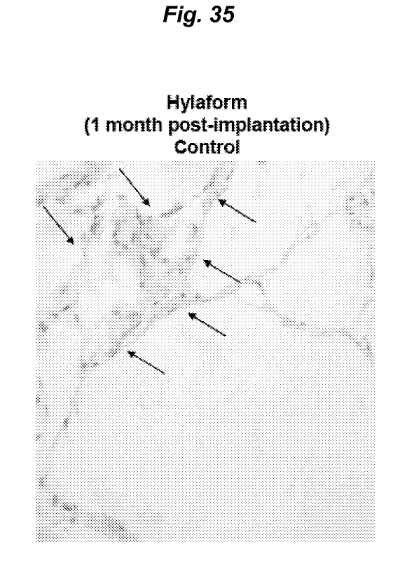

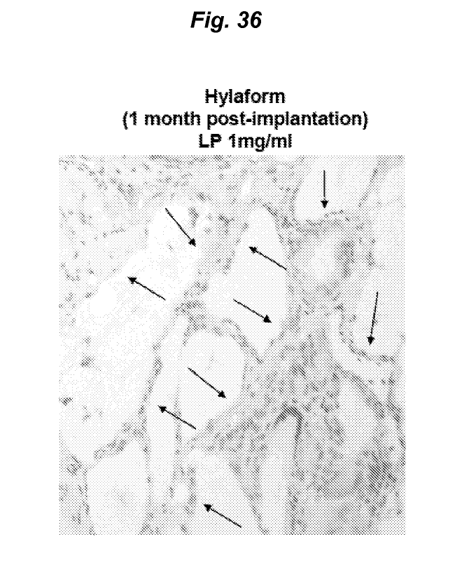

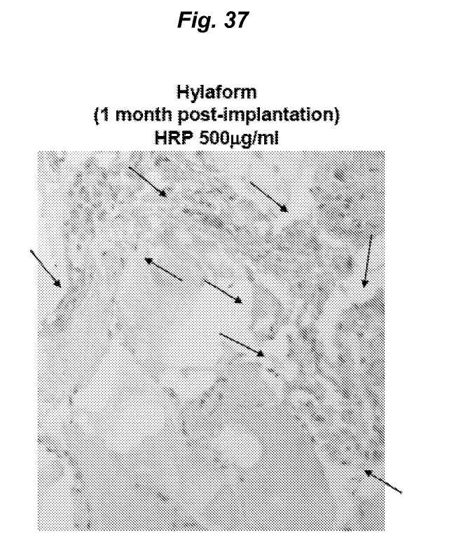

FIG. 32 shows the degree of cellular integration and ECM production that can be expected one month after Restylane (20 mg/ml stabilised hyaluronic acid) alone is implanted within the dermis. The arrows show the limited areas of cell-associated ECM deposited around lakes of the hyaluronic acid gel. In contrast, FIG. 33 shows the surprising increase in cellular infiltration and integration and cell-associated ECM production stimulated when LP was added to the hyaluronic acid gel prior to injecting it into the dermis. Increased cellular integration and ECM production was also observed when HRP was added to the Restylane prior to implantation (FIG. 34). Similar results were observed when Hylaform (5.5 mg/ml Hylan B) was used as the primary filler substance. FIG. 35 shows the degree of cellular integration and ECM production that can be expected one month after Hylaform alone is implanted within the dermis. The arrows show the limited areas of cell-associated ECM deposited around lakes of the Hylaform gel. In contrast, FIG. 36 shows the surprising increase in cellular infiltration and integration and cell-associated ECM production stimulated when LP was added to the Hylaform gel prior to injecting it into the dermis. Increased cellular integration and ECM production was also observed when HRP was added to the Hylaform gel prior to implantation (FIG. 37).

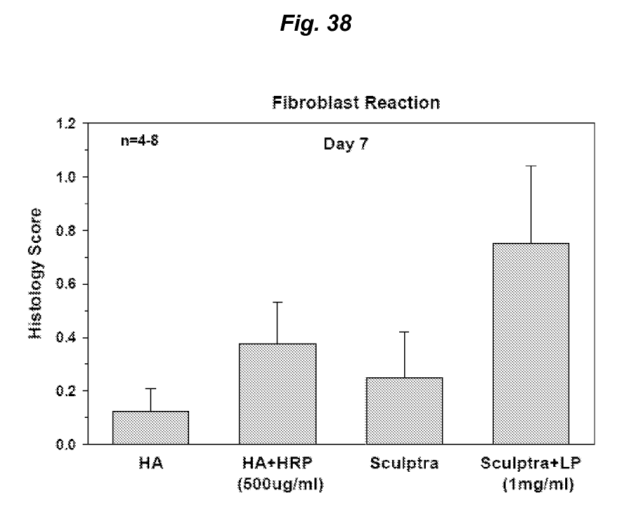

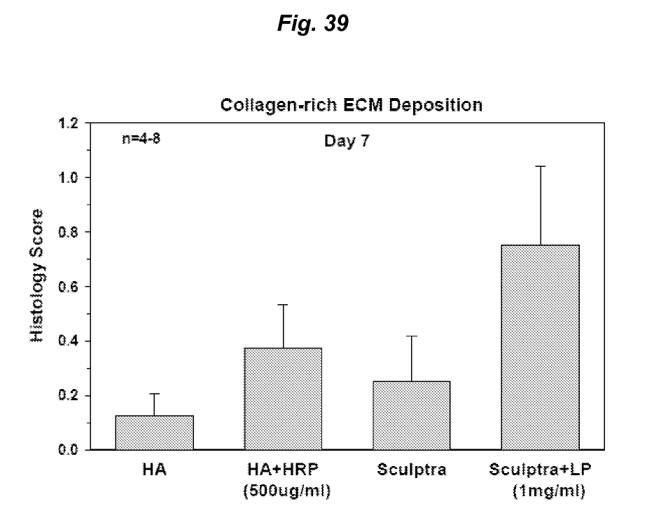

FIGS. 38 and 39 show the fibroblast reaction and ECM deposition observed 7 days after the implantation of either the hyaluronic acid or the poly-L-lactic acid filler substances+/−proteins with peroxidase activity. The combined data from sites injected with both forms of hyaluronic acid (Restylane and Hylaform) is shown. FIG. 38 demonstrates combining proteins with peroxidase acitivity with the filler substances increased the fibroblastic response within or about the implanted material. FIG. 39 shows the addition of a protein with peroxidase activity to the two different types of filler substances also increased the deposition of collagen-rich ECM within or about the implanted material after 7 days

These results confirm proteins with peroxidase activity can promote the generation of new fibroblast-derived tissue in vivo when provided within a substrate acting as a dermal filler substance. In addition, these results confirm proteins with peroxidase activity can promote the generation of new fibroblast-derived tissue in vivo when provided within a substrate or three-dimensional matrix capable of acting as a tissue regeneration template.

### Example 10

Proteins with Peroxidase Activity Promote a Fibrogenic Response in Human Skin

To confirm proteins with peroxidise activity can stimulate ECM production in human skin, studies were performed on ex vivo skin collected from tissue reduction surgery. The studies were undertaken using a modification of the OECD Test Guideline 428 (2004) Skin Absorption: in vitro method. Freshly excised human skin was collected in a

Hanks balanced salt solution (HBSS) and after being thoroughly washed in sterile, colour free DMEM/F12 media (Dulbecco's Modified Eagle Medium/Hams F12, Invitrogen) to remove surface debris, the subcutaneous fat layer was removed. Circles of full-thickness skin 15 mm in diameter were cut and placed epidermis side up on vertical glass diffusion cells of a Franz cell diffusion system (Crown Glass Co, NJ, USA). The skin was secured between two clamped ground glass joints and allowed to equilibrate and hydrate for approximately 30 minutes with the receptor chambers of the diffusion cells containing colour-free DMEM/F12+1% (v/v) of an antibiotic mix (200 mM L-glutamine, 10,000 units penicillin, 10 mg streptomycin per ml; Sigma, USA). The temperature of the jacketed diffusion cells was maintained at 34° C.±1° C. and the contents were constantly stirred using a mini magnetic stirrer. Proteins with peroxidase activity or a control were injected intradermally into the skin discs in a volume of 100 μl using a 27 gauge needle. After 72 hours the skin discs were removed and bisected with one half fixed in 4% buffered formalin for histological assessment and the other half snap frozen in liquid nitrogen for immunohistochemical detection of collagen I and III protein within the dermis. Frozen skin pieces were processed into cryoblocks, cut onto slides and fixed in acetone/methanol. All slides were blocked with 2% (w/v) bovine serum albumin (BSA) in HBSS and then incubated with either anti-human collagen type I or III primary antibodies (Chemical Credential, ICN or DakoCytomation respectively). The secondary antibody was biotinylated Multi-Link (DakoCytomation) with detection by streptavidin-biotinylated alkaline phosphatase (DakoCytomation). Visualisation was achieved by incubating each section with Naphthol AS/MX phosphate/fast red substrate containing 5 mM levamisole (Sigma). After washing, slides were counter stained with Mayer's Haematoxylin and assessed for collagen expression.

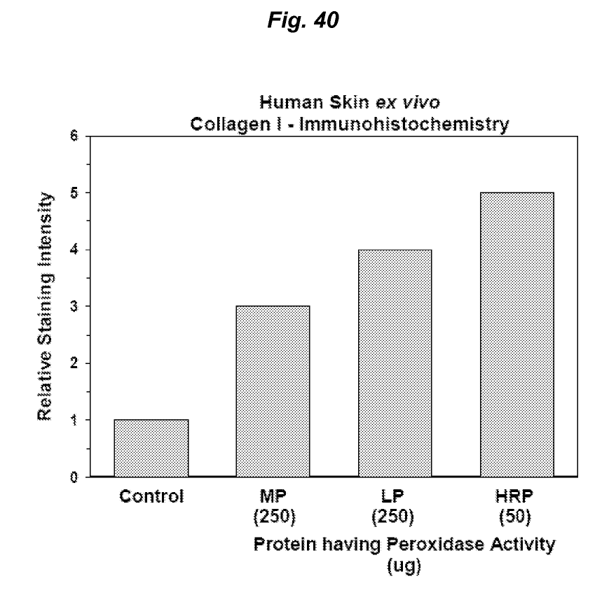

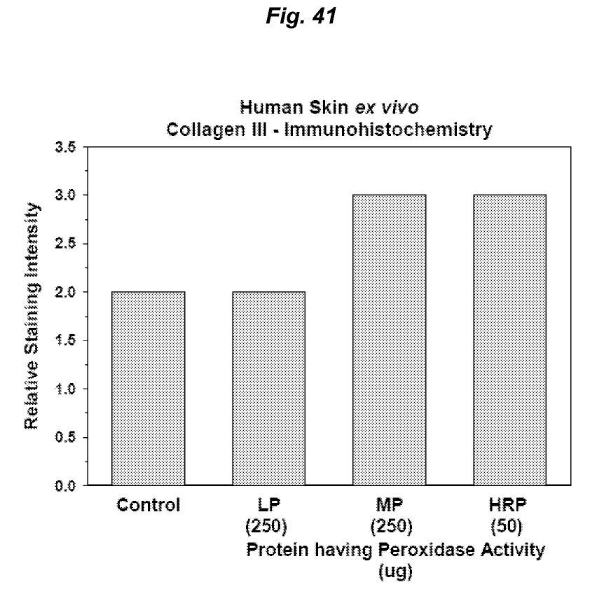

Sections stained for collagen I and collagen III were assessed by an independent, blinded observer who rated the sections based on their relative levels of immunoreactivity as +1 (weak staining) to +5 (strong staining) Immunohistochemical staining for collagen I showed MP, LP and HRP increased staining intensity for collagen I (FIG. 40) which was noted to be predominantly localised in the mid to upper papillary dermis and the upper reticular dermis. The results in FIG. 41 also show that whereas LP injected into the dermis did not appear to affect collagen III immuno-reactivity, both MP and HRP increased collagen III staining

### Example 11

Proteins with Peroxidase Activity Protect HFF Cells from the Inhibitory Effects of corticosteroids on Collagen I Production

Corticosteroids such as dexamethasone, prednisolone, betamethasone, and hydrocortisone are well known to suppress collagen synthesis by fibroblasts and other collagen producing cells by inhibiting TGF-beta-mediated collagen induction. When corticosteroids are used medically as anti-inflammatory or immunosuppressive agents at high doses or over an extended period of time, a number of adverse side effects can arise such as dermal atrophy and osteoporosis (brittle bones). Moreover, medical conditions such as Cushing's syndrome are characterized in part by detrimental skeletal and connective tissue symptoms due to increased circulating levels of endogenous steroid hormones like cortisone. Experiments were performed to determine whether proteins with peroxidase activity have the ability to protect fibroblast cells from the inhibitory effect of dexamethasone on collagen synthesis and thus provide a way of overcoming the detrimental effects on skin and other ECM rich tissues of corticosteroid agents.

In these experiments cells were pre-treated with dexamethasone (20 μM) during the O/N serum starvation period with dexamethasone also applied to the cells during the 6 hour treatment period and the ensuing 72 hr culture period. Controls received no dexamethasone.

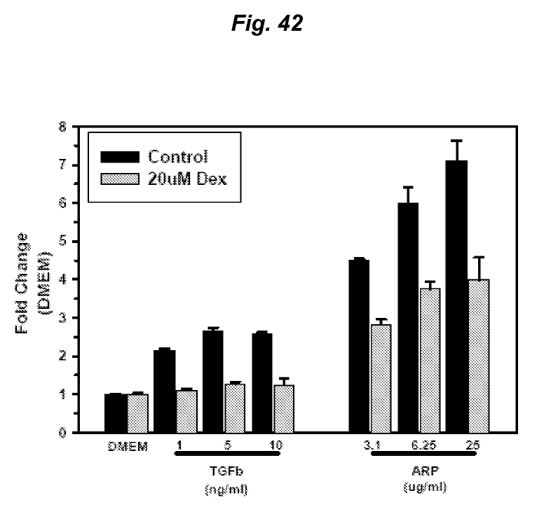

FIG. 42 shows the control response of HFF cells to the fibrogenic factor transforming growth factor β2 (TGFβ2). TGFβ2 at doses ranging from 1-10 ng/ml induced a maximum 2-2.5-fold increase in collagen I levels, a result consistent with a range of studies demonstrating the effect of TGF-beta on collagen production (reviewed by Grande, P.S.E.B.M 1997, 214, 27-40). FIG. 42 also shows the comparatively greater response obtained when cells were exposed to ARP (as a representative protein with peroxidase activity) which stimulated a 4.5-7 fold increase in collagen I levels when used at doses ranging from 3.1-25 μg/ml.

FIG. 42 also shows that dexamethasone totally inhibited collagen induction by cells treated for 6 hours with TGFβ2. Although dexamethasone also inhibited collagen stimulated by ARP by approximately 30%, significant levels of collagen accumulation were still evident in cells treated with both dexamethasone and ARP. These results demonstrate that proteins with peroxidase activity are able to spare fibroblasts from the collagen inhibitory activity of steroid hormones like dexamethasone. Furthermore, the methods and compositions of the invention, comprising a protein with peroxidase activity can be of utility in subjects with Cushing's syndrome or as a co-therapy with steroid treatment in order to at least partially reduce steroid mediated ECM/collagen loss. Alternatively the methods and compositions of the present invention may be used in the recovery from steroid-induced ECM damage.

### Example 12

Tissue Regeneration Templates in vivo

The examples demonstrate that proteins with peroxidase activity stimulate fibroblast cells to populate the interstices of a porous collagen-based three dimensional scaffold much quicker and more effectively than untreated cells when seeded thereon in vitro. The examples also demonstrate that including agents with peroxidase activity within a three-dimensional ECM-derived scaffold transplanted in vivo, promotes the migration of fibroblast cells into the structure of the three-dimensional scaffold and the production of ECM by the fibroblast cells.

To demonstrate that proteins with peroxidase activity can greatly enhance the function of tissue regeneration templates and devices that are used clinically to replace lost tissue and to aid healing and repair, the following studies would be performed. Examples of suitable tissue regeneration templates and devices that can be used in conjunction with proteins with peroxidase activity include:
Collagen matrix wound dressings or wound dressings composed of other ECM proteins
Synthetic polymer scaffolds
Temporary skin replacements such as Biobrane®/Biobrane-L®
Acellular dermal regeneration templates (including allografts and xenografts) such as INTEGRA® Dermal Regeneration Template (DRT) and INTEGRA™ Bilayer Matrix Wound Dressing (BMWD), AlloDerm®, GRAFTJACKET®, GammaGraft®, Oasis®, EZ Derm™
Allogeneic Dermal Substitutes such as Dermagraft® and Transcyte
Allogeneic Skin Substitutes such as Apligraf® and Orcel™
Split-thickness autografts

Proteins with peroxidase activity will be incorporated into the scaffold or framework prepared as a tissue regeneration template, either during the manufacture of the template or at the time of application of the template to the wound. For example, the tissue regeneration template (or autograft) will be hydrated in a solution containing a protein with peroxidase activity (at a range of concentrations from 1 μg/ml-1 mg/ml) for a range times from one minute to sixteen hours prior to the application of the template to the wound. In addition, some wounds will also be irrigated with a solution containing a protein with peroxidase activity prior to the application of the template and/or at regular intervals post-application of the template such as at dressing changes.

Wounds will be partial or full-thickness dermal wounds caused by the excision of the dermal and epidermal layers using a scalpel or dermatome for example, or by application of a heat source resulting in a burn. Where burn wounds are created, the area of dead and damaged skin is surgically excised or “debrided” following standard clinical practice. Wounds will be created on the dorsal aspect of an animal, such as a human, pig, sheep, rabbit, guinea pig, rat or mouse after the skin has been adequately prepared ie hair removed by shaving and skin washed with a suitable antiseptic agent such as povidone-iodine and 70% isopropanol. The tissue regeneration template of choice (+/− protein with peroxidase activity) will be applied to the wound and where necessary, fixed to the surrounding undamaged skin by sutures or staples following standard clinical practice. In a variation of the standard experiment, the selected tissue regeneration template (or autograft) will be meshed to increase the surface area covered by the graft following standard clinical practice. At least paired wounds will be created on each animal such that one wound acts as a control wound and is treated with the selected tissue regeneration template without the addition of proteins with peroxidase activity. Each wound will then covered by a secondary dressing as recommended by the manufacturers instructions for the template (ie absorbent dressing), with the dressings changed at regular intervals following standard clinical practice and animals treated with prophylactic antibiotics as necessary.

Once a viable neodermis has formed with an adequate level of vascularisation within or about the tissue regeneration template, a subset of wounds will be treated with cultured epithelial autografts (CEA) or split-thickness autografts following standard clinical practice, with another subset of wounds allowed to proceed to wound closure without additional intervention. The CEA's will be applied as cell sheets generated by in vitro cell culture or as a cell suspension that will be sprayed onto the neodermis of the tissue regeneration template. CEA's will be prepared by removing a split-thickness skin sample from the animal and removing the epidermis from the dermis by enzymatic or mechanical means. The epidermal cells are then dispersed and propagated in cell culture to expand the population and provide sufficient cells to apply to the wound. Preparations of CEA's are well known in the art, and the skilled practitioner will know of many variations, with all such variations that can be used to seed a wound treated with a dermal regeneration template within the scope of this invention. It will also be recognise by those skilled in the art that wounds treated with different tissue regeneration templates require different preparation prior to the application of CEA's or split-thickness autografts. Wounds treated with a product like INTEGRA® will have the temporary silicone layer or “artificial” epidermis removed whilst wounds treated with products derived from cadaveric skin (ie Graftjacket and Alloderm) will have the epidermal layer of the product removed by a dermatome. Following application of the CEA or split-thickness autograft, the wounds will be dressed with a suitable dressing such as paraffin-coated gauze, petroleum impregnated gauze or an occlusive dressing such as Opsite and protected with additional gauze and appropriate bandaging.

Following placement of a tissue regeneration template onto a wound, the wound will be examined regularly (ie every 1-2 days) to qualitatively determine the degree of engraftment (ie adherence of the template to the wound bed) and time taken for a viable neodermis to form within or about the template. Biopsies will also be collected regularly from within the grafted template to determine the fibroblastic or “tissue integration” response evident within the template (ie degree of cellular infiltration and ECM production) using histological and immunohistological methods described in examples 5 and 7. The qualitative assessments will be extended as healing progresses and some wounds are grafted with CEA's or split-thickness autografts to include endpoints such as sub-graft exuadate formation, coloration, keratinisation, percent of wound covered, erythema, pigmentation, epidermal blistering, external surface quality, skin suppleness and raised scar.

It is expected these studies will show that the addition of a protein with peroxidase activity to the tissue regeneration template will reduce the time required for the template to become integrated with the wound bed and also improve the overall degree and quality of the engraftment or attachment. In addition, it is expected that the presence of a protein with peroxidase activity will accelerate the infiltration of the template by fibroblast cells and the subsequent deposition of ECM within the interstices of the template and thus promote the vascularisation of the template so that it can support the attachment of CEA's or split-thickness autografts much sooner than templates applied without the addition of proteins with peroxidase activity. It also expected the proteins with peroxidase activity will enhance the graft take of the CEA's and split-thickness autografts due to the improved quality of the neodermis promoting the formation of a viable basement membrane between the neodermis and neoepidermis. In addition, it also expected the tissue regeneration templates treated with proteins with peroxidase activity to promote the reepithelialisation of the wound and therefore wound closure or healing, even without the application of CEA's or split-thickness autografts. As such, these outcomes will provide clinical benefits in reducing the time required for the wounds to heal and thus reduce patient mortality and morbidity and reduced health care costs. In addition, replacement of connective tissue in excised, full-thickness dermal wounds, such as that stimulated by proteins with peroxidase activity, is well recognised in the art as a fundamental requirement for restoration of skin function and cosmesis. Thus the addition of proteins with peroxidase activity to tissue regeneration scaffolds used in vivo will improve the surface texture and suppleness of the skin, reduce the occurrence of epidermal ulceration and maceration and will greatly improve the mechanical robustness of the regenerated tissue.

### Example 13

Generation of Three-Dimensional Tissue Substitutes in vitro for Use in vivo

The examples demonstrate that agents with peroxidase activity stimulate fibroblast cells to populate the interstices of a porous collagen-based three dimensional scaffold much quicker and more effectively than untreated cells when seeded thereon in vitro. The invention can therefore be used to accelerate the generation of dermal replacements and skin equivalents in vitro, for subsequent application to a wound on an animal in need thereof. To ensure a dermal replacement or skin equivalent generated in vitro using proteins with peroxidase activity can function effectively as a tissue replacement device in vivo, the following studies would be performed.

INTEGRA® Dermal Regeneration Template (DRT) or another suitable substrate will be cellularised with human foreskin fibroblast (HFF) cells following the methods outlined in example 5 but at a much larger scale. In these studies sheets of INTEGRA® from 2 cm×2 cm to 10 cm×10 cm sizes will be inoculated with HFF cells at a seeding density of approximately 5×10⁵ cells/cm². After culturing for between 3 days and 2 weeks in media with or without the addition of a protein with peroxidase activity, the cellularised INTEGRA® will be harvested for application to a wound.

Wounds will be partial or full-thickness dermal wounds caused by the excision of the dermal and epidermal layers using a scalpel or dermatome for example, or by application of a heat source resulting in a burn. Where burn wounds are created, the area of dead and damaged skin will be surgically excised or “debrided” following standard clinical practice. Wounds will be created on the dorsal aspect of an animal, such as a human, pig, sheep, rabbit, guinea pig, rat or mouse after the skin has been adequately prepared ie hair removed by shaving and skin washed with a suitable antiseptic agent such as povidone-iodine and 70% isopropanol.

The cellularised INTEGRA® (+/− protein with peroxidase activity added at a range of concentrations from 1 μg-1 mg/ml) is applied to the wound and where necessary, fixed to the surrounding undamaged skin by sutures or staples following standard clinical practice. In a variation of the standard experiment, the INTEGRA® will be meshed to increase the surface area covered by the graft following standard clinical practice. At least paired wounds will be created on each animal such that one wound acts as a control wound and is treated with cellularised INTEGRA® cultured without the addition of proteins with peroxidase activity. Each wound will then be covered by a secondary dressing as recommended by the manufacturers instructions for INTEGRA® (ie absorbent dressing), with the dressings changed at regular intervals following standard clinical practice and animals treated with prophylactic antibiotics as necessary.

Following placement of the cellularised INTEGRA® (graft) onto a wound, the wound will be examined regularly (ie every 1-2 days) to qualitatively determine the degree of engraftment (ie adherence of the INTEGRA® to the wound bed) and the time taken for the graft to become vascularised. Biopsies will also be collected regularly from within the graft to determine the fibroblastic or “tissue integration” response evident within the template (ie degree of cellular infiltration and ECM production) and the degree of vascularisation and epithelialisation using histological and immunohistological methods described in examples 5 and 7. The wounds will be allowed to proceed to complete wound closure or healing, which will entail removal of the artificial epidermis (silicone layer) of the INTEGRA® at an appropriate time. The qualitative assessments will be extended as healing progresses to include endpoints such as sub-graft exuadate formation, coloration, keratinisation, percent of wound covered, erythema, pigmentation, epidermal blistering, external surface quality, skin suppleness and raised scar.

In a variation of the aforementioned experiment, once an adequate level of vascularisation has been achieved within the grafted, cellularised INTEGRA, a subset of wounds will be treated with cultured epithelial autografts (CEA) or split-thickness autografts following standard clinical practice. The CEA's will be applied as cell sheets generated by in vitro cell culture or as a cell suspension that will be sprayed onto the neodermis of the tissue regeneration template. CEA's will be prepared by removing a split-thickness skin sample from the treated animal and removing the epidermis from the dermis by enzymatic or mechanical means. The epidermal cells are then dispersed and propagated in cell culture to expand the population and provide sufficient cells to apply to the wound. Preparations of CEA's are well known in the art, and the skilled practitioner will know of many variations, with all such variations that can be used to seed a wound treated with a dermal regeneration template within the scope of this invention. Following application of the CEA or split-thickness autograft, the wounds will be dressed with a suitable dressing such as paraffin-coated gauze, petroleum impregnated gauze or an occlusive dressing such as Opsite and protected with additional gauze and appropriate bandaging. Both histological and qualitative assessments will be performed to determine the improvement in healing outcomes associated with preparing the cellularised INTEGRA® in the presence of proteins with peroxidase acitivity.

It is expected that these studies will show that a cellularised dermal replacement cultured in vitro in the presence of proteins with peroxidase activity will provide superior wound healing outcomes than a cellularised dermal replacement cultured for the same period of time but without the addition of proteins with peroxidase activity. The generation of the cellularised dermal replacement in the presence of a protein with peroxidase activity will reduce the time required for the template to become integrated with the wound bed and also improve the overall degree and quality of the engraftment or attachment. In addition, it is expected the superior population of the INTEGRA® template with fibroblast cells and subsequent deposition of ECM within the interstices of the template stimulated by the proteins with peroxidase activity in vitro, will accelerate the infiltration of the template by vascular cells in vivo and thus promote the epithelialisation of the dermal replacement so complete wound closure or healing can be achieved much sooner than dermal replacements generated without the addition of proteins with peroxidase activity. It is also expected the dermal replacement generated in the presence of proteins with peroxidase activity will more readily support the attachment of CEA's or split-thickness autografts and improve the graft take of the CEA's and split-thickness autografts due to the superior quality of the dermis promoting the formation of a viable basement membrane between the dermis and neoepidermis. As such, these outcomes will confirm using the method of the invention to optimise the generation of a dermal replacement or skin equivalent device in vitro will provide a product with clinical benefits such as reducing the time required for the wounds to heal with associated reductions in patient mortality, morbidity and health care costs.

### Example 14

Methodologies Used to Study the Ability of Proteins with Peroxidase Activity to Prevent or Ameliorate the Loss of ECM Induced by Steroid Therapy

The invention can be used to prevent or ameliorate the loss of ECM levels in skin and other organs resulting from the inhibition of TGF-beta -mediated ECM production (including collagen I) by topically or systemically administered corticosteroids, generally for the treatment of inflammatory conditions. Typically, the long term administration of high dose corticosteroid either topically or systemically leads to skin atrophy and impaired wound healing with this adverse side effect ultimately reducing the effectiveness of these treatments. The person skilled in the art is readily able to investigate the claimed invention to prevent or ameliorate corticosteroid-induced loss of ECM by way of the following examples.

(i) Topical Dermal Administration of Corticosteroids

To test the ability of proteins with peroxidase activity (HRP, SBP, MP and ARP etc) to prevent or ameliorate the loss of ECM in the skin caused by the topical administration of corticosteroids, the following experiment is performed. Anaesthetized adult rats are shaved to remove the hair and enable the skin to be freely accessed. Six 1 cm diameter circles are marked on the dorsum of each rat and effective doses of corticosteroid containing preparations (creams or ointments such as Elocon; mometasone furoate 1 mg/g, Antroquoril; betamethasone valerate equivalent to betamethasone 200 μg/g, Sigmacort; hydrocortisone acetate 10 mg/g) applied 1-2 times daily at the rate of approximately 0.01-0.1 g/cm² for two to four weeks. Effective doses of proteins with peroxidase activity (HRP, SBP, MP and ARP etc) ranging from 0.01-100 mg/g incorporated into a relevant carrier together with carrier only controls are also applied directly to the same skin treated with corticosteroid preparations at the rate of approximately 0.01-0.1 g/cm². The application of formulations containing proteins with peroxidase activity can occur prior to the application of corticosteroid preparations (eg 1-2 hours), at the same time or sometime thereafter (eg 1-2 hours). The animals are returned to their cage and housed under normal day/light conditions (12 hr/12 hr) with standard chow and water available ad libitum. At the end of the experiment, the animals are killed and the treated area of dermis excised from the surrounding skin. This skin sample is placed into 4 or 10% phosphate buffered formalin for fixation, mounted in paraffin and sectioned onto microscope slides at thicknesses up to 20 microns for histological staining and assessment. Histological examination would be performed. Additional replicate experiments are performed to collect tissue to extract for quantification of collagen I levels and for immunohistochemical identification of ECM proteins being produced in vivo.

(ii) Systemic Administration of Corticosteroids

To test the ability of proteins with peroxidase activity (HRP, SBP, MP and ARP etc) to prevent or ameliorate the loss of ECM in the skin caused by the systemic administration of corticosteroids, proteins with peroxidase activity are applied topically to the skin. In this experiment, anaesthetized adult rats are shaved to remove the hair and enable the skin to be freely accessed. The animals are injected with a long acting corticosteroid preparation (for example DepoMedrol; methylprednisolone) at a dose range between 10 and 30 mg/kg. Effective doses of proteins with peroxidase activity (HRP, SBP, MP and ARP etc) ranging from 0.01-100 mg/g incorporated into a relevant carrier, together with carrier only controls, are applied directly to the dorsal skin at the rate of approximately 0.01-0.1 g/cm². The application of formulations containing proteins with peroxidase activity will be performed 1-2 times a day and can occur prior to the injection of the corticosteroid preparation (eg starting 1-7 days before), from the time of the injection or sometime thereafter (eg starting 1-3 days after the injection). The animals are returned to their cage and housed under normal day/light conditions (12 hr/12 hr) with standard chow and water available ad libitum. The topical application of proteins with peroxidase activity will be performed for 3, 5, 7, 10 and 14 days after the injection of the corticosteroid preparation. The animals will then be killed and the treated area of dermis excised from the surrounding skin. This skin sample is placed into 4 or 10% phosphate buffered formalin for fixation, mounted in paraffin and sectioned onto microscope slides at thicknesses up to 20 microns for histological staining and assessment.

Histological examination is performed as usual. Additional replicate experiments will be performed to collect tissue to extract for quantification of collagen I levels and for immunohistochemical identification of ECM proteins being produced in vivo. The quantification of tissue levels of collagen I is performed using the ELISA method described herein with modifications to suit the extraction of collagen from whole skin as described by Robins et al (2003) J Invest Dermatol 121: 267-272.

It is expected that proteins with peroxidase activity stimulate the local production of ECM (including collagen I) at and around the application site despite the administration of corticosteroids either topically or systemically. Increasing the local tissue content of

ECM in such a way demonstrates the proteins with peroxidase activity provide the means to prevent, ameliorate or treat corticosteroid-induced skin atrophy or impaired wound healing resulting from the generalized inhibition by corticosteroids of TGF-beta mediated ECM production.

### Example 15

Compositions Useful in the Treatment of Skin Conditions

Of the many proteins tested for their capacity to induce collagen accumulation by animal cells, microperoxidase is useful in applications where the size of the active agent is a limiting factor for effective delivery to the desired site of action, for example in epicutaneous or transdermal applications. Microperoxidase is one of the smallest proteins with peroxidase and ECM-inducing activity identified, with a molecular weight of between 1506.48 for MP-8 to 1861.92 for MP-11. The molecular weight is dependent on how many of the peptide residues remain on the fragment of horse heart cytochrome c which has been subject to enzymatic degradation.

Formulations suitable for applying proteins with peroxidase activity to skin.

All units for ingredients of the compositions are measured in “parts”. The formulations are prepared in a manner well known to those skilled in the art, in particular by mixing the constituents if appropriate at elevated temperatures although care should be taken not to elevate the temperature of solutions containing proteins with peroxidase activity towards their denaturation temperature. The oily and aqueous phases are prepared separately and mixed or emulsified as necessary.

The formulations can be prepared such that they vary in strength. The amount of proteins with peroxidase activity used will generally range from between 0.1% and about 75% weight/volume dependent on the type of the formulation, the purity, activity and type of protein having peroxidase activity selected, the amount sufficient to facilitate delivery of proteins with peroxidase activity, the frequency of application, the intended use and the desired result.

(i) Cetomacrogol Cream:

| Agent(s) with peroxidase activity | qs |
|---|---|
| quantum satis (as much as is enough) | |
| Cetomacrogol emulsifying wax | 15 |
| Liquid paraffin (by weight) | 10 |
| Chlorocresol | 0.1 |
| Propylene glycol | 5 |
| Distilled water to | 100 |

(ii) Aqueous Cream APF:

| Agent(s) with peroxidase activity | qs |
|---|---|
| Emulsifying ointment | 30 |
| Glycerol | 5 |
| Phenoxyethanol | 1 |
| Distilled water to | 100 |

(iii) Buffered Cream BPC 73:

| Agent(s) with peroxidase activity | qs |
|---|---|
| Citric acid | 5 |
| Sodium phosphate | 25 |
| Chlorocresol | 1 |
| Emulsifying ointment | 30 |
| Distilled water to | 100 |

(iv) Emulsifying Ointment APF:

| Agent(s) with peroxidase activity | qs |
|---|---|
| Emulsifying wax | 30 |
| White soft paraffin | 50 |
| Liquid paraffin (by weight) | 20 |

(v) Peptide Ointment (as in Neomycin and Bacitracin Ointment BPC 73):

| Agent(s) with peroxidase activity | qs |
|---|---|
| Liquid paraffin | 10 |
| White soft paraffin to | 100 |

(vi) Gel (as used in Lignocaine and Chlorhexidine Gel APF):

| Agent(s) with peroxidase activity | qs |
|---|---|
| Tragacanth | 2.5 |
| Glycerol | 25 |
| Distilled water to | 100 |

(vii) Spray (as used in Adrenaline and Atropine Spray BPC 73):

| Agent(s) with peroxidase activity | qs |
|---|---|
| Sodium metabisulphite | 1 |
| Chlorbutol | 5 |
| Propylene glycol | 50 |
| Distilled water to | 1000 |

(viii) Cetomacrogol Lotion APF:

| Agent(s) with peroxidase activity | qs |
|---|---|
| Cetomacrogol emulsifying wax | 3 |
| Liquid paraffin | 10 |
| Glycerol | 10 |
| Chlorhexidine gluconate solution | 0.1 |
| Distilled water to | 100 |

(ix) W/O Cream:

| Agent(s) with peroxidase activity | qs |
|---|---|
| Glycerol sorbitan fatty acid ester (Arlacel 481) | 6 |
| Microcrystalline wax (Lumacera M) | 1 |
| Neutral oil | 3 |
| Paraffin oil | 19 |
| Magnesium stearate | 1 |
| Propylene glycol | 3.7 |
| Magnesium sulphate | 0.7 |
| Distilled water to | 100 |

(x) W/O Emulsion:

| Agent(s) with peroxidase activity | qs |
|---|---|
| Polyoxyethylene glycerol sorbitan fatty acid | 3.6 |
| Ester (Arlacel 988) | |
| Polyoxyethylene fatty acid ester (Arlacel 989) | 1.4 |
| Cetearyl alcohol (Lanette O) | 2 |
| Mineral oil, DAB 9 | 25 |
| Paraben mixture | as desired |
| Magnesium sulphate | 0.7 |
| Distilled water to | 100 |

(xi) W/O Lotion:

| Agent(s) with peroxidase activity | qs |
|---|---|
| Glycerol sorbitan fatty acid ester (Arlacel 481) | 1.3 |
| Polyoxyethylene fatty acid ester (Arlacel 989) | 3.7 |
| Neutral oil (Myglyol) | 6 |
| Paraffin oil, DAB 9 | 14 |
| Propylene glycol | 3.8 |
| Magnesium sulphate | 0.7 |
| Distilled water to | 100 |

(xii) O/W Emulsion:

| Agent(s) with peroxidase activity | qs |
|---|---|
| PEG 100 stearate (Arlacel 165) | 5 |
| Cetearyl alcohol (Lanette O) | 3 |
| Mineral oil, DAB 9 | 25 |
| Paraben mixture | as required |
| Distilled water to | 100 |

(xiii) O/W Emulsion:

| Agent(s) with peroxidase activity | qs |
|---|---|
| Polysorbate 60 (Tween 60) | 3 |
| Sorbitan stearate (Arlacel 60) | 2 |
| Cetearyl alcohol (Lanette O) | 3 |
| Mineral oil, DAB 9 | 25 |
| Paraben mixture | as required |
| Distilled water to | 100 |

(xiv) Cationic Emulsion:

| Agent(s) with peroxidase activity | qs |
|---|---|
| Distearyldimethylammonium chloride | 5 |
| Vaseline, DAB 9 | 5 |
| Isopropyl palmitate | 2 |
| Cetyl alcohol | 1 |
| Silicone oil | 0.1 |
| Propylparaben | 0.1 |
| Methylparaben | 0.1 |
| Glycerol | 4 |
| Distilled water to | 100 |

(xv) Ionic Emulsion:

| Agent(s) with peroxidase activity | qs |
|---|---|
| Sodium cetearyl sulphate (Emulgade F) | 6 |
| Mineral oil, DAB 9 | 25 |
| Paraben mixture | as required |
| Distilled water to | 100 |

(xvi) Ionic O/W Emulsion:

| Agent(s) with peroxidase activity | qs |
|---|---|
| Stearic acid | 5 |
| Cetearyl alcohol (Lanette O) | 3 |
| Mineral oil, DAB 9 | 25 |
| Paraben mixture | as required |
| Triethanolamine | 1 |
| Distilled water to | 100 |

(xvii) Aqueous Formulation (Face Lotion):

| Agent(s) with peroxidase activity | qs |
|---|---|
| PEG 40-hydrogenated castor oil | 0.811 |
| Dipropylene glycol | 2.534 |
| PEG 8 | 1.521 |
| Na₃EDTA | 0.253 |
| Polymer JR 125 | 0.025 |
| Distilled water to | 100 |

(xviii) Aqueous Composition:

| Agent(s) with peroxidase activity | qs |
|---|---|
| Poly-fatty acid ester (Cetiol HE) | 16 |
| PPG 3 myristyl ether (Witconol APM) | 1 |
| Propylene glycol | 3 |
| Glycerol | 40 |
| Distilled water to | 100 |

(xix) Formulation of High Water Content (Very Soft):

| Agent(s) with peroxidase activity | qs |
|---|---|
| Ceteareth (Cremophor A 25) | 0.1 |
| Cetearyl alcohol (Lanette O) | 0.4 |
| Vaseline, DAB 9 | 12.5 |
| Mineral oil, DAB 9 | 11 |
| Ceteareth-6-stearyl alcohol (Cremophore A6) | 6 |
| Distilled water to | 100 |

(xx) Formulation of High Water Content (Soft):

| Agent(s) with peroxidase activity | qs |
|---|---|
| Ceteareth-25 (Cremophor A 25) | 1.5 |
| Cetearyl alcohol (Lanette O) | 8.5 |
| Distilled water to | 100 |

(xxi) Formulation of High Water Content (Soft):

| Agent(s) with peroxidase activity | qs |
|---|---|
| Ceteareth-25 (Cremophor A 25) | 2 |
| Cetearyl alcohol (Lanette O) | 8 |
| Vaseline, DAB 9 | 10 |
| Mineral oil, DAB 9 | 10 |
| Distilled water to | 100 |

(xxii) Formulation of High Water Content (Medium-Firm):

| Agent(s) with peroxidase activity | qs |
|---|---|
| Ceteareth-25 (Cremophor A 25) | 3 |
| Cetearyl alcohol (Lanette O) | 17 |
| Distilled water to | 100 |

(xxiii) Thinly Liquid Lotion:

| Agent(s) with peroxidase activity | qs |
|---|---|
| Ceteareth-25 (Cremophor A 25) | 1 |
| Ceteareth-6-stearyl alcohol | 1 |
| Glycerol mono/distearate (Tegin normal) | 2 |
| Cetyl alcohol | 1 |
| Isopropyl myristate | 1.45 |
| Glycerol | 1 |
| Polyvinylpyrrolidone | 0.5 |
| Distilled water to | 100 |

(xxiv) Viscous Lotion:

| Agent(s) with peroxidase activity | qs |
|---|---|
| Ceteareth-25 (Cremophor A 25) | 2 |
| Cetearyl alcohol (Lanette O) | 3 |
| Mineral oil, DAB 9 | 5 |
| Propylene glycol | 3 |
| Polyvinylpyrrolidone | 0.5 |
| Distilled water to | 100 |

Finally, it will be appreciated that various modifications and variations of the methods and compositions of the invention described herein will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are apparent to those skilled in the art are intended to be within the scope of the present invention.

1.-88. (canceled)

89. A method of promoting production of one or more components of extracellular matrix by one or more fibroblast cells in a biological system and/or promoting migration of one or more fibroblast cells in a biological system, the method including exposing the one or more fibroblast cells in the biological system to an effective amount of an agent with peroxidase activity.

90. The method of claim 89, wherein the one or more components of extracellular matrix are selected from one or more of the group consisting of collagen I, collagen III, collagen IV, collagen VI, fibronectin, elastin, laminin, proteoglycan, hyaluronic acid, and de novo extracellular matrix, or a functional component thereof.

91. The method of claim 89, wherein the agent with peroxidase activity comprises a protein with peroxidase activity.

92. The method of claim 91 wherein the protein with peroxidase activity comprises an enzyme classified under one or more of the following EC numbers: EC 1.11.1.1; EC 1.11.1.2; EC 1.11.1.3; 1.13.11.11; EC 1.11.1.5; EC 1.11.1.7; EC 1.11.1.8; EC 1.11.1.9; EC 1.11.1.10; EC 1.11.1.12; EC 1.11.1.13; EC 1.11.1.14; EC 1.11.1.15; EC 1.11.1.16; or an active fragment or variant of any of the aforementioned.

93. The method of claim 89, wherein the biological system comprises a human or animal subject.

94. The method of claim 93, wherein the exposure of the one or more fibroblast cells to the agent comprises delivery of the agent to a tissue of the subject.

95. The method of claim 93, wherein the delivery of the agent comprises implantation into a tissue and/or implantation in a region near a tissue in the subject.

96. The method of claim 93, wherein the delivery of the agent comprises topical administration to a tissue and/or topical administration to a region near a tissue in the subject.

97. The method of claim 94, wherein the agent is delivered in conjunction with, or associated with, a substrate.

98. The method of claim 97, wherein the substrate is a liquid, a gel, a semi-solid substrate, or a solid substrate.

99. The method of claim 97, wherein the substrate is biocompatible and/or biodegradable.

100. The method of claim 94, wherein the agent is delivered in conjunction with one or more of a steroidal anti- inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent or a chemotactic agent.

101. The method of claim 93, wherein the method promotes production of one or more components of extracellular matrix in a tissue of the subject and/or promotes migration of one or more fibroblast cells into a tissue of the subject.

102. The method of claim 93, wherein the method promotes one or more of a fibrogenic response, tissue generation, tissue regeneration, tissue repair, healing of a wound, rectification of a dermal deficit, or augmentation of a tissue in the subject.

103. Use of an agent with peroxidase activity in the preparation of a medicament for promoting one or more of a fibrogenic response, tissue generation, tissue regeneration, tissue repair, healing of a wound, rectification of a dermal deficit, or augmentation of a tissue in a subject.

104. A composition comprising an agent with peroxidase activity.

105. The composition of claim 104 wherein the agent comprises a protein with peroxidase activity.

106. The composition of claim 105 wherein the protein with peroxidase activity comprises an enzyme classified under one or more of the following EC numbers: EC 1.11.1.1; EC 1.11.1.2; EC 1.11.1.3; 1.13.11.11; EC 1.11.1.5; EC 1.11.1.7; EC 1.11.1.8; EC 1.11.1.9; EC 1.11.1.10; EC 1.11.1.12; EC 1.11.1.13; EC 1.11.1.14; EC 1.11.1.15; EC 1.11.1.16; or an active fragment or variant of any of the aforementioned.

107. The composition of claim 104 wherein the composition further comprises one or more of: a liquid substrate, a gel substrate, a semi-solid substrate, or a solid substrate.

108. The composition of claim 107 wherein the substrate is biocompatible and/or biodegradable.

109. The composition of claim 104, wherein the composition further comprises one or more of a steroidal anti-inflammatory drug, a calcineurin inhibitor, an anti-histamine, an anti-microbial agent, an antibiotic, a growth factor, a growth promoting agent, an angiogenic promoter, a protease inhibitor, an anti-oxidant, an anaesthetic agent, an analgesic agent or a chemotactic agent.

110. The composition of claim 104 when used according to the method of claim 1.
